(19) United States
(12) Patent Application Publication
(10) Pub. No.: US 2004/0071665 A1
(43) Pub. Date: Apr. 15, 2004

(54) Method for therapeutically treating a clinically recognized form of cardiopathology in a living mammal

(76) Inventors: Yong-Fu Xiao, Wayland, MA (US); James P. Morgan, Newton Centre, MA (US)

Correspondence Address: David Prashker, Esq., DAVID PRASHKER, P.C., P.O. Box 5387, Magnolia, MA, 01930, US

(21) Appl. No.: 10/438,574

(22) Filed: May 15, 2003

Related U.S. Application Data
(63) Continuation of application No. PCT/US02/07555, filed on Mar. 14, 2002.
(63) Continuation-in-part of application No. 09/684,679, filed on Oct. 7, 2000, now Pat. No. 6,607,720.
(63) Continuation-in-part of application No. 09/655,124, filed on Sep. 5, 2000, now Pat. No. 6,534,052.

(51) Int. Cl. A61K 48/00

(52) U.S. Cl. 424/93.7

(57) ABSTRACT

The present invention provides therapeutic methods which employ one or more identifiable types of mammalian stem cells, and/or their progenitor progeny cells, and/or their lineage-committed descendant cells, and/or their partially-differentiated offspring cells—with or without completely differentiated cells—to treat living mammalian subjects afflicted with a clinically recognized form of cardiopathology. The identifiable cell types include embryonic stem cells and their offspring cells; as well as the presently identified types of adult stem cells and their various offspring cells; and also include recently identified alternative cell types which have functional stem cell properties. Among the clinical forms of cardiopathology which can be efficaciously treated using the present therapeutic methods are myocardial infarction, myocarditis, heart failure, and cardiac dysrhythmia.

### PROVISIONAL PATENT APPLICATIONS

The present invention was first filed with the U.S. Patent and Trademark Office on Mar. 15, 2001 as U.S. Provisional Patent Application Nos. 60/276,148; 60/276,147; 60/276,247; 60/276,246; 60/276,245; 60/276,244; 60/276,243; and 60/276,175 respectively.

### CROSS-REFERENCE

The present application is a Continuation-In-Part of U.S. patent application Ser. No. 09/655,124 filed Sep. 5, 2000, now pending; and of Ser. No. 09/684,679 filed Oct. 7, 2000, now pending.

### BACKGROUND OF THE INVENTION

Cardiac dysfunction is a life-threatening event that may cause sudden death and heart failure. Despite considerable advances in the diagnosis and treatment, cardiac dysfunction and cardiopathology remains a worldwide problem that is increasing in incidence, prevalence, and overall mortality. Finding new effective therapeutic approaches to improve cardiac dysfunction therefore remains a major challenge.

Cell transplantation has emerged as a potentially new approach of repairing damaged myocardium in recent years. Transplanted cardiomyocytes have been shown to survive, proliferate, and connect with the host myocardium in murine models. Li and his coworkers demonstrated that transplanted fetal cardiomyocytes could form new cardiac tissue within the myocardial scar induced by cryoinjury and improve heart function [Li et al., Cir. Res. 78: 283-288 91996); and Ann. Thorac. Surg. 62:654-661 (1996)]; but the transplanted allogenic cells survived for only a short period in the recipient heart due to immunorejection [Li et al., Circulation 96 (Suppl. II): II179-II187 (1997)]. Bishop et al. reported that the embryonic myocardium of rats can be implanted and cultured in oculo [Bishop et al., Circ. Res. 66: 84-102 (1990)] and demonstrated that the engrafted embryonic cardiomyocytes proliferated and differentiated. In a recent review, Heschler et al. pointed out that totipotent embryonic stem (ES) cells cultivated within embryonic bodies reproduce highly specialized phenotypes of the cardiac tissue [Heschler et al., Cardiovas. Res. 36: 149-162 (1997)]. Most of the biological and pharmacological properties of cardiac-specific ion currents were expressed in cardiomyocytes developed in vitro from pluripotent ES cells, which were similar to those previously described in adult cardiomyocytes or neonatal mammalian heart cells. However, the significance of ES cell transplantation in heart disease remains to be examined.

Several other studies have demonstrated the feasibility of engrafting exogenously supplied cells into host myocardium, including fetal cardiomyocytes [Soonpaa et al., Science 264: 98-101 (1994)] derived from artial tumor (AT1), satellite cells [Chiu et al., Ann. Thorac. Surg. 60: 12-18 (1995)], or bone marrow cells [Tomita et al., Circulation 100 (Supp. II): II247-II256 (1999)]. These engrafted cells have been histologically identified in normal myocardium up to 4 months after transplantation. Gap junctions have also been found between the engrafted fetal cardiomyocytes and the host myocardium [Soonpaa et al., Science 264: 98-101 (1994)], thereby raising the possibility of electrical-contraction coupling between transplanted cells and the host tissue. Recently, myocyte transplantation has been extended into ischemically damaged myocardium with coronary artery occlusion in rats [Scorsin et al., Circulation 94 (Suppl II): II337-II340 (1996)]; or with cryoinjury in rats [Li et al., Ann. Thorac. Surg. 62: 654-661 (1996) and Cir. Res. 78: 283-288 (1996)]; and in dogs [Chiu et al., Ann. Thorac. Surg. 60:12-18 (1995)].

Nevertheless, despite all these research efforts and reported investigations, very little progress has been made to date in methods and cellular materials which might directly and markedly improve cardiac function in the living host after the occurrence of clinical cardiopathology; or might serve as a cell transplantation therapeutic technique for effecting at least a partial repair of the myocardium; or might offer a potential long-term improvement of the damaged heart tissue in the afflicted host subject. Were such an effective methodology to be generated and empirically demonstrated, such a development would be regarded as a major advance and unforeseen event by physicians and surgeons working in the field of cardiology.

### SUMMARY OF THE INVENTION

The present invention has multiple aspects and in-vivo applications. A first aspect provides a therapeutic method for decreasing the severity of cardiac dysfunction in a living mammalian subject afflicted with a clinically recognized form of cardiopathology, said method comprising the steps of:

introducing a prepared inoculum of viable mammalian cells to at least a portion of the heart in the afflicted living subject, said prepared inoculum being a mixture of mammalian cells comprising not less than two different types of cells in combination, said two different types being selected from the group consisting of at least one identifiable type of stem cells, at least one identifiable type of progenitor cells, at least one identifiable type of lineage-committed cells, at least one identifiable type of partially-differentiated cells, and at least one identifiable type of completely differentiated cells; and

allowing said introduced inoculum of cells to develop in-situ as integrated cells within and about the heart, whereby the severity of cardiac dysfunction becomes decreased in the afflicted living subject.

A second aspect of the invention provides a therapeutic method for decreasing the severity of cardiac dysfunction in a living mammalian subject afflicted with a clinically recognized form of cardiopathology, said method comprising the steps of:

introducing a prepared inoculum of viable mammalian cells to at least a portion of the heart in the afflicted living subject, said prepared inoculum being a mixture of mammalian cells comprising not less than two different types of cells in combination, said two different types being selected from the group consisting of one or more identifiable types of stem cells, one or more identifiable types of progeny cells, one or more identifiable types of lineage-committed cells, and one or more identifiable types of partially-differentiated cells; and

allowing said introduced inoculum of cells to develop in-situ as integrated cells within and about the heart, whereby the severity of cardiac dysfunction becomes decreased in the afflicted living subject.

A third aspect of the present method provides a therapeutic method for decreasing the severity of cardiac dysfunction in a living mammalian afflicted with a clinically recognized form of cardiopathology, said method comprising the steps of:

introducing a prepared inoculum of viable mammalian cells to at least a portion of the heart in the afflicted living subject, said prepared inoculum being a mixture of mammalian cells comprising not less than three different types of cells in combination, said three different types being selected from the group of one or more identifiable types of stem cells, one or more identifiable types of progeny cells, one or more identifiable types of lineage-committed cells, one or more identifiable types of partially-differentiated cells, and one or more identifiable types of completely differentiated cells; and

allowing said introduced inoculum of viable mammalian cells to develop in-situ as integrated cells within and about the heart, whereby the severity of cardiac dysfunction becomes decreased in the afflicted living subject.

### BRIEF DESCRIPTION OF THE FIGURES

The present therapeutic methodology can be more easily understood and is better appreciated when taken in conjunction with the accompanying drawing, in which:

FIGS. 1A-1C respectively are illustrations showing the spontaneous action potentials observed using the zero-current clamp method in ESCs-derived cardiomyocytes;

FIGS. 2A-2C respectively are illustrations showing original trace recordings of hemodynamic measurements in myocardial infarcted mice;

FIG. 3 is a graph showing the increase in developed tension to isoproterenol stimulation in papillary muscles of mice under test;

FIGS. 4A-4C respectively are images showing hematoxylin and cosin stained mouse heart tissues;

FIGS. 5A-5C respectively are images showing marker GFP expression in cultured ESCs and in myocardium after cell transplantation;

FIGS. 6A-6F respectively are images of immunostaining for cTn-I and α-MHC in mouse hearts;

FIG. 7 is an image showing immunofluorescence labeling of engrafted cells and connexin 43 in injured myocardium;

FIGS. 8A-8D respectively are images showing the expression of VEGF in cultured ESCs with and without transfection of phVEGF₁₆₅;

FIGS. 9A and 9B respectively are graphs comparing left ventricular function in myocardial infarcted mice;

FIGS. 10A and 10B respectively are illustrations showing the effects of ESC transplantation on capillary density in infarcted myocardium;

FIGS. 11A-11C respectively are images showing the positive staining for blood vessel endothelial cells by anti-von Willebrand Factor antibody in mouse myocardial sections;

FIG. 12 is a graph showing the various Kaplan-Meier survival curves for the different groups of rats under test;

FIGS. 13A-13C respectively are graphs showing that ES cell transplantation significantly improved left ventricular function in post-infarcted rats;

FIGS. 14A-14C respectively are images showing representative echocardiographic recordings for the different groups of rats under test;

FIGS. 15A-15C respectively are images showing GFP positive spots and single cells from infarcted myocardium after cell transplantation;

FIGS. 16A-16H respectively are images showing engrafted ES cells in post-infarcted rat myocardium identified by hematoxylin-eosin staining at 32 weeks after transplantation;

FIG. 17 is a graph showing the significant increase of capillaries in damaged myocardium in myocardial infarcted rats after ES cell transplantation;

FIG. 18 is a graph showing the survival of the different groups of mice under test;

FIGS. 19A-19I respectively are images showing hematoxylin and eosin stained myocardial sections from ES cell-treated myocarditis mice;

FIGS. 20A and 20B respectively are graphs showing the histological grading of infected mice hearts;

FIGS. 21A-21H respectively are images of myocardial sections from viral myocarditic myocardium treated with embryonic stem cells and their progenitor progeny cells at day 14 after infection;

FIGS. 22A-22D respectively are graphs showing hemodynamic measurements in post-infarcted porcine hearts under various test conditions;

FIGS. 23A-23F respectively are images showing the morphology of porcine myocardium as stained sections taken from the animals under test;

FIGS. 24A-24F respectively are images showing the immunostaining for α-cardiac heavy chain and cardiac troponin I in the myocardium of the animals under test;

FIGS. 25A-25C respectively are images showing the immunostaining for GFP and cTnI in infarcted myocardium having transplanted hMSCs and hFCs; and

FIGS. 26A and 26B are graphs showing the observed blood flow measurements with the neutron microsphere technique in post-infarcted porcine hearts at resting condition and with pacing stress.

### DETAILED DESCRIPTION OF THE INVENTION

The present invention is a therapeutic method which employs one or more mammalian category types of pluripotent stem cells and their multipotent progenitor progeny cells—with or without inclusion of their lineage-committed, but undifferentiated descendant offspring cells—to treat living mammalian subjects afflicted with a clinically recognized form of cardiopathology. The cell category types include embryonic stem cells and their offspring cells; as well as adult stem cells and their various offspring cells. Among the clinically manifested forms of cardiopathology able to be therapeutically treated are myocardial infarctions, myocarditis, heart failure, and cardiac dysrhythmia. A detailed presentation and disclosure of the clinically recognized cardiopathology forms, the individual cell types; and the therapeutic methodology is given below. This descriptive written disclosure is then augmented and supported by a range and variety of experiments and empirical data which are both representative and indicative of the scope for the treatment method.

### I. A Clinically Recognized Type of Cardiopathology

There are many types of cardiac dysfunction, pathologies and heart disease, which can be clinically and diagnostically distinguished. The signs of cardiac dysfunction and heart disease appear as various manifestations and symptoms, each of which represents a distinct form of cardiopathology. The most common symptoms in man are dyspnea, chest pain, palpitations, presyncope or syncope, and fatigue. None are specific; and interpretation depends on assessing the entire clinical picture; and, in many instances, diagnostic testing.

For these reasons, the therapeutic methods comprising the present invention are particularly concerned with not less than four different forms of mammalian heart problems, each of which is a recognized type of cardiopathology. Each medical problem is described in detail below.

### A. The Medical Problem of Acute Myocardial Infarction

### Definition

The sudden occlusion of a coronary artery leading to myocardial death.

### Etiology

The immediate mechanism is rupture of an atheromatous plaque causing thrombosis and occlusion of coronary arteries and myocardial death. Factors that convert a stable plaque into an unstable plaque may include shear stresses, inflammation, and autoimmunity.

### Prognosis

Acute myocardial infarction (AMI) may lead to a host of mechanical and electrical complications, including death, ventricular dysfunction, congestive heart failure, cardiogenic shock, fatal and non-fatal arrhythmia, valvular dysfunction, or myocardial rupture. Acutely and sub-acutely, myocardial dysfunction may result from the loss of functioning myocytes due to:

1) Necrosis (irreversible damage; myocytes in Adult mammals do not demonstrate significant ability to enter cell cycle and divide);

2) Myocardial “stunning” (a temporary state of unknown mechanism that may be protective if adequate heart function remains in which decreased contractility is present in peri-infarction tissue.

Typically, function will return to normal in days, weeks or months after the infarct). Chronically, cardiac function may also be depressed by adverse remodeling of the heart that occurs due to increased stresses (mostly hemodynamic) on the heart combined with an ineffective or pathologic compensatory response (i.e., excessive fibrosis or inadequate hypertrophy or pathologic hypertrophy), which includes compensatory hypertrophy. Adverse remodeling produces a cardiac configuration that is sub-optimal for pump function (normal prolate ellipsoidal configuration becomes more spheroidal, with thinned and possibly anerysmal ventricular [muscle] walls).

### Aims of the Therapeutic Method

The primary therapeutic aim is to prevent myocardial necrosis and/or restore physiologic cardiac function to normal as assessed by performance on diagnostic stress testing. Additional aims are: to relieve pain; to restore blood supply to the heart muscle; to reduce incidence of complications (see above); to prevent recurrent ischemia and infarction; to prevent or reverse adverse remodeling and cardiac dysfunction; and to decrease mortality.

### Efficacious Outcomes

The rates of major cardiovascular events (including death, recurrent AMI, refractory ischemia, and stroke) are markedly decreased. A maximal preservation of cardiac function (without increasing work of heart or reversing protective effect of stunning) and prevention of adverse remodeling would also occur.

### Advantages and Benefits

The rates of major bleeding and intracranial hemorrhage (typically from revascularization procedures) are greatly reduced. There is a minimum of pharmacologically active drugs and devices utilized.

### B. The Medical Problem of Myocarditis

### Definition

Myocarditis is an inflammatory disease of the heart. It can be acute, subacute, or chronic; and there may be either focal or diffuse involvement of the myocardium. Both systemic and cardiac symptoms may be seen. In the early stages of viral myocarditis, for example, the patient may have fever, myalgias, and muscle tenderness noted. The muscle symptoms are attributable to myositis induced by a myotrophic virus. In symptomatic patients, the cardiac presentation may be one of an acute cardiomyopathy.

### Etiology

Myocarditis may be caused by infectious organisms such as viruses, bacterias, fungi, protozoa, and helminths; or by a toxin such as cocaine. Myocarditis can also be associated with systemic illness including granulomatous, collagen-vascular, and autoimmune diseases.

Viral infection is the most common cause of myocarditis. The most frequently implicated viruses are Coxsackievirus B, echovirus, influenza virus, Epstein-Barr virus, and the viruses of childhood exanthematous diseases. However, all pathogenic viruses may replicate in the heart and induce myocarditis.

### Pathology

Myocarditis may be focal or diffuse, involving any or all cardiac chambers. Severe diffuse myocarditis can result in dilatation of all cardiac chambers; and there may be mural thrombus formation in any chamber.

Histological examination reveals cellular infiltrates, which are usually mononuclear, but may be neutrophilic or occasionally eosinophilic. The infiltrates are of varying severity; and are often associated with myocyte necrosis and disorganization of the myocardial cytoskeleton. With subacute and chronic myocarditis, interstitial fibrosis may replace fiber loss, and myofiber hypertrophy may be seen.

### Dallas Criteria

Variability in the interpretation of endomyocardial biopsy led a panel of cardiac pathologists to develop a working standard, now called the “Dallas criteria”. These criteria are now used by most investigators to define the disease as acute or borderline myocarditis.

Active myocarditis is defined as “an inflammatory infiltrate of the myocardium with necrosis and/or degeneration of adjacent myocytes not typical of the ischemic damage associated with coronary heart disease”.

Borderline myocarditis is the term used when the inflammatory infiltrate is too sparse, or myocyte injury is not demonstrated. Repeat biopsy is recommended when borderline myocarditis is seen; in one reported study, active myocarditis was demonstrated on repeat biopsy in 4 of 6 patients.

Also, studies evaluating the diagnostic efficacy of endomyocardial biopsy, using autopsy as the standard, have estimated that this technique has only a 63% specificity and a 79% sensitivity for the diagnosis of myocarditis. This may be due in part to the focal and transient nature of histologic abnormalities diagnostic of myocarditis which may largely resolve within as little as 4 days after the initial biopsy.

### Pathogenesis

The myocarditis process in the majority of cases is thought to be initiated by an infectious agent (usually viral). Both direct viral-induced myocyte damage and post-viral immune inflammatory reactions contribute to myocyte damage and necrosis. Inflammatory lesions and the necrotic process may persist for months, although viruses only replicate in the heart for at most 2 or 3 weeks after infection.

A variety of findings indicates a pathogenic role for immune or autoimmune processes. These include infiltration with predominately T-lymphocytes; the presence of activated macrophages, B-cells, cytokines, and adhesion molecules; and the expression of major histocompatibility complex antigens in the myocardium.

The immunopathogenesis of myocarditis initiated by viral infection was first demonstrated and characterized in experimental models, and then confirmed in humans. Circulating autoantibodies have been demonstrated in patients with myocarditis and may persist for a prolonged period. The antibodies are directed against mitochondria and contractile proteins and the beta-adrenergic receptors. As one example, one study found that 25% of patients with a dilated cardiomyopathy and 10% of patients with an ischemic cardiomyopathy had anti-beta-l-adrenoceptor antibodies. These antibodies were not found in-patients with a cardiomyopathy secondary to valvular or hypertensive heart disease.

Evidence from experimental models of myocarditis has also incriminated cytokines (such as interleukin-1 and tumor necrosis factor) oxygen-free radicals, and microvascular changes as contributory pathogenic factors. These studies have also identified factors that can enhance the susceptibility to and severity of myocarditis. These factors include forced exercise; pregnancy; nutritional deficiencies; and the ingestion of ethanol, steroids, and non-steroidal anti-inflammatory drugs.

### Aims of the Therapeutic Treatment

The primary therapeutic aim is to prevent myocardial necrosis and/or restore physiologic cardiac function to normal as assessed by performance on diagnostic stress testing. Additional aims of the methodology are to prevent or reverse heart failure, arrhythmia and other morbidity associated with myocarditis.

### C. The Medical Problem of Heart Failure

### Definition

Heart failure occurs when abnormality of cardiac function causes a failure of the heart to pump blood at a rate sufficient for metabolic requirements, or maintains cardiac output only with a raised filling pressure. It is characterized clinically by breathlessness, effort intolerance, fluid retention, and poor survival.

### Etiology

Coronary artery disease is the most common cause. Other common causes include hypertension, myocardistis and valve disease; many cases are idiopathic.

### Types

Heart failure can be caused by systolic or diastolic dysfunction and is associated with neurohormonal changes. Left ventricular systolic dysfunction is defined as left ventricular ejection fraction below 40%. It can be symptomatic or asymptomatic.

Defining and diagnosing diastolic heart failure can be difficult. The criteria include evidence of:

1) Heart failure;

2) Normal or mildly abnormal left ventricular systolic function

3) Abnormal left ventricular relaxation, filling, diastolic distensibility, or diastolic stiffness.

A person meeting all three criteria would have definite diastolic heart failure; a person meeting the first and second criteria only would indicate a possible diastolic heart failure.

Diastolic and systolic dysfunction are usually present together in most patients to varying degrees. It is envisioned that the transplantation of stem cells and progenitor cells may be most useful in correcting systolic dysfunction, but the introduction of such cells in-situ may also benefit diastolic dysfunction (by improving distensibility).

### Aims of the Therapeutic Method

To relieve symptoms; to improve quality of life; to reduce morbidity and mortality, with minimum adverse effects.

### Efficacious Outcomes

Functional capacity can be assessed by: The NY Heart Association functional classification; or more objectively evaluated by using the standardized exercise-testing program; or the 6 minute walk test [Bittner et al., JAMA 270: 1702-1707 (1993)] or determined by Qualify of life as assessed with questionnaires [Rogers et al., J Am Coll Cardiol 23: 393-400 (1994)]; or mortality rates.

### D. The Medical Problem of Cardiac Dysrhythmias

### Definition

Cardiac dysrhythmias (inaccurately termed arrhythmias in common parlance) are any abnormality in the rate, regularity, or sequence of cardiac activation.

### Etiology

Disorders of heart rhythm result from alterations of impulse formation, impulse conduction, or both. Electrical impulse formation in the heart arises from the intrinsic automaticity of specialized cardiac cells. Automaticity refers to a cell's ability to depolarize itself to a threshold voltage in a rhythmic fashion, such that spontaneous action potentials are generated. Although atrial and ventricular myocytes do not have this property under normal conditions, the cells of the specialized conducting system do possess natural automaticity, and are therefore termed pacemaker cells.

The specialized conducting system includes the SA node, AV node, and the ventricular conducting system. The latter is composed of the Bundle of His, the bundle branches, and the Purkinje fibers. In pathologic situations, myocardial cells outside the conducting system may also acquire the property of automaticity.

The primary causes of arrhythmias are:

1) Altered impulse formation;

2) Altered impulse conduction.

Abnormalities of impulse formation and/or conduction may result in a cardiac rhythm that is too fast, slow or irregular to allow the heart to meet the metabolic demands of the body. Clinically, these abnormalities may manifest as light-headed episodes, palpitations, pre-syncope or syncope. When dysrhythmias occur, the perfusion of the heart and other vital organs may be impaired resulting in ischemia and infarction or other forms of injury.

Arrhythmias are diagnosed with an EKG or 24 hour Holter monitor (continuous recording of cardiac cycles) or intermittent recording performed by the patient with an ambulatory monitor (“King of Hearts”) when symptoms occur. The normal heart rate range is 60-100 beats/min. When the heart rate (normal or arrhythmic) is greater than 100 bpm, the patient is said to have a “tachycardia” or “tachyarrhythmia”; when less than 60 bpm, a “bradycardia” or “bradyarrhythmia”.

### Conventionally Known Treatments

Conventional treatment may be pharmacological or mechanical. Often, slow rhythms occur as primary component of arrhythmia or as complication of therapy for fast rhythms. When patients become symptomatic from a slow heart rate, an electrical pacemaker is often implanted to maintain the heart rate in the normal range. These are expensive devices that require frequent checking for mechanical problems, battery charge and effective stimulation of the heart. Use requires placement of a wire in the heart, which can become infected. Although current generation devices are compact, they do produce cosmetic disturbances at the site if implantation under the skin, and may elicit an inflammatory response.

### Aims of the Therapeutic Treatment Method

The primary therapeutic aim is to restore regular cardiac rate and rhythm, thereby preserving or restoring the normal physiological function of the heart as assessed by acute and long-term monitoring of the electrocardiogram and/or advanced electrophysical study. The implantation of pluripotent stem cells and their living progeny that can be stimulated in-situ to differentiate into pacemaker cells offers an alternative to electrical pacemakers that has a lower incidence of adverse effects and which requires less follow up. In addition, the implantation of such stem cells and their living progeny may also restore normal impulse conduction (rate and rhythm) to the heart. Appropriate cells would be preferably injected directly into a ventricular or supraventricular site using a common needle syringe or catheter.

### II. The Therapeutic Treatment Method

The methodology for restoring a degree of normalcy for heart activity and function in a mammalian subject suffering from a clinically recognizable form of cardiopathology utilizes one or more identifiable types of viable stem cells, and/or progenitor cells, and/or lineage-committed, and/or partially-differentiated cells collectively, in blended combinations—with and without completely differentiated cells—as a prepared cell inoculum. All of these viable cells have been prepared in advance and maintained in vitro; and the therapeutic method introduces prechosen mixtures of these viable stem cells, progenitor cells and lineage-committed or partially differentiated cells as a commingled admixture of cells—which are then administered by any suitable route and introduced to a preselected, specified anatomic site within the heart or vascular system body of the host recipient as transplanted cells.

After being introduced to the chosen anatomic site in the body of the living recipient as a commingled mixture of cells, these blended stem cells, progenitor cells, and lineage-committed or partially-differentiated cells, not only remain viable in-vivo; but also (i) act therapeutically in-situ to reduce the severity of the pathological condition; (ii) serve to provide healthy, viable cells in-situ as functional cell replacements for previously injured or necrotic cells; and (iii) act therapeutically to increase normal heart and vascular functions for the host recipient as measured by objective and medically recognized clinical test procedures.

### The Different Categories of Stem Cells

At least five different categories or Orders of identifiable stem cells, progenitor cells, lineage-committed cells and partially differentiated cells are now part of the convention paradigm and are deemed to be suitable for individual use. The most preferred single cell category (or Order of cell) is the embryonic stem cell. The embryonic stem cell category together with both its immediate and later generational offspring (the immediate progeny and descendents of embryonic stem cells) constitute one Order. These are deemed most suitable for use as transplanted cells for all types and kinds of cardiac-associated diseases and pathologies generally; and are believed to provide a variety of differentiated descendent cells in-situ which are physiologically functional to alleviate the pathology of the disease state and to improve the clinical status and overall medical condition of the host recipient.

In contrast, four individual and different categories (or Orders) of adult stem cells and offspring cells are presently known. Each of these categories of adult stem cells is separate and distinguishable from all the others; presents unique surface markers and self-identifying cellular attributes; and is conventionally recognized as being able to yield immediate progeny cells and later-generation descendent cells which subsequently become lineage-committed and then differentiated cells having a distinctive morphology and identifiable functional characteristics. These four categories of adult stem cells include: mesenchymal stem cells and their offspring; hematopoietic stem cells and their offspring; neural stem cells and their offspring; and neural crest stem cells and their offspring. Each of these four categories of adult stem cells may used within the therapeutic methodology as a complete substitute for and effective replacement of the most preferred category comprising embryonic stem cells, embryonic progenitor cells, and their lineage-committed or partially-differentiated descendent cells.

In addition, each of these categories of stem cell and their offfspring can be maintained in-vitro as a stable, isolated culture of cells; and propagated in-vitro indefinitely to provide sufficient numbers of viable cells for transplantation and therapeutic use purposes.

### Commingling of Stem Cell Categories as an Alternative Method

The present therapeutic method is a broad-based therapeutic technique—a treatment process which envisions and recognizes that more than a single category of cell and more than one generational stage of a cell type can be commingled and used as a prepared inoculum of cells for clinical treatment purposes. In this alternative mode of practicing the therapeutic treatment method, the preferred embryonic stem cell and embryonic progenitor cells may be intentionally combined and blended with one or more other kinds of cells chosen from among the four categories (or Orders) of adult stem cells. Such blending of stem cell orders is desirably made only immediately before actual administration of the commingled cells [differing as to cell types, immediate origins, and the generational cell stage of development] to the intended host recipient in order to prevent cross-contamination of the individual category types as well as to avoid early cross-influencing effects often caused by the different cell orders and generational stages upon one another after being combined as a prepared admixture of cells.

A large number of stem cell category blends are available; and the numbers of category types employed for any blending can be varied from as few as two (2) types to using all five (5) types in one admixture. For example, the preferred embryonic stem cell and the embryonic progenitor cell can be alternatively and individually combined with any one other partially-differentiated cell taken from any of the other four categories of stem cells to form a range of different cell admixtures as the intended inoculum of cells for the recipient. Thus not less than four different blending varieties comprised of two individual cell types can be prepared: (i) embryonic stem and progenitor cells with mesenchymal derived or partially-differentiated cells; (ii) embryonic stem and progenitor cells with hematopoietic derived or partially-differentiated cells; (iii) embryonic stem and progenitor cells with neural crest derived or partially-differentiated cells; and (iv) embryonic stem and progenitor cells with neural derived or partially-differentiated cells. Each two category cell admixture can be individually prepared in any volumetric quantity or cell ratio on-demand as a blend; and then administered to the recipient host as a purposely prepared admixture of two different orders of cells; which collectively and cumulatively presents in a single prepared inoculum, a stem cell, a progenitor cell—with or without a lineage-committed or partially-differentiated cell—in combination for therapeutic treatment purposes.

In a similar fashion, three stem category mixtures, four stem cell category blends, and even a five stem cell category commingling can be prepared at will; in any desired ratio of stem cell types; and utilized in a preferred mode of administration for therapeutic treatment of a particular patient. In these more elaborate blendings, more than one category of stem cell will be represented; and more than one kind of derived or partially differentiated cell will likely be present within the prepared admixture of cells.

The perceived value and benefit of such blends comprising multiple stem cell admixtures, and/or multiple progenitor cells mixtures, and/or multiple lineage-committed or partially-differentiated cells in a single blended preparation is the greater range and variety of descendent cells that will be produced in-situ; the greater therapeutic effect in-vivo over time; and the far greater diversity of cell functions that will be preformed by the larger variety of cell kinds differentiating in-vivo as replacement cells generated in-situ.

### Genetically Modified Variants of the Native Cells

The recombinant DNA technology conventionally known and available today may be employed at will to generate genetically altered and modified stem cells, and/or progenitor cells, and lineage-committed or partially differentiated cells for each of the five different stem cell categories. Thus, any category type of stem cell and/or progenitor cell and lineage-committed or partially differentiated cell can be transfected with a vector comprising a DNA insert encoding one or more proteins or polypeptides. The DNA sequence coding insert can represent a heterogeneous protein or polypeptide; or may constitute a homogeneous protein or polypeptide which is desirably to be overexpressed by that modified cell.

Typically, the vector comprises additional operatively linked DNA sequences which are required for expression intracellularly of the protein or polypeptide in the transfected cell. “Operatively-linked” means that the substantive DNA sequence is joined to one or more regulatory DNA sequences in a manner that allows intracellular expression of the nucleic acid sequence. Regulatory DNA sequences are art-recognized and may be conventionally chosen at will to produce the desire expression result. Accordingly, the term “regulatory sequence” typically includes promoters, enhancers, and other expression control elements known in this technology. For example, either the native regulatory sequences of the vector or the regulatory sequences of the stem/progenitor/lineage-committted or partially-differentiated cell can be employed.

The genetically modified or altered stem/progenitor/lineage-committted or partially-differentiated cells may be employed therapeutically using the present methodology in the same manner as the native stem cells and progenitor cells and partially-differentiated cells are used. All genetic modification and alterations of such cells should be performed and completed in-vitro and in advance of using those genetically modified cells for therapeutic treatment purposes.

Accordingly, by the very requirements of the present invention it is thus important, if not essential, that the user be at least familiar with the many techniques for manipulating and modifying nucleotides and DNA fragments which have been reported and are today widespread in use and application. Merely exemplifying the many authoritative texts and published articles presently available in the literature regarding genes, DNA nucleotide manipulation and the expression of proteins from manipulated DNA fragments are the following: Gene Probes for Bacteria (Macario and De Marcario, editors) Academic Press Inc., 1990; Genetic Analysis. Principles Scope and Objectives by John R. S. Ficham, Blackwell Science Ltd., 1994; Recombinant DNA Methodology II (Ray Wu, editor), Academic Press, 1995; Molecular Cloning. A Laboratory Manual (Maniatis, Fritsch, and Sambrook, editors), Cold Spring Harbor Laboratory, 1982; PCR (Polymerase Chain Reaction), (Newton and Graham, editors), Bios Scientific Publishers, 1994; and the many references individually cited within each of these publications. All of these published texts are expressly incorporated by reference herein.

In addition, a number of issued U.S. patents and published patent applications have been issued which describe much of the underlying DNA technology and many of the conventional recombinant practices and techniques for preparing genetically altered DNA sequences. Merely exemplifying some of the relevant patent literature for this subject are: U.S. Pat. Nos. 5,486,599; 5,422,243; 5,654,273; 4,356,270; 4,331,901; 4,273,875; 4,304,863; 4,419,450; 4,362,867; 4,403,036; 4,363,877; as well as Publications Nos. W09534316-A1; W09412162-A1; W09305167-A1; W09012033-A1; W09500633; W09412162; and R09012033. All of these patent literature publications are also expressly incorporated by reference herein.

### III. Terminology and Organization of the Stem Cell Paradigm

The therapeutic treatment method comprising the present invention is presented as a somewhat incongruous approach and contrasting position to the conventional opinions and ordinary expectations of practitioners working in this technical field. The present invention will often contravene and stand opposite to conventional views and positions; and provides many striking examples of differences and distinctions of cell qualities and cell attributes not previously recognized or appreciated. For these reasons, among others, it is deemed both valuable and useful to provide the reader first with a stem cell family paradigm and organized system of cellular development which underlies and supports the mode and manner by which the identifiable types of stem cells and their progeny yield initially lineage-committed, then partially-differentiated, and then completely differentiated offspring cells.

### A. The Stem Cell/Progenitor Cell Model and Lineage System of Cellular Development

The substantive value and real significance of the present invention can only be properly recognized and truly appreciated in the context of the model and system of cellular development that these unique stem cells and progenitor cells evidence and embody. Much confusion, misleading views, ambiguity and inconsistency has been reported in the scientific and patent literature; and this difficulty is unfortunately reflected by the overall results reported to date for the different investigative attempts to elucidate and specify the various stages of cellular developmental and for the various cellular outcomes originating with and from the primordial stem cell. For these reasons, the present invention presents and utilizes an organized system of specified developmental stages for the offspring of stem cells; and points out the different cell lineage pathways and many partially-differentiated and fully differentiated cell forms. The model system shows that multiple separate and individual cell lineage pathways exist, each having in common a single stem cell ancestor. Each lineage provides for its own pedigree; and each pathway provides at least one differentiated cell outcome (and typically several different phenotypic forms of differentiation).

### Identifiable Stem Cells

Stem cells and their direct and immediate progeny, the pre-progenitor cells, are the archetype cells. These are two different cell types, both of which are unique in their pluripotent properties; are uncommitted and undifferentiated cells; and are demonstrably able to be implanted in-vivo; and subsequently yield and provide a range of fully differentiated specialized cells which differ in function, in morphology, and in phenotypic cell properties. In this model paradigm, the original primordial source for the entire order of cells is the archetype pluripotent stem cell. By its demonstrable properties and cellular attributes, all true stem cells are: (i) uncommitted and undifferentiated cells; (ii) pluripotent cells having an unlimited proliferation capacity; and (iii) are able to self-renew and self-maintain their existence when replicating by producing two daughter progeny cells, one of which becomes a self-renewed stem cell indistinguishable from its parent, while the other daughter progeny cell becomes a direct and true 2nd generation descendent cell, now designated a “preprogenitor cell”. It is essential to recognize that the daughter self-renewed stem cell is and remains identical to and indistinguishable from its parent stem cell in all respects. This self-renewed daughter stem cell is itself pluripotent; and will itself produce two different types of daughter progeny cells in exactly the same manner as its parent ancestor stem cell. In this self-renewing manner, the total number of stem cells will slowly increase in number over time; but will never exist in true abundant or meaningfully large numbers at any time.

### Identifiable Progenitor Cells

The fate of the other daughter cell, which is now initially designated the “pre-progenitor cell”, becomes markedly different from the ancestor parent stem cell and the one self-renewed daughter cell. In comparison to its own stem cell parent, the pre-progenitor cell daughter has a very large, but limited—i.e., multipotent—proliferation capacity. This direct descendent daughter cell, the true 2nd generation cell type, is itself also a multipotent cell as well as a cell stage which is and remains uncommitted and undifferentiated as such over its lifetime. However, the rate of proliferation for the 2nd generation, daughter pre-progenitor cells is much more rapid than its stem cell parent. Again, stem cells are not found in abundance in any tissue, embryonic or adult. The pre-progenitor cell daughter, however, because it proliferates rapidly and frequently over its very large, but finite, reproductive lifetime gives rise to many progeny cells. These progeny cells [the third generation descendants] produced as offspring by the single pre-progenitor cell daughter also become meaningfully altered in their attributes and capabilities; and thus these 3rd generation daughter cells are now properly termed “primary progenitor cells”. It is this 3rd generation of cells or descendent progeny of the single stem cell ancestor that is believed to be, and often typically is, isolated empirically, sometimes in surprisingly large numbers.

The paramount characteristic and dominant attribute of this 3rd generation cell stage, the “primary progenitor cell”, is not their acknowledged capacity for rapid growth and frequent reproduction in very large (yet finite) numbers; but rather lies is in and is represented by their unique capability to become irreversibly committed to a one preset cell lineage and a single fixed pathway of cell development. Thus, the essential choices and decisions concerning what the final intended outcome of the cellular development process is to be and what cell form and phenotype shall exist as the functional result-occurs by and through the large numbers of cells constituting this 3rd generation cell stage.

Thus, these primary progenitor cells [the 3rd generational stage] rapidly produce in very large numbers over a short time period; and it is via this third generation cell stage of descendancy from a common stem cell ancestor that an irreversible commitment to a fixed lineage sequence and pathway of cellular development occurs. Moreover, it is during this 3rd generational cell stage that the multipotency aspect and capacity (exhibited by the immediate 1st and 2nd ancestor cells) now and forevermore becomes lost. The primary progenitor cell therefore is the cell stage and generation of progeny which becomes influenced by external stimuli and chemical signals in the local environment such that an irreversible cell commitment is made to follow a single specified lineage, a progression which continues until at least one particular form of fully differentiated, phenotypic cell is yielded.

The primary progenitor cell is also able to reproduce itself, both before and after true commitment to an individual cell lineage pathway; but its proliferation capacity—particularly after cell commitment—is believed to be markedly restricted and number limited in comparison to its immediate antecedent predecessor and its single stem cell ancestor.

### Lineage Commitment and the Subsequent Stages of Cell Differentiation

Once cell lineage commitment occurs in the primary progenitor cell, the development of the cell then continues within carefully controlled and prescribed pathway limits. Note however, that cell differentiation as a process and distinct event occurs only after a prior irreversible commitment to a fixed cell lineage pathway has been made. Thus, the choices of phenotypic detail for the cell which occur during the later stages of the differentiation process must always follow the fixed limits set by the earlier event of cell commitment to a specific lineage pathway.

### Identifiable Partially-Differentiated Cells

The partially differentiated cell is one which has undergone cell lineage pathway commitment, but has not yet fully developed its final characteristics and specific phenotypic attributes. It is only at the latter or final stages of the cell differentiation process that a particular morphological appearance, an identifiable functional form and the distinctive phenotypic properties and/or surface markers for that committed cell are decided and brought into existence; and that during the process, first a partially-differentiated cell and then a completely differentiated cell develops and subsequently emerges as the final cell embodiment.

The incremental progression of events, observable as a series of continuing cell stages and developmental cell traits (from early, to middle, to late stages), for cell differentiation has long been recognized and experimentally evaluated, as reported in the conventional scientific literature. One often-seen example of this ever-advancing developmental sequence of events is the particular cell stage often termed a “cardiomyoblast”, or more generally a “myoblast”; and is empirically observed to be those specific cells in a culture medium which show a “beating” or “contracting” appearance and capability. In fact, however, these “beating” cells are actually lineage-committed cells as such and constitute those cells now irreversibly committed to the pathway progression which yeilds either a striated muscle cell, or a smooth muscle cell, or a cardiac muscle cell. For definitional purposes, therefore, a partially-differentiated cell comprises and includes any stage of development and viable existence for any type, stage or category of viable cell which exists after lineage pathway commitment has occurred, but for which the completion of the differentiation process has yet to occur in full phenotypic and idiotypic terms.

### B. The Presently Evolving Concept of an Identiable Type of Stem Cell

Many recently published research reports suggest, however, that stem cell biology and paradigm may be more complex than originally postulated; and have suggested instead that rather than referring to a discrete cellular entity or stage of development, the term “stem cell” more accurately refers to a biological function that can be induced in many distinct types of cells, even differentiated cells. This concept and view has been presuasively presented by H. M. Blau, T. R. Braelton and J. M. Weimann in their published review entitled “The Evloving Concept Of A Stem Cell:Entity Or Function” [Cell 105:829-841 (2001)], the text of which is expressly incorporated by reference herein. A pertinent restatement of the Blau et al. evolving concept of a stem cell is reproduced below for the benefit and consideration of the reader.

The evolving concept of a functional stem cell definition and sytem is based on the empirical demonstration that stem cells in adults can first residue in one tissue and then contribute to repair or development of another tissue. This migration phenomenon and contribution effect reveals a previously unrecognized degree of plasticity in stem cell function. Moreover, it now appears that cell fate changes are a natural property of stem cells and may be involved in ongoing physiological repair of tissue damage throughout life. Although the number of reported instances are still relatively few, the evidence of this unexpected finding suggests that the concept of stem cells is in a state of flux; and that the commonly held view of a tissue-specific adult stem cell may need to be expanded.

As part of this concept, identifiable adult stem cells may not only act locally in the tissues in which they residue in vivo, but also may be recruited out of the circulation and enlisted in regeneration of diverse tissues at distal sites. Some stem cells can transit through the circulation, which is envisioned as a ‘stem cell highway’ with access to all organs of the body. Thus, as empirically reported, bone marrow derived cells can enter different organs including the heart, brain, skeletal muscle and liver. Also “homing signals” may result from local tissue damage and influence the migration of stem cells (in a manner reminiscent of white blood cell homing). Growth factors in the local areas then induce stem cells to participate in the function of the organ they enter. Thus, the microenvironment, including reactive contact with surrounding cells, the extracellular matrix, the local milieu, as well as growth and differentiation factors are deemed to play a key role in determining a stem cell's function.

Moreover, within organs such as the brain, liver and muscle, it is well known that there is a resident pool of identifiable stem cells, long thought to be dedicated exclusively to the repair of the tissue in which they reside. However, since the stem cells can enter an organ via the circulation, these newly entered stem cells can either contribute to the existing pool of stem cells existing previously within that organ; or directly generate differentiated cells themselves in the local environment.

Taken to an extreme, therefore, even highly specialized cell types in tissues may be capable of reversing their differentiated state and contributing to the pool of functional stem cells—as recent empirical studies have shown using multinucleated muscle cells and differentiated CNS cells. Thus, according to the functional concept of a stem cell, at least some identifiable stem cells in adult tissues are capable of movement between tissues; and cell fate changes make it clear that at least a subset of stem cells may alter their function in a manner that is highly plastic and amenable to change given the appropriate microenvironment. For these reasons, the overall conceptual proposal is that a stem cell most accurately refers to a biological function that can be induced in many distinct types of cells, even differentiated cells.

In addition, recent findings presented only in abstract form by Reyes et al. (American Society of Hematology, 43rd Annual Meeting, December, 2001, “Engraftment and Tissue Specific Differentiation of Multipotent Adult Progenitor Cells from Human Marrow in Epithelium, the Hematopoietic System and Endothelium In Vivo”) and by Wernet et al. (American Society of Hematology 43rd Annual Meeting, December 2001, “Detection of Unrestricted Multipotential Stem Cells in Human Cord Blood”) indicate that there may be cells in adult tissues, ranging from cord blood to bone marrow and other adult organs, that are truly pluripotent. These identified types of adult stem cells can form tissues derived from the ectoderm, endoderm, and mesoderm, previously thought to be a property only of embryonic stem cells. The potential—that it is these cells in each organ, and not cell trafficking, that can be responsible for the complex regeneration of tissues in multiple organs—remains to be fully validated. These cells also appear to be precursors to the other Orders described in this application; and as such have utility in this invention as identifiable types of stem cells.

The present invention takes into account each of these concepts and alternative definitions of stem cells, which can alter their function in a plastic and dynamic manner. The Blau et al. proposal and concept appears to have substantial merit and empirical evidence in support of its views. Similarly, the Reyes et al. and Wernet et al. reseach data provides emprical support for their respective ideas. For these reasons, the present invention does not rely exclusively upon the classical paradigm and system model; and allows for the presently evovling concepts and views that stem cells and their offspring generational descendant cells may be identified, isolated and employed within the instant therapeutic treatment methods on the basis of biological function, rather than upon an irrevesible progression along a well defined pathway concluding in a terminally differentiated cell.

Nevertheless, for the sake of clarity of description, a focused presentation, as well as for an unambiguous terminology, the detailed disclosure which follows herein will describe the classical paradigm and system most commonly understood, accepted and employed today.

### IV. The Individual Categories (Orders) of Identiable Stem Cells

The true source or origins of the chosen category (or Order) of stem cells does not meaningfully matter so long as the cells employed are biocompatible with the intended host recipient. All that is required for practicing the present invention is that a plurality of identifiable stem cells (of mammalian origin), and/or their immediate progeny and/or offspring descendant cells, be available as viable cells for implantation purposes.

It is both preferred and desirable that the chosen category of stem cells be a stable culture of cells maintained in-vitro; be a partially- or completely-purified culture of cells having a common cell ancestor; and be in a active or mitotic stage of existence. It is also expected that the best biological compatibility between the inoculum of cells and host recipient will exist when both are of identical or very similar species/subspecies origin. Thus, stem cells of human origin are preferred for use with human recipient subjects; murine sources of stem cells are desirable for use with rats and mice; and other sources of stem cells from the various mammalian species (e.g., pigs, horses, cows, rabbits, dogs & cats) are preferred for use with each of these mammalian types specifically.

### Embryonic Stem Cells and Their Offspring: Their Progenitor Cells, Their Lineage-Committed Cells and Their Partially-Differentiated Cells

Embryonic stem cells have the ability to divide for indefinite periods in culture and to give rise to specialized cells. They are often described in the context of a normal human development—which begins when a sperm fertilizes an egg and creates a single cell that has the potential to form an entire organism. The fertilized egg is totipotent, meaning that its potential is total. In the first hours after fertilization, this cell divides into identical totipotent cells. Thus any one of these cells, if placed into a uterus, has the potential to develop into a fetus.

Approximately four days after fertilization, and after several cycles of cell division, these totipotent cells begin to specialize and form a hollow sphere of cells, a blastocyst. The blastocyst has an outer layer of cells; and inside the hollow sphere, there is another cluster of cells termed the inner cell mass.

The outer layer of cells will subsequently form the placenta and the other external supporting tissues needed for fetal development in the uterus. The inner cell mass cells will go on to form virtually all of the tissues of the human body. Although the inner cell mass can form virtually every type of cell found in the human body, they cannot form a whole organism—primarily because they are unable to give rise to the placenta and supporting tissues necessary for development in the uterus.

These inner cell mass cells are termed “pluripotent”—they can give rise to many types of cells, but not all types of cells necessary for fetal development. Thus, because their potential is not total, they are not “totipotent” as such, and they are not capable of becoming living embryos. In fact, if an inner cell mass cell were placed into a woman's uterus, it could not and would not develop into a fetus.

The pluripotent stem cells undergo further development into progeny cells which multiple in great numbers and, in turn, later become lineage-committed and subsequently give rise to cells that are differentiated and have a particular function. Examples of this cell development lineage scheme include stem cells which give rise to red blood cells, to white blood cells and to platelets; and those stem cells that over time give rise to the various types of skin cells. These more limited progeny cells are thus often called “multipotent” progenitor cells.

Embryonic stem cells have been identified and isolated from a broad range of mammalian genera and species. The recognized cell deposits and natural sources today include mouse, rats, and other rodents; dogs, cats and rabbits; monkeys of various kinds; as well as humans.

The first documentation of the isolation of embryonic stem cells from human blastocysts was in 1994 [Bongso et al., Hum. Reprod. 9: 2110-2117 (1994)]. Since then, techniques for deriving and culturing human ES cells have been refined [Reubinoff et al., Nat. Biotechnol. 15: 399-404 (2000); Thomson et al., Science 282: 1145-1147 (1998)]. The ability to isolate human ES cells from blastocysts and grow them in culture seems to depend in large part on the integrity and condition of the blastocysts from which the cells are derived. In general, blastocysts with a large and distinct inner cell mass tend to yield ES cultures most efficiently [Bongso A., Handbook on Blastocyte Culture, Sydney Press Review, Singapore, 1999].

Day-5 blastocysts are often used to derive ES cell cultures in vitro. A normal day-5 human embryo in vitro consists of 200 to 250 cells. Most of the cells comprise the trophectoderm, the outer layer of cells surrounding the blastocyst. For deriving ES cell cultures, the trophectoderm is removed, either by microsurgery or immunosurgery (in which antibodies against the trophectoderm help break it down, thus freeing the inner cell mass). At this stage, the inner cell mass is composed of only 30 to 34 cells [Bongso et al., Asst. Reprod. Rev. (1999)].

The in vitro conditions for growing a human embryo to the blastocyst stage can vary. See, for example, Bongso et al., Cell Biol. Int. 18: 1181-1189 (1994); Bongso et al., Assit. Reprod. Rev. 5: 106-114 (1995); Fong et al., Hum. Reprod. 13: 2926-2932 (1998); DeVos et al., Cells Tissues Organs. 166: 220-227 (2000); Jones et al., Fetil. Steril. 70: 1022-1029 (1998); Sathananthan, A. H., Hum. Cell. 10: 21-38 (1997); Trounson et al., Handbook of In-Vitro Fertilization, CRC Press, 2000; Trounson et al., Reproduction 121: 51-57 (2001). However, once the inner cell mass is obtained from either mouse or human blastocysts, the techniques for growing ES cells are similar.

Embryonic stem cells as a category have also been compared and distinguished from both ‘embryonic germ cells’ and ‘embryonal carcinoma cells’ in meaningful terms. A comparison of the cell markers which are typically used to identify and separate them is given by Table 1 below.

TABLE 1
Comparison of Mouse, Monkey, and Human Pluripotent Stem Cells*

| | | Monkey ES | | Human | Human |
|---|---|---|---|---|---|
| Marker Name | ES/EG cells | cells | Human ES cells | EG cells | EC cells |
| SSEA-1 | + | − | − | + | − |
| SSEA-2 | − | + | + | + | + |
| SEA-4 | − | + | + | + | + |
| TRA-1-60 | − | + | + | + | + |
| TRA-1-81 | − | + | + | + | + |
| Alkaline | + | + | + | + | + |
| phosphatase | | | | | |
| Oct-4 | + | + | + | Unknown | + |
| Telomerase | + ES,EC | Unknown | + | Unknown | + |
| activity | | | | | |
| Feeder-cell | ES, EG, some | Yes | Yes | Yes | Some; relatively |
| dependent | EC | | | | low clonal |
| | | | | | efficiency |
| Factors which aid | LIF and other | Co-culture with | Feeder cells + | LIF, bFGF, | Unknown; low |
| in stem cell self- | factors that act | feeder cells; | serum; feeder | forskolin | proliferative |
| renewal | through pg 130 | other promoting | layer + serum- | | capacity |
| | receptor and can | factoes have not | free medium + | | |
| | substitute for | been identified | bFGF | | |
| | feeder layer | | | | |
| Growth | Form tight, | Form flat, loose | Form flat, loose | Form | Form flat, loose |
| characteristics in | rounded, multi- | aggregates; can | aggregates; can | rounded, | aggregates; can |
| vitro | layer clumps; | form EBs | form EBs | multi-layer | form EBs |
| | can from EBs | | | clumps; can | |
| | | | | form EBs | |
| Teratoma | + | + | + | + | + |
| formation in vivo | | | | | |
| Chimera | + | Unknown | + | − | + |
| formation | | | | | |

KEY
ES cell = Embryonic stem cell
EG cell = Embryonic germ cell
EC cell = Embryonal carcinoma cell
SHEA = Stage-specific embryonic entigen
TRA = Tumor rejection antigen-1
LIF = Leukemia inhibitory factor
bFGF = Basic fibroblast growth factor
EB = Ebryoid bodies
*Reproduced from “The Human Embryonic Stem Cell And The Human Embroynic Germ Cell”, Chapter 3, in STEM CELLS, National Institutes of Health, 2000.

### Lineage-Committment and Partial-Differentiation of Cells Derived from Embryonic Stem Cells

The offspring of ES cells begin to undergo lineage-committment and differentiate if they are removed from feeder layers and grown in suspension cultures on a non-adherent surface. The human ES cells characteristically form embryoid bodies which, in the early stages, may be simple or cystic and filled with fluid. Although human embryoid bodies vary in their cellular content, many include cells that look like neurons and heart muscle cells [Itskovitz-Eldor et al., Mol. Med. 6: 88-95 (2000); Reubinoff et al., Nat. Biotechnol. 18: 399-404 (2000); Roach et al., Eur. Urol. 23: 82-87 (1993)].

After the human embryoid bodies form, they can be dissociated and replated in monolayer cultures which are then exposed to specific growth factors that influence further cell differentiation. Some growth factors induce cell types that would normally be derived from ectoderm in the embryo; these include retinoic acid, epidermal growth factor (EGF), and and bone growth factor (BGF). Other growth factors, such as activin-A and transforming growth factor-beta 1 (TGF-β-1), trigger the differentiation of mesodermally-derived cells. Two other factors, hepatocyte growth factor (HGF) and nerve growth factor (NGF), promote differentiation into all three germ layers, including the endoderm.

When these eight growth factors were added individually to cell cultures derived from embryoid bodies, the cells differentiated into 11 cell types that represented all three germ layers. The identity of the differentiated human embryoid body derived cells was determined by their morphology, growth characteristics and expression of messenger RNA (mRNA) for specific markers [Shamblott et al., Proc. Natl. Acad. Sci. USA 95: 13726-13731 (1998)].

It also has been noted that human embryoid body-derived cells can and will differentiate spontaneously into many kinds of cells without the addition of growth factors. However, the addition of merely one of (a number of different growth factors) resulted in cultures that were more likely to be populated by only one or two types of differentiated cells, as measured by mRNA transcripts expressed by the cells. Thus, human embryoid body-derived cultures treated with bFGF differentiated largely into epidermal epithelial cells that express keratin, a protein in skin. Similarly, cells in activin-A-treated culture media formed muscle cell-like, syncytium-fused, multinucleated populations of cells that express the enzyme muscle-specific enolase. Also, cultures treated with retinoic acid differentiated into cells that resemble neurons and express neurofilament H. However, the same growth factor typically induced the expression of multiple markers; and none of the resulting cell populations was homogeneous [Shamblott et al., Proc. Natl. Acad. Sci. USA 95: 13726-13731 (1998)].

Spontaneous differentiation of human ES cells into hematopoietic cells, which form all the lineages of blood cells, is rare in vitro. However, by co-culturing human ES cells with mouse bone marrow stromal cells (irradiated to prevent their replication) in growth medium that contains fetal bovine serum, but no added growth factors, the cells differentiate to form what appear to be hematopoietic precursor cells. The partly differentiated cells express CD34, a marker for blood cell precursors. If these partly differentiated human ES cells are replated under conditions that allow them to form colonies of hematopoietic cells, they differentiate into erythroid cells, macrophages, granulocytes, and megakaryocytes [Odorico et al., Stem Cells 19: 193-204 (2001)].

An abundance of additional knowledge and guidance information regarding embryonic stem cells, their progeny progenitor cells, and their lineage-committed descendant cells is provided by the published scientific and patent literature. Merely examplifying some of these are: “Stem Cells: A Primer”, National Institutes of Health, May, 2000; “The Human Embryonic Germ Cell”, Chapter 3 in Stem Cells, National Institutes of Health, 2000; NIH Human Embryonic Stem Cell Registry, National Institutes of Health, 2001. Also, the patent literature offers much information, insight and guidance concerning embryonic stem cells. Examplifying these are U.S. Pat. Nos. 5,843,780; 6,200,806; 5,639,618; 6,146,888; and 6,280,718—the texts of which are each expressly incorporated by reference herein.

### Adult Stem Cell Category 1: Hematopoietic Stem Cells and Their Offspring; Their Progenitor Cells, Their Lineage-Committed Cells and Their Partially-Differntiated Cells

A hematopoietic adult stem cell may be defined as a pluripotent cell which: (1) gives rise to progeny in all defined hematolymphoid lineages; and (2) provides additional numbers of cells which are capable of fully reconstituting a seriously immunocompromised host in all blood cell types by cell renewal.

Certainly, differentiated mammalian blood cells must provide for an extraordinarily diverse range of activities. The blood cells are divided into several lineages, including lymphoid, myeloid and erythroid. The lymphoid lineage; comprising B cells and T cells, provides for the production of antibodies, regulation of the cellular immune system, detection of foreign agents in the blood, detection of cells foreign to the host, and the like. The myeloid lineage, which includes monocytes, granulocytes, megakaryocytes, as well as other cells, monitors for the presence of foreign bodies in the blood stream, provides protection against neoplastic cells, scavenges foreign materials in the blood stream, produces platelets, and the like. The erythroid lineage provides the red blood cells, which act as oxygen carriers.

Despite the diversity of the nature, morphology, characteristics and function of the blood cells, it is presently believed that there is a single type of hematopoietic stem cell which is capable of self regeneration and which, after exposure to growth and other environmental factors, becomes dedicated to a specific lineage pathway and pattern of cell differentiation.

The high turnover of mammalian blood cells requires a in-vivo supply of hematopoietic stem cells that are able to give rise to the various blood cell lineages. The immediate progeny of the hematopoietic stem cell are called progenitor cells; and these progenitor cells are capable of giving rise to various cell types within one or more specific lineage pathways—i.e., the erythroid, myeloid and lymphoid lineages. Overall, the hematopoietic stem cell and its progenitor cell populations constitute only a small percentage of the total number of cells in the bone marrow, fetal liver, etc. However, these populations are of immense value because of their ability to reproduce the entire hematopoietic system in a living mammal.

A number of methods have been described in the published literature for the purification or enrichment of hematopoietic stem cell and progenitor cell populations. There is significant interest in these methods because hematopoietic progenitors have a number of clinical uses. Progenitor cell transplantation is currently used in conjunction with chemotherapy and radiation for the treatment of leukemia, breast cancer and other tumors. There is also interest in the use of hematopoietic progenitor cells as a vehicle for gene therapy. Although not yet proven in the clinic, the longevity of hematopoietic stem cells and the dissemination of their progeny in the vasculature are desirable characteristics. A number of vectors, including several retrovirus and adenovirus based constructs, that can transfect hematopoietic stem cells have been described.

Proteins and other cell surface markers found on hematopoietic stem cell and progenitor cell populations are of great interest, as they are useful in preparing reagents for identification, separation and isolation of these populations and in the further characterization of these important cells. Some markers are now known that can be used in the identification and separation (positive and negative) of stem cells, such as the CD 34 antigen, which is found on stem cells but not on mature blood cells.

The published scientific and patent literature provides an abundant quantum of useful knowledge and guidance information. For example, U.S. Pat. Nos. 5,643,741; 5,716,827, 5,843,633, and 5,061,620 describe substantially homogenous human hematopoietic stem cells and the manner of obtaining such cells. Stromal cell-associated hematopoiesis is described by Paul et al. (1991) Blood 77:1723-1733. The phenotype of stem cells with rhodamine staining is discussed in Spangrude and Johnson (1990) P.N.A.S. 87: 7433-7437. Cell surface antigen expression in hematopoietic is discussed in Strauss et al. (1983) Blood 61: 1222-1231 and Sieff et al. (1982) Blood 60: 703-713. Descriptions of pluripotential hematopoietic cells are found in McNiece et al. (1989) Blood 74: 609-612 and Moore et al. (1979) Blood Cells 5: 297-311. Characterization of a human hematopoietic progenitor cell capable of forming blast cell-containing colonies in vitro is found in Gordon et al. (1987) J. Cell. Physiol. 130: 150-156 and Brandt et al. (1988) J. Clin. Invest. 82: 1017-1027. The use of progenitor cells in transplantation is discussed in To et al., in Progenitor Threshold in Transplantation (ISBN 1-880854 17-1), pp. 15-20. All of these publications are expressly incorporated by reference herein.

In addition, the U.S. patent literature provides the following: methods for culturing human hematopoietic stem cells in vitro [U.S. Pat. Nos. 5,436,151; 5,460,964; and 5,605,822]; methods for regulating the specific lineages of cells produced in a human hematopoietic cell culture [U.S. Pat. No.,5,635,386]; methods for purifying a population of cells enriched for hematopoietic stem cells [U.S. Pat. No. 5,665,557]; a method and compositions for the ex-vivo replication of human hematopoietic stem cells [U.S. Pat. No. 5,670,351]; method for producing a highly enriched population of hematopoietic stem cells [U.S. Pat. Nos. 5,814,440 and 5,681,559]; peripheralization of hematopoietic stem cells [U.S. Pat. Nos. 5,843,438, 5,695,755 and 5,824,304]; and the expansion of human hematopoietic progenitor cells in a liquid medium [U.S. Pat. No. 5,744,361]. The texts of these issued patents are each expressly incorporated by reference herein.

### The Hematopoietic Stem Cell

A useful description of and means for identifying human hematopoietic stem cells and its progenitor cells is provided by U.S. Pat. No. 5,643,741, of which a pertinent part is restated below.

The hematopoietic stem cells are characterized both by the presence of markers associated with specific epitopic sites identified by antibodies and the absence of certain markers as identified by the lack of binding of certain antibodies. It is not necessary that selection is achieved with a marker specific for stem cells. By using a combination of negative selection (removal of cells) and positive selection (isolation of cells), a substantially homogeneous hematopoietic stem cell composition can be achieved.

The hematopoietic stem cells are characterized by being for the most part CD34+, CD3−, CD7−, CD8−, CD10−, CD14−, CD15−, CD19−, CD20−, CD33−, and Thy-1+. A highly stem cell concentrated cell composition is CD34+, CD10−, CD19− and CD33−, more particularly in addition CD3− and CD8−, preferably in addition Thy-1+. The CD3−, CD8−, CD10−, CD19−, CD20−, and CD33−, will be referred to as Lin−. The CD10/19/20 markers are associated with B-cells, CD3/4/8 markers are associated with T-cells, CD 14/15/33 cell markers are associated with myeloid cells. The Thy-1 marker is absent on human T-cells. Also, for human CD34+, rhodamine 123 can divide the cells into high and low subsets. [See Spangrude, (1990) Proc. Natl. Acad. Sci. 87: 7433 for a description of the use of rhodamine 123 with mouse stem cells.] Preferably the cells are rhodamine low.

### Isolation of Hematopoietic Stem Cells

In order to obtain hematopoietic stem cells, it is necessary to isolate the rare pluripotent stem cell from the other cells in bone marrow or other hematopoietic source. Initially, bone marrow cells may be obtained from a source of bone marrow, e.g., iliac crests, tibiae, femora, spine, or other bone cavities. Other sources of human hematopoietic stem cells include embryonic yolk sac, fetal liver, fetal and adult spleen, blood, including adult peripheral blood and umbilical cord blood.

For isolation of bone marrow from fetal bone or other bone source, an appropriate solution may be used to flush the bone, which solution will be a balanced salt solution, conveniently supplemented with fetal calf serum or other naturally occurring factors, in conjunction with an acceptable buffer at low concentration, generally from about 5-25 mM. Convenient buffers include Hepes, phosphate buffers, lactate buffers, etc. Otherwise bone marrow may be aspirated from the bone in accordance with conventional ways.

Morphologic evaluation of the 34+ Thy+Lin− cells indicates that the multipotent stem cells. are of medium size. Light scatter evaluation shows that stem cells have a blast cell profile with low side scatter. These observations indicate that the stem cells have a unique density profile. It has been found that the low density fractions from density fractionated human bone marrow are enriched for CD34+ Thy+Lin− cells.

Various techniques may be employed to separate the cells by initially removing cells of dedicated lineage. Monoclonal antibodies are particularly useful for identifying markers (surface membrane proteins) associated with particular cell lineages and/or stages of differentiation. The antibodies may be attached to a solid support to allow for crude separation. The separation techniques employed should maximize the retention of viability of the fraction to be collected. For “relatively crude” separations (that is, separations where up to 10%, usually not more than about 5% and preferably not more than about 1% of the total cells present having the marker may remain with the cell population to be retained) various techniques of different efficacy may be employed. The particular technique employed will depend upon efficiency of separation; cytotoxicity of the methodology; ease and speed of performance; and necessity for sophisticated equipment and/or technical skill.

Procedures for stem cell separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique. Techniques providing accurate separation include fluorescence activated cell sorters, which can have varying degrees of sophistication, e.g., a plurality of color channels, low angle and obtuse light scattering detecting channels, impedance channels, etc.

One procedure which may be used is in a first stage after incubating the cells from the bone marrow for a short period of time at reduced temperatures, generally about 4° C., with saturating levels of antibodies specific for a particular cell type, e.g., CD3 and CD8 for T-cell determinants. The cells are then washed with a fetal calf serum (FCS) cushion. The cells may then be suspended in a buffer medium as described above and separated by means of the antibodies for the particular determinants, using various proteins specific for the antibodies or antibody-antigen complex.

Conveniently, the antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Thus, any technique may be employed which is not unduly detrimental to the viability of the remaining cells.

After substantial enrichment of the cells lacking the mature cell markers (generally by at least about 50% and preferably at least about 70%) the cells may now be separated by a fluorescence activated cell sorter (“FACS”) or other methodology having high specificity. Multi-color analyses may be employed with the FACS which is particularly convenient.

The cells may be separated on the basis of the level of staining for the particular antigens. In a first separation, starting with at least about 1-3×10¹⁰ cells, the antibody for CD34 may be labeled with one fluorochrome, while the antibodies for the various dedicated lineages may be conjugated to a different fluorochrome. Fluorochromes which may find use in a multi-color analysis include phycobiliproteins, e.g., phycoerythrin and allophycocyanins, fluorescein, Texas red, etc. While each of the lineages may be separated in a separate step, desirably the lineages are separated at the same time as one is positively selecting for CD34 or equivalent marker. Generally, the number of cells obtained will be fewer than about 1% of the original cells, generally fewer than about 0.5% and may be as low as 0.2% or less.

The cells may then be further separated by positively selecting for Thy+, where the cells will generally be fewer than 0.5% of the original cells, generally in the range of 0.01-0.5%. The cells may be selected against dead cells, by employing dyes associated with dead cells (propidium iodide, LDS). Desirably, the cells are collected in a medium comprising 2% fetal calf serum. Other techniques for positive selection may be employed, which permit accurate separation, such as affinity columns, and the like. The method should permit the removal to a residual amount of less than about 20%, preferably less than about 5%, of the non-stem cell populations.

The CD34+ Lin− and the CD34+ Lin− Thy-1+ have low side scatter and low forward scatter profiles by FACS analysis. CYTOSPIN preparations show the stem cell to have a size between mature lymphoid cells and mature granulocytes. Cells may be selected based on light-scatter properties as well as their expression of various cell surface antigens.

### Lineage-Committted Cells and Partially-Differentiated Cells

Compositions having greater than 90% (usually greater than about 95%) of human hematopoietic stem cells may be isolated—where the desired where the desired stem cells are identified by being CD34+, Lln− and Thy-1+. These cells are able to provide for cell regeneration and development of members of all of the various hematopoietic lineages.

The human hematopoietic stem cells provide for production of myeloid cells and lymphoid cells in appropriate cultures; cultures providing hydrocortisone for production of myeloid cells (associated with Dexter-type cultures); and B lymphocytes in cultures lacking hydrocortisone, (associated with Whitlock-Witte type cultures). In each of the cultures, mouse or human stromal cells are provided, which may come from various strains. AC3 or AC6, stromal cells derived from mouse or human fetal bone marrow by selection are able to maintain human stem cells. The medium employed for the culturing of the cells is conveniently a defined enriched medium, such as IMDM (Iscove's Modified Dulbecco's Medium), a 50:50 mixture of IDM and RPMI, and will generally be composed of salts, amino acids, vitamins, 5.times.10.sup.−5 M 2-ME, streptomycin/penicillin and 10% fetal calf serum, and may be changed from time to time, generally at least about once to twice per week. Particularly, by transferring cells from one culture with hydrocortisone, to the other culture without hydrocortisone, and demonstrating the production of members of the different lineages in the different cultures, the presence of the stem cell and its maintenance is supported. In this manner, one may identify the production of both myeloid cells and B-cells.

To demonstrate differentiation to T-cells, one may isolate fetal thymus and culture the thymus for from 4-7 days at about 25° C., so as to substantially deplete the lymphoid population of the fetal thymus. The cells to be tested are then microinjected into the thymus tissue, where the HLA of the population which is injected is mismatched with the HLA of the thymus cells. The thymus tissue may then be transplanted into a scid/scid mouse as described in EPA 0 322 240, particularly transplanting in the kidney capsule.

For red blood cells, one may use conventional techniques to identify BFU-E units, for example methylcellulose culture (Metcalf (1977) In: Recent Results in Cancer Research 61. Springer-Verlag, Berlin, pp 1-227) demonstrating that the cells are capable of developing the erythroid lineage.

In identifying myeloid and B-cell capability, conveniently, the population to be tested is introduced first into a hydrocortisone containing culture and allowed to grow for six weeks in such culture. The medium employed will comprise a 50:50 mixture of RPMI 1640 and IMDM containing 10% FCS, 10% horse serum, streptomycin/penicillin, glutamine and 5.times.10.sup.−7 M hydrocortisone. During the six week period it would be anticipated that in the absence of progenitor cells, all of the mature cells would die. If at the end of six weeks, myeloid cells are still observed, one may conclude that there is a progenitor cell which is providing for the continuous differentiation to myeloid cells. At this time, one may then change the medium, so that the medium now lacks hydrocortisone, to encourage the growth of B-cells. By waiting 3-4 weeks and demonstrating the presence of B-cells by FACS analysis, one may conclude that the progenitor cells which previously were capable of producing myeloid cells are also capable of producing B-cells. Human hematopoietic cells grown in the presence of hydrocortisone can be maintained for at least four months. Similarly, human hematopoietic cells grown in the absence of hydrocortisone contain B lymphocytes (CD19+), as well as myelomonocytic cells for at least four months. From these cultures, one may sort for CD34+ Lin−, CD34+ Thy+, Thy+Lin−, or CD34+ Thy+Lin−, which should provide a composition substantially concentrated in the progenitor cell. The CD34+ Lin−, CD34+ Thy+, Thy+Lin−, or CD34+ Thy+Lin− cells obtained from these cultures can give rise to B-cells, T-cells and myelomonocytic cells.

### Adult Stem Cell Category 2: Mesenchymal Stem Cells and Their Offspring; Their Progenitor Cells, Their Lineage-Committed Cells and Their Partially-Differentiated Cells

Mesenchymal stem cells (MSCs) are the formative pluripotent embryonic-like cells found in bone marrow, blood, dermis, and periosteum; and are capable of differentiating into specific types of mesenchymal or connective tissues including adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues. Although these mesenchymal stem cells are normally present at very low frequencies in bone marrow, a number of processes for isolating, purifying, and mitotically expanding the population of these cells in tissue culture are presently known conventionally and used commercially.

Mesenchymal stem cells are defined as cells which are not terminally differentiated; which can divide without limit; and divide to yield daughter cells that are either stem cells or are progenitor cells which in time will irreversibly differentiate to yield a phenotypic cell. Those mesenchymal stem cells which give rise to many cell types are called pluripotent cells.

Chondro/osteoprogenitor cells, which are bipotent cells with the ability to differentiate into cartilage or bone, can be isolated from bone marrow (for example, as described in Owen, J. Cell Sci. Suppl. 10: 63-76 (1988) and in U.S. Pat. No. 5,226,914). These cells led Owen to postulate the existence of pluripotent mesenchymal stem cells, which were subsequently isolated from muscle (Pate, et al., Proc. 49th Ann. Sess. Forum Fundamental Surg. Problems 587-589 (Oct. 10-15, 1993); heart (Dalton et al., J. Cell Biol. 119: R202 (March 1993)); and granulation tissue (Lucas et al., J. Cell Biochem. 122: R212 (March 1993)). Pluripotency is demoonstrated using a non-specific inducer, dexamethasone (DMSO), which elicits differentiation of the stem cells into chondrocytes (cartilage), osteoblasts (bone), myotubes (muscle), adipocytes (fat), and connective tissue cells.

Unfortunately, although it is highly desirable to have stem cells which are easily obtained by a muscle biopsy, cultured to yield large numbers, and can be used as a source of connective tissue, or marrow, or chondrycytes, or osteoblasts, or myocytes; there is no known specific inducer of the mesenchymal stem cells that yields only cartilage. In vitro studies in which differentiation is achieved yields a mixture of cell types. For example, studies described in U.S. Pat. Nos. 5,226,914 and 5,197,985 in which the cells were absorbed into porous ceramic blocks and implanted were shown to yield primarily bone. Other studies using bone morphogenic protein-2 (rhBMP-2) in-vivo always yielded an endochondral bone cascade: that is, cartilage is formed first, but this cartilage hypertrophies; is then invaded by vasculature and osteoblasts; and is eventually replaced by bone complete with marrow (Wozney, Progress in Growth Factor Research 1: 267-280 (1989)). Studies testing rhBMP-2 on the mesenchymal stem cells in vitro produced mixtures of differentiated cells, although cartilage predominated (Dalton et al., J. Cell Biol. 278: PZ202 (February 1994)). Incubation of mesenchymal cell cultures with insulin led to a mixed myogenic and adipogenic response, while incubation with insulin-like growth factors I or II led to a primarily myogenic response (Young et al., J. Cell Biochem. 136: CD207 (April 1992)). Also U.S. Pat. Nos. 4,774,322 and 4,434,094 report the isolation of a factor that induces an osteogenic response in vivo or cartilage formation in vitro when mixed with muscle cells.

Overall, the U.S. patent literature provides a broad range of information and knowledge concerning mesenchymal stem cells, their immediate progeny cells, and their descendant, lineage-directed, differentiated cells. The isolated mesenchymal stem cells and their individual mammalian sources are described in full by U.S. Pat. Nos. 5,827,735; 5,486,359; 5,591,625; and 6,214,639. Methods for isolating and purifying mesenchymal stem cells from a variety of naturally occurring sources are described by U.S. Pat. Nos. 5,197,985; 5,226,914; 5,908,782; 5,965,436; and 6,261,549. Ligands and methods for controlling and directing the lineage commitment and differentiation of cells descending from mesenchymal stem cells are described by U.S. Pat. Nos. 5,811,094; 5,827,740; 5,942,225; 6,022,540, 6,149,906; 6,322,784; 5,719,058; 5,856,186; 5,908,784; 6,214,639; 6,248,587; and 6,338,942. The range and variety of intended commercial applications and/or therapeutic uses for mesenchymal stem cells and their offspring cells is exemplified by U.S. Pat. Nos. 5,700,289; 5,716,616; 5,719,058; 5,811,094; 5,837,539; 6,010,696; 6,149,906; 6,174,333; 6,225,119; 6,239,157; 6,255,112; 6,281,012; and 6328960. The descriptive text for each of these issued U.S. patents is expressly and individually incorporated by reference herein.

### EXAMPLES OF THE ISOLATION AND PURIFICATION OF HUMAN MESENCHYMAL STEM CELLS

The human mesenchymal stem cells can be derived, for example, from bone marrow, blood, dermis, periosteum, or even adipose tissues. When obtained from bone marrow, this can be marrow from a number of different sources, including: from plugs of femoral head cancellous bone pieces; from patients with degenerative joint disease during hip or knee replacement surgery; or from aspirated marrow obtained from normal donors and oncology patients who have marrow harvested for future bone marrow transplantation.

The harvested marrow is then prepared for cell culture. The isolation process involves the use of a specially prepared medium that contains agents which allow for not only mesenchymal stem cell growth without differentiation, but also for the direct adherence of only the mesenchymal stem cells to the plastic or glass surface of the culture vessel. By creating a medium which allows for the selective attachment of the desired mesenchymal stem cells which were present in the mesenchymal tissue samples in very minute amounts, it then became possible to separate the mesenchymal stem cells from the other cells (i.e., red and white blood cells, other differentiated mesenchymal cells, etc.) present in the mesenchymal tissue of origin.

As a result, a variety of processes have been developed for isolating and purifying human mesenchymal stem cells from tissue prior to differentiation; and then for culture expanding the mesenchymal stem cells. The objective of such manipulation is to greatly increase the number of mesenchymal stem cells; and later to utilize these cells to redirect and/or reinforce the body's normal reparative capacity. The mesenchymal stem cells are preferably expanded to great numbers and can be applied to areas of connective tissue damage to enhance or stimulate in vivo growth for regeneration and/or repair, to improve implant adhesion to various prosthetic devices through subsequent activation and differentiation.

Also, various procedures are contemplated for transferring, immobilizing, and activating the culture-expanded, purified mesenchymal stem cells at the site for repair, implanation, etc., including injecting the cells at the site of a skeletal defect, incubating the cells with a prosthesis and implanting the prosthesis, etc. Thus, by isolating, purifying and greatly expanding the number of cells prior to differentiation and then actively controlling the differentiation process by virtue of their positioning at the site of tissue damage or by pretreating in vitro prior to their transplantation, the culture-expanded, mesenchymal stem cells have been utilized for various therapeutic purposes (such as to alleviate cellular, skeletal dysplasias, cartilage defects, ligament and tendon injury and other musculoskeletal and connective tissue disorders).

Several media have been prepared which are particularly well suited to the desired selective attachment and-are often referred to herein as “Complete Media” when supplemented with serum as described below. One such medium is an augmented version of Dulbecco's Modified Eagle's Medium-Low Glucose (DMEM-LG), which is well known and readily commercially available. Other in-vitro culture media formulations are described by U.S. Pat. Nos. 5,942,225; 5,908,782; 5,919,702; 5,795,781; 6,174,333; 6,030,836; and 5,486,359.

### Lineage-Committed Cells and Partially-Differentiated Cells

Much of the information presented here is a pertinent restatement of the information provided by U.S. Pat. No. 5,942,225.

The capacity of undifferentiated progenitor progeny cells to enter discrete lineage pathways is referred to as the mesengenic process, and is diagrammatically represented in Flow Scheme A. In the mesengenic process, MSCs are recruited to enter specific multi-step lineage pathways which eventually produce functionally differentiated tissues such as bone, cartilage, tendon, muscle, dermis, bone marrow stroma, and other mesenchymal connective tissues. For example, a detailed scheme for the differentiation pathway of bone forming cells is presented in Flow Scheme B. This lineage pathway map implies the existence of individual controlling elements which recruit the MSCs into the osteogenic lineage; promote pre-osteoblast replication; and direct step-wise differentiation all the way to the terminal stage osteocyte. Substantial work has been published that supports the view that each step of this complex pathway is controlled by different bioactive factors.

A similar lineage commitment diagram has been developed for chondrocyte differentiation, as shown by Flow Scheme C. Again, progression of each lineage pathway step is under the control of unique bioactive factors including, but not limited to, the family of bone morphogenetic proteins. Each modulator of the differentiation process, whether in bone, cartilage, muscle, or any other mesenchymal tissue, may affect the rate of lineage progression and/or may specifically affect individual steps along the pathway. That is, whether a cell is nascently committed to a specific lineage, is in a biosymetrically active state, or progresses to an end stage phenotype will depend on the variety and timing of bioactive factors in the local environment.

[image]

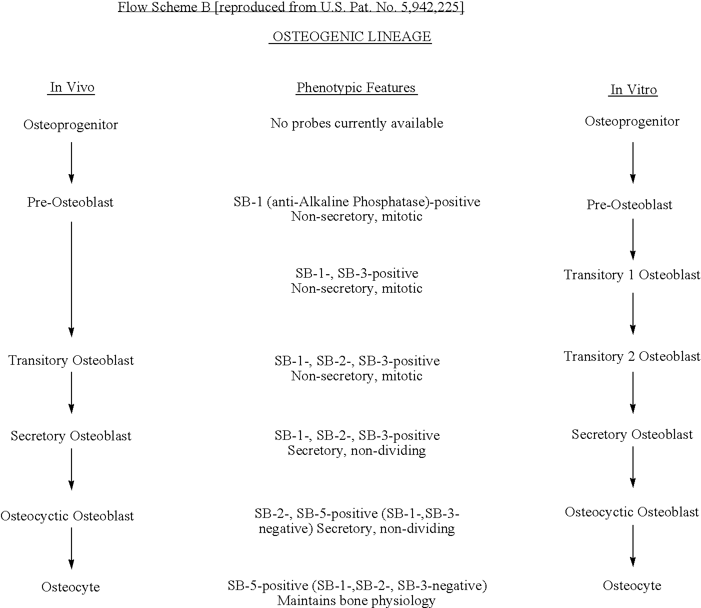

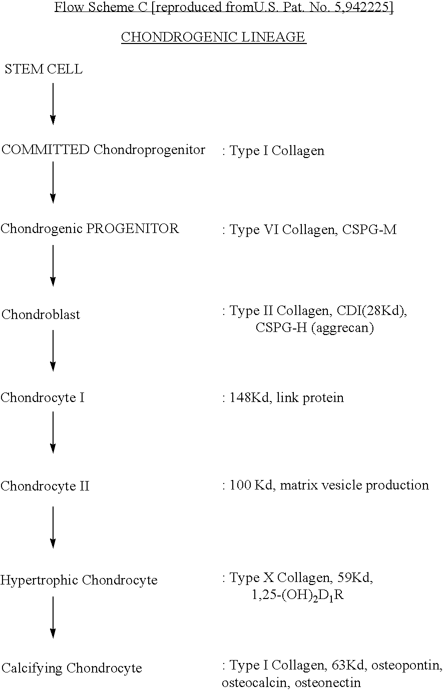

It is thus clearly indicated that under certain conditions, culture expanded mesenchymal stem cells have the ability to differentiate into bone, or cartilage, or any of the other types shown by Fow Scheme A. The environmental factors which influence the mesenchymal stem cells to differentiate into bone or cartilage cells appears, in part, to be the direct accessibility of the mesenchymal stem cells to growth and nutrient factors supplied by the vasculature; or to cells that are closely associated with the vasculature.

As a result, the isolated and culture expanded mesenchymal stem cells can be utilized, under certain specific conditions and/or under the influence of certain factors, commit to a specific lineage pathway and to differentiate and produce the desired cell phenotype needed for connective tissue repair or regeneration and/or for the implantation of various prosthetic devices. For example, using porous ceramic cubes filled with culture-expanded human mesenchymal stem cells, bone formation inside the pores of the ceramics has been generated after subcutaneous incubations in immunocompatible hosts.

Factors which stimulate osteogenesis (i.e., are osteoinductive) from isolated human mesenchymal stem cells in accordance with the invention are provided by several classes of molecules, including: bone morphogenic proteins, such as BMP-2 and BMP-3; growth factors, such as basic fibroblast growth factor (bFGF); glucocorticoids, such as dexamethasone; and prostaglandins, such as prostaglandin El. Furthermore, ascorbic acid and its analogs (such as ascorbic acid-2-phosphate) and glycerol phosphates (such as beta.-glycerophosphate) are effective adjunct factors for advanced differentiation, although alone they do not induce osteogenic differentiation.

Factors which have chondroinductive activity on human MSCs also are present in several classes of molecules, including: compounds within the transforming growth factor-beta (TGF-beta) superfamily such as (i) TGF-beta.1, (ii) Inhibin A, (iii) chondrogenic stimulatory activity factor (CSA) and (iv) bone morphogenic proteins, such as BMP-4; collagenous extracellular matrix molecules, including type I collagen, particularly as a gel; and vitamin A analogs such as retinoic acid.

Factors which have stromagenic inductive activity on human MSCs are also present in several classes of molecules, especially the interleukins, such as IL-1 alpha and IL-2.

Factors which have myogenic inductive activity on human MSCs are also present in several classes of molecules, especially cytidine analogs, such as 5-azacytidine and 5-aza-2′-deoxycytidine.

The effect of these modulating factors on human MSCs is disclosed here merely as illustrative examples. This disclosure is not an all-inclusive listing of potentially useful modulatory factors for inducing differentiation into a particular lineage, but merely illustrates the variety of compounds which have shown biologic activity for promoting the step-wise progression of mammalian mesenchymal stem cell differentiation along specific pathways.

### Adult Stem Cell Category 3: Neural Stem Cells and Their Offspring; Their Progenitor Cells, Their Lineage-Committed Cells and Their Partially-Differentiated Cells

Neural stem cells are uncommitted and undifferentiated cells that exist in many tissues of embryos and adult organisms. In embryos, blastocyst stem cells are the source of cells which differentiate to form the specialized tissues and organs of the developing fetus. In adults, specialized stem cells in individual tissues are the source of new cells, replacing cells lost through cell death due to natural attrition, disease, or injury. These stem cells may be used as substrates for producing healthy tissue where a disease, disorder, or abnormal physical state has destroyed or damaged normal tissue.

### Neural Stem Cells of Mammalian Origin

Neural stem cells (NSCs) are primordial, uncommitted cells that exist in the developing and even adult nervous system and are responsible for giving rise to the array of more specialized cells of the mature CNS. They are operationally defined by their ability (a) to differentiate into cells of all neural lineages (neurons—ideally of multiple subtypes, oligodendroglia, astroglia) in multiple regional and developmental contexts (i.e., be multipotent); (b) to self-renew (i.e., also give rise to new NSCs with similar potential); (c) to populate developing and/or degenerating CNS regions.

An unambiguous demonstration of monoclonal derivation is obligatory to the definition—that is, a single cell must possess these attributes. With the earliest recognition that rodent neural cells with stem cell properties coukld be propagated in culture and stably express foreign genes, gene therapists and restorative neurobiologists began to consider how such cells might be harnessed for therapeutic advantage as well as for a better understanding of cell developmental mechanisms.

Neural stem cells (NSCs) have been isolated from the mammalian embryonic, human neonatal and adult rodent CNS; and propagated in vitro by a variety of effective and safe means—both epigenetic (e.g., with mitogens such as epidermal growth factor or basic fibroblast growth factor); or genetic (e.g., with propagating genes such as vmyc or SV40 large T-antigen). Maintaining such NSCs in a proliferative state in culture media does not appear to subvert their ability to respond to normal developmental cues in vivo following transplantation; or to interact with host cells; or to differentiate into specific phenotypes appropriately. These extremely plastic NSCs migrate and differentiate in a temporally and regionally appropriate manner particularly following implantation into germinal zones throughout the brain. Once situated, they participate in normal development along the neuraxis, intermingling non-disruptively with endogenous progenitors, responding similarly to local microenvironmental cues for their phenotypic determination and appropriately differentiating into diverse neuronal and glial cell types. In addition, NSCs can express foreign genes (both reporter genes and therapeutic genes) in vivo and are capable of specific neural cell replacement in the setting of absence or degeneration of neurons and/or glia.

The growing interest in the potential of NSCs has been analogous to that in other Orders of adult stem cells. This interest derives from the realization that NSCs are not simply a substitute for fetal tissue in transplantation paradigms or simply another vehicle for gene delivery. Their basic biology, at least as revealed through the examination of cells, appears to endow them with a potential that other vehicles for gene therapy and repair do not possess. For example, that they may integrate into neural structures after transplantation may allow for the regulated release of various gene products as well for literal cell replacement (While presently available gene transfer vectors usually depend on relaying new genetic information through established neural circuits, which may, in fact, have degenerated and require replacement, NSCs can participate in the reconstitution of these pathways.)

The replacement of enzymes and of cells may not only be targeted to specific anatomically circumscribed regions of CNS, but also to larger areas of the CNS by implantation into germinal zones. NSCs pass readily and unimpeded through the blood-brain barrier and deliver their foreign gene products immediately, directly, and in a disseminated fashion to the CNS. In addition, NSCs can be responsive to neurodegeneration, shifting the specifics of their differentiation to compensate for deficient cell types. The biology underlying these properties is not only of practical value, but also may illuminate some fundamental cell developmental mechanisms.

It will be noted and appreciated that the published patent and scientific literature provides an abundant basis of knowledge and information concerning human and other mammalian neural stem cells, their progeny progenitor cells, and their lineage-committed descendant cells. The identification and characterization of neural stem cells, obtained from a range of mammalian sources, are provided by U.S. Pat. Nos. 5,849,553; 5,928,947; 5,958,767; 5,968,829; and 6,103,530 respectively. A variety of methods for the isolation, purification, and maintenance in-vitro of neural stem cells and their offspring cells are described within U.S. Pat. Nos. 5,851,832; 5,750,376; 5,753,506; 5,958,767; 5,411,883; 5,082,670; 6,103,530; and 6,171,610. Particular techniques and processes for purposely inducing commitment to one cellular lineage and directing differentiation of descendant cells into becoming specific kinds of cells is provided by U.S. Pat. Nos. 5,980,885; 5,981,165; 6,001,654; 6,033,906; 6,071,889; 6,238,922; and 6,340,668. Each of these issued U.S. patents is expressly incorporated by reference herein.

### Lineage-Committed Cells and Partially-Differentiated Cells

NSCs are multipotent and self-renewing cells which can be repeatedly passaged in vitro and can become differentiated into numerous cell types of the body, including derivatives of the ectodermal and mesodermal tissues. NSCs are positive for nestin protein (an immunological marker of stem cells and progenitor cells) as well as for fibronectin protein; but are negative for vimentin or cytokeratin when assayed by immunohistochemistry. Moreover, NSCs can be grown as non-adherent clusters when cultured in-vitro; and one of ordinary skill in the art will recognize that such cells will grow as non-adherent clusters when cultured on a variety of substratum including but not limited to uncoated plastic or plastic coated with a neutral substrate such as gelatin or agar. NSCs are also negative for the neural crest stem cell marker p75. The differences in phenotype characteristics thus distinguish the NSCs from other types of adult stem cells, including hematopoietic stem cells, mesenchymal stem cells and neural crest stem cells.

Under certain conditions, the lineage-committed descendants of NSCs are capable of differentiating as dopaminergic neurons, and thus are a useful source of dopaminergic neurons. The descendant offspring cells of NSCs have also been demonstrated to make some mesodermal derivative cell types, including smooth muscle cells, adipocytes and bone. NSCs are believed to be capable of producing other mesodermal and endodermal types of differentiated cells, including cardiac muscle cells, pancreatic islet cells (e.g., alpha, beta, phi, delta cells), hematopoietic cells, hepatocytes, and the like.

Similarly, the lineage-committed cells of a NSC can fully differentiate as a neuron, an astrocyte, an oligodendrocyte, a Schwann cell, or a non-neural cell. Some differentiated forms of neurons include those neurons expressing one or more neurotransmitters such as dopamine, GABA, glycine, acetylcholine, glutamate, and serotonin. In comparison, some differentiated forms of non-neural cells exist as cardiac muscle cells, pancreatic cells (e.g., islet cells (alpha, beta, phi and delta cells)), exocine cells, endocrine cells, chondrocytes, osteocytes, skeletal muscle cells, smooth muscle cells, hepatocytes, hematopoietic cells, and adipocyes.

When completely differentiated, most of the descendant offspring cells lose their nestin positive immunoreactivity. In particular, antibodies specific for various neuronal or glial proteins may be employed to identify the phenotypic properties of completely differentiated cells. Neurons may be identified using antibodies to neuron specific enolase (“NSE”), neurofilament, tau, b-tubulin, or other known neuronal markers. Astrocytes may be identified using antibodies to glial fibrillary acidic protein (“GFAP”), or other known astrocytic markers. Oligodendrocytes may be identified using antibodies to galactocerebroside, O4, myelin basic protein (“MBP”) or other known oligodendrocytic markers.

It is also possible to identify cell phenotypes by identifying compounds characteristically produced by those phenotypes. For example, it is possible to identify neurons by the production of neurotransmitters such as acetylcyholine, dopamine, epinephrine, norepinephrine, and the like.

Specific neuronal phenotypes can be identified according to the specific products produced by those neurons. For example, GABA-ergin neurons may be identified by their production of glutamic acid decarboxylase (“GAD”) or GABA. Dopaminergic neurons may be identified by their production of dopa decarboxylase (“DDC”), dopamine or tyrosine hydroxylase (“TH”). Cholinergic neurons may be identified by their production of choline acetyltransferase (“ChAT”). Hippocampal neurons may be identified by staining with NeuN. It will be appreciated that any suitable known marker for identifying specific neuronal phenotypes may be used.

### Genetically Modified Cells

In addition, the human or other mammalian neural stem cells described herein can be genetically engineered or modified according to known methodologies. Any gene of interest (i.e., a gene that encodes a biologically active molecule) can be inserted into a cloning site of a suitable expression vector by using standard techniques. These techniques are well known to those skilled in the art. See for example, WO 94/16718, the text of which is expressly incorporated by reference herein.

The expression vector containing the gene of interest may then be used to transfect the desired cell line. Standard transfection techniques such as calcium phosphate co-precipitation, DEAE-dextran transfection, electroporation, biolistics, or viral transfection may be utilized. Commercially available mammalian transfection kits may be purchased from e.g., Stratagene. Human adenoviral transfection may be accomplished as described in Berg et al., Exp. Cell Res. 192: (1991). Similarly, lipofectomine-based transfection may be accomplished as described in Cattaneo, Mol. Brain Res. 42: 161-66 (1996).

A wide variety of host/expression vector combinations may be used to express a gene encoding a biologically active molecule of interest. See U.S. Pat. No. 5,545,723, herein incorporated by reference, for suitable cell-based production expression vectors.

Increased expression of the biologically active molecule can be achieved by increasing or amplifying the transgene copy number using amplification methods well known in the art. Such amplification methods include, e.g., DHFR amplification (see, U.S. Pat. No. 4,470,461) or glutamine synthetase (“GS”) amplification (see U.S. Pat. No. 5,122,464 and European published application EP 338,841, all of which are incorporated herein by reference).

The neural stem cells described herein, and their differentiated progeny may also be immortalized or conditionally immortalized using known techniques, preferably conditional immortalization of stem cells and most preferably conditional immortalization of their differentiated progeny. Among the conditional immortalization techniques contemplated are Tet-conditional immortalization (see WO 96/31242, incorporated herein by reference), and Mx-1 conditional immortalization (see WO 96/02646, incorporated herein by reference).

### Adult Stem Cell Category 4: Neural Crest Stem Cells and Their Offspring; Their Progenitor Cells, Their Lineage-Committed Cells and Their Partially-Differentiated Cells

Neural crest stem cells originate within and constitute part of the neural crest tissue in vertebrates. In turn, the neural crest of the vertebrate embryo serves as the main source of the cells forming the peripheral nervous system (PNS) and the autonomic nervous system (ANS). The neural crest itself is a transitory embryonic structure arising from the lateral ridges of the neural primordium autonomic nervous system; and develops into multiple lineages, including the sympathoadrenal lineage.

The sympathoadrenal lineage, being derived from the neural crest, gives rise primarily to chromaffin cells of the adrenal gland and sympathetic neurons. These two cell types can be distinguished at a number of levels. Chromaffin cells are small cells, without significant processes, and they secrete primarily the catecholamine epinephrine into the circulation. Sympathetic neurons are much larger cells, with dendritic and axonal processes that receive and send synaptic connections, respectively. These neurons secrete primarily norepinephrine, a catecholamine compound. While both cell types store their neurotransmitters and neuropeptides in vesicles, the chromaffin vesicles are about 150-350 nm in diameter, while neuronal synaptic vesicles are only about 50 nm in diameter. A third, apparently minor, population of cells in this lineage, known as small intensely fluorescent (SIF) cells, is somewhat intermediate in character between neurons and chromaffin cells and has vesicles of 75-120 nm in diameter.

In addition to these chemical and morphological characteristics, chromaffin cells and sympathetic neurons can be distinguished by numerous molecular markers, such as genes encoding specific cytoskeletal, vesicle and surface proteins; and neurotransmitter-synthesizing enzymes such as phenyl-N-methyl transferase (which is chromaffin cell specific). These markers have proven useful both in the identification of intermediate stages of differentiation and in the identification and isolation of a sympathoadrenal progenitor cell.

The step at which a multipotent neural crest cell becomes committed to the sympathoadrenal lineage is not well understood. Catecholamine-producing cells that resemble neurons or chromaffin cells have been observed in cultures of neural crest cells but these possible sympathoadrenal precursors have not as yet been isolated for further study. Committed sympathoadrenal progenitors that are at least bipotential (giving rise to either chromaffin cells or sympathetic neurons) have, however, been isolated from embryonic adrenal medullae as well as embryonic and neonatal rat sympathetic ganglia. Embryonic progenitors can be isolated by fluorescence-activated cell sorting (FACS), using several surface membrane antigens. In cell culture, these progenitors can be induced to become neurons or chromaffin cells that express cell type-specific antigens or genes, as well as the appropriate morphology and ultra-structure.

For more detailed information, empirical evidence, and reviews of the sympathoadrenal lineage system, the reader is directed to the following list of relevant and representative publications: Patterson et al., Cell 62: 1035-1038 (1990); Stemple, D. L. and D. J. Anderson, Cell 71: 973-985 (1992); Anderson, D. J., Ann. Rev. Neurosci. 16: 129-158 (1993); Le Douarin, N. M., Science 231: 1515-1522 (1986); Kim et al., J. Neurosci. Res. 22: 50-59 (1989); Lo et al., Dev. Biol. 145: 139-153 (1991); Jessen et al., Neuron 12: 509-527 (1994); Anderson, D. L., Curr. Opin. Neurobiol. 3: 8-13 (1993); and the references cited within each of these publications.

The avian embryo has also provided considerable information via in-vivo and in-vitro experimental studies of the lineage patterning of neural crest stem cell descendants. For more detailed information, empirical data, and reviews of the avian lineage analyses for neural crest cells, the reader is directed to the following representative publications: Le Douarin et al., Dev. Biol. 159: 24-49 (1993); N. M. Le Douarin, Nature 286: 663-669 (1980); N. M. Le Douarin, The Neural Crest, Cambridge University Press, Cambridge, U.K., 1982; Sieber-Blum et al., Dev. Biol. 80: 96-106 (1980); Baroffio et al., Proc. Natl. Acad. Sci. USA 85: 5325-5329 (1988); Bronner-Fraser et al., Neuron 3: 755-766 (1989); Cohen et al., Dev. Biol. 46: 262-280 (1975); Dupin et al., Proc. Natl. Acad. Sci. USA 87: 1119-1123 (1990); Duff et al., Dev. Biol. 147: 451-459 (1991); Bronner-Frasier et al., Nature 355: 161-164 (1988).

### The Mammalian Neural Crest Stem Cell

The isolation and characterization of neural crest stem cells to date has been the consequence of the ongoing effort to seek out, isolate and portray a “stem cell” for neurons and glia which is derived from the mammalian neural crest. Most illustrative of this approach are the scientific publications and issued U.S. patents of D. J. Anderson and his colleagues identified below. In the main, these publications have identified mammalian neural crest cells—chiefly rat and mouse cells—using antibodies to cell surface antigens; and have used sub-cloning to examine the developmental potential of these cells and their progeny.

Three main findings emerge from their analyses. First, mammalian neural crest stem cells are pluripotent cells. Second, multipotent neural crest stem cells generate pluripotent progeny, indicating that these offspring are capable of self-renewal and therefore are also stem cells. Third, neural crest stem cells also generate some descendant offsrping cells that form only neurons or glia, showing that these stem cells eventually produce lineage-committed neuroblasts and glioblasts. The empirical data also shows that in-vivo neural crest stem cells exist in living mammals; maintain their pluriipotency as they continue to proliferate; and that lineage-committed pathway choices occur within developing ganglia via the generation of committed blast cells.

Among the relevant scientific publications which document and support these findings are: D. J. Anderson, Neuron 3: 1-12 (1989); D. J. Anderson, Annu. Rev. Neurosci. 16: 129-158 (1993); Stemple, D. L. and D. J. Anderson, Cell 71: 973-985 (1992); Anderson et al., J. Neurosci. 11: 3507-3519 (1991); Anderson, D. L. and R. Axel, Cell 42: 649-662 (1985); Anderson, D. L. and R. Axel, Cell 47: 1079-1090 (1986); Shah et al., Cell 85: 331-343 (1996); Lo et al., Dev. Biol. 145: 139-153 (1991).

The reader is also directed to several issued U.S. patents for further information and guidance. These are U.S. Pat. Nos. 6,001,654; 5,928,947; 5,849,553; 5,693,482; 5,824,489; 5,654,183; and 5,589,376. The texts of all these issued U.S. patents, individually and collectively, are expressly incorporated by reference herein.

The human neural crest stem cell, its isolation, maintenance in-vitro, and phenotopic characterization has been described in full by Seung U. Kim and his colleagues; and a stable clone of human neural crest stem cells has been prepared and characterized in depth. See for example: “Production Of Immortalized Human neural Crest Stem Cells” in Neural Stem Cells: Methods and Protocols, Humana Press, Totowa, N.J., 2002; and PCT Publication No. WO-.

To characterize the phenotopic range of human neural crest stem markers, a listing of the representative identifying antigens is provided by Table 2 below.

TABLE 2
Human Neural Crest Stem Cell Characteristics

| | Presence (+) | Meaning/Value | |
|---|---|---|---|
| Marker | or | of Marker | Substance |
| (Type) | Absence (−) | Indicator | Detected |
| human mitochondria⁽¹⁾ | (+) | human cell origin | cell organelle |
| pan-myc oncoprotein⁽²⁾ | (+) | contains vmyc | cellular oncoprotein |
| nestin⁽³⁾ | (+) | NCSC | cytoskeletal protein |
| p75NGFR⁽⁴⁾ | (+) | NCSC | surface receptor antigen |
| vimentin⁽⁵⁾ | (+) | NCSC | cytoskeletal protein |
| A2B5⁽⁶⁾ | (+) | NCSC | surface antigen |
| Musashi⁽⁷⁾ | (+) | neural progenitor | transcription |
| | | cells | factor |
| FORSE-1⁽⁸⁾ | (+) | neural progenitor | surface antigen |
| | | cells | |
| PSA-NCAM⁽⁹⁾ | (+) | neural progenitor | surface antigen |
| | | cells | |
| NG2 proteoglycan⁽¹⁰⁾ | (+) | progenitor cells/ | surface antigen |
| | | glial precursor cells | |
| NF-L^{(II)} | (+) | early stage neuron | specific neuron marker |
| NF-M^{(II)} | (+) | middle stage neuron | specific neuron marker |
| NF-H^{(II)} | (+) | differentiated neuron | specific neuron marker |
| β tubulin^{(II)} | (−) | differentiated neuron | specific neuron marker |
| isotype III | | | |
| peripherin^{(II)} | (−) | mature peripheral | PNS specific neuron |
| | | nervous system neuron | marker |
| trkA^{(II)} | (−) | neuron | neurotrophin (NGF) |
| | | | receptor protein |
| O1^{(II)} | (−) | oligodendrocyte | specific oligodendrocyte |
| | | | marker |
| O4^{(II)} | (−) | oligodendrocyte | specific oligodendrocyte |
| | | | marker |
| GFAP^{(II)} | (−) | astrocyte cell | specific astrocyte |
| | | | marker |
| MBP^{(II)} | (−) | oligodendrocyte | specific oligodendrite |
| | | | marker |
| B7-2^{(II)} | (−) | microglia | specific marker |
| PO^{(II)} | (−) | Schwann cell | specific Schwann |
| | | | cell marker |
| trkB/trkC^{(II)} | (−) | special classes of | neurotrophin receptor |
| | | neurons | protein |
| S100^{(III)} | (−) | glial cell | mature Schwann |
| | | | cell marker |
| chromogranin^{(IV)} | (−) | adrenal | chromaffin cell |
| | | chromaffin cell | specific marker |
| desmin^{(V)} | (−) | skeletal muscle | cytoskeletal protein |
| | | cell | |
| myosin^{(V)} | (−) | skeletal muscle | cytoskeletal protein |
| | | cell | |

Table 2 References:
⁽¹⁾Flax et al., Nature Biotech 16: 1033-1039 (1998).
⁽²⁾Spotts, R. and B. Hann, Mol. Cell Biol. 10: 3952-3956 (1990).
⁽³⁾Lendahl et al., Cell 60: 585-595 (1990); Stemple, D. L. and D. J. Anderson, Cell 71: 973-985 (1992); Friedman et al., J. Comp. Neurol. 295: 43-51 (1990); Hockfield et al., J. Neurosci. 5: 3310-3328 (1985); Reynolds et al., Science 255: 1707-1710 (1992).
⁽⁴⁾Chao, M. V., Neuron 9: 583-593 (1992); Morrison et al., Cell 96: 737-749 (1999).
⁽⁵⁾Houle, J. and S. Fedoroff, Dev. Brain Res. 9: 189-195 (1983).
⁽⁶⁾Eisenbarth et al., Proc. Nat. Acad. Sci. USA 46: 4913-4917 (1979).
⁽⁷⁾Sakakibara, S. and H. Okano, J. Neurosci. 17: 8300-8312 (1997).
⁽⁸⁾Tole, S. and P. H. Patterson J. Neurosci. 15: 970-980 (1995).
⁽⁹⁾Grinspan, J. B. and B. Franceschino, J. Neurosci. Res. 41: 540-551 (1995).
⁽¹⁰⁾Stallcup, W. B. and L. Beasley, J. Neurosci. 7: 2737-2744 (1987).
^{(II)}See Table 3 references.
^{(III)}See Table 4 references.
^{(IV)}See Table 5 references.
^{(V)}See Table 6 references.

### Lineage-Committed Cells and Partially-Differentiated Cells

Human neural crest cells and their progenitor progeny cells can commit to at least four different pathway lineages when cultured in-vitro; which, in turn, will result in and yeild a variety of partially-differentiated cells and then terminally differentiated cells. These pathway lineages are: the neuronal cell line; the glial cell line; the adrenal chromaffin cell line; and the skeletal muscle cell line.

A variety of different immunological and histochemical markers have been identified in the published scientific literature which offers reliable surface antigen phenotypes by which to separate and distinguish the different possible choices of cell lineage development. The range of identifying markers for a specific cell lineage pathway are described by Tables 3-6 respectively below.

TABLE 3
Neuronal Cell Lineage Characteristics

| | Absence (−) | Meaning or | |
|---|---|---|---|
| Marker | or | Value of | Substance |
| (Type) | Presence (+) | of Indicator | Detected |
| NF-L⁽¹⁾ | (+) | early stage neuron | neurofilament protein- |
| | | | specific neuron marker |
| NF-M | (+) | middle stage | neurofilament protein- |
| | | neuron | specific neuron marker |
| NF-H | (+) | mature neuron | neurofilament protein- |
| | | | specific neuron marker |
| β tubulin⁽²⁾ | (+) | mature neuron | specific neuron marker |
| isotype III | | | |
| peripherin⁽³⁾ | (+) | mature peripheral | specific neuron marker |
| | | nervous system | |
| | | neuron | |
| trkA⁽⁴⁾ | (+) | sensory and | receptor protein specific |
| | | sympathetic | for NGF |
| | | neurons | |
| trkB/trkC⁽⁵⁾ | (−) | special classes of | receptor protein specific |
| | | neurons | for BDNF (trkB) or |
| | | | NT-3 (trkC) |

Table 3 References:
⁽¹⁾Julien et al., Biochem. Biophys. Acta. 909: 10-20 (1987); Lee et al., EMBO J. 7: 1947-1955 (1988); Myers et al., EMBO J. 6: 1617-1626 (1987).
⁽²⁾Ferreira, A. and A. Caceres, J. Neurosci. Res. 32: 516-529 (1992).
⁽³⁾Parysek, L. M. and R. D. Goldman, J. Neurosci. 8: 555-563 (1988); Gorham et al., Dev. Brain Res. 57: 235-248 (1990).
⁽⁴⁾Chao, M. V., Neuron 9: 583-587 (1992).
⁽⁵⁾Obermeyer et al., J. Biol. Chem. 268: 22963-67 (1993).

TABLE 4
Glial Cell Lineage Characteristics

| | Absence (−) | Meaning or | |
|---|---|---|---|
| Marker | or | Value of | Substance |
| (Type) | Presence (+) | Indicator | Detected |
| glial fibrillary⁽¹⁾ | (+) | astrocyte and | cellular protein of |
| acidic protein | | Schwann cell | astrocyte and |
| (GFAP) | | | Schwann cells |
| S100⁽²⁾ | (+) | Schwann cells | Schwann cell |
| | | | specific marker |
| O1⁽³⁾ | (−) | oligodendrocyte | oligodendrocyte |
| | | | specific marker |
| O4⁽⁴⁾ | (−) | oligodendrocyte | oligodendrocyte |
| | | | specific marker |
| MBP⁽⁵⁾ | (−) | oligodendrocyte | oligodendrocyte |
| | | | specific marker |
| PO⁽⁶⁾ | (+) | Schwann cell | Schwann cell |
| | | | specific marker |
| B7-2⁽⁷⁾ | (−) | microglia | microglia specific |
| | | | marker |

Table 4 References
⁽¹⁾Jessen et al., Neuron 12: 509-527 (1994).
⁽²⁾Jessen et al., Development 109: 91-103 (1990).
⁽³⁾Sommer, L. and M. Schachner, Dev. Biol. 83: 311-327 (1981).
⁽⁴⁾Schachner et al., Dev. Biol. 83: 328-338 (1981).
⁽⁵⁾Sternberger et al., Proc. Nat. Acad. Sci. USA 75: 2521-2524 (1978).
⁽⁶⁾Jessen et al., Neuron 12: 509-527 (1994).
⁽⁷⁾Satoh et al., Brain Res. 704: 92-96 (1995).

TABLE 5
Lineage Characteristics of Adrenal Chromaffin Cells

| | Absence (−) | Meaning or | |
|---|---|---|---|
| Marker | or | Value of | Substance |
| (Type) | Presence (−) | Indicator | Detected |
| chromogranin⁽¹⁾ | (+) | adrenal chromaffin | cell surface |
| | | cell | marker |
| tyrosine hydroxylase⁽²⁾ | (+) | sympathetic neurons | specific |
| | | of the autonomic | enzyme |
| | | nervous system | |
| PNMT⁽³⁾ | | adrenal chromaffin | cell marker |
| | | cell | |

Table 5 References
⁽¹⁾Wilson, B. S. and R. V. Lloyd, Am. J. Pathol. 115: 458-468 (1984).
⁽²⁾Pickel et al., Proc. Nat. Acad. Sci. USA 72: 659-663 (1975).
⁽³⁾Connett, R. and N. Kirschner, J. Biol. Chem. 245: 329-334 (1970).

TABLE 6
Lineage Characteristics of Skeletal Muscle Cells

| | Absence (+) | Meaning or | |
|---|---|---|---|
| Marker | or | Value of | Substance |
| (Type) | Presence (−) | Indicator | Detected |
| morphologically flat, | (+) | myoblasts | physical |
| elongated, multinucleated | | | appearance |
| cells⁽¹⁾ | | | of cells |
| desmin⁽²⁾ | (+) | skeletal | cellular |
| | | muscle cell | filament |
| myosin⁽³⁾ | (+) | skeletal | cellular |
| | | muscle cell | filament |

Table 6 References
⁽¹⁾Le Douarin et al., Dev. Biol. 159: 24-49 (1993).
⁽²⁾Naumann, K. and D. Pette, Differentiation 55: 203-211 (1994).
⁽³⁾Ibid.

### V. Experiments and Empirical Data

To demonstrate the merits and value of the present invention, a series of planned experiments and empirical data are presented below. It will be expressly understood, however, that the experiments described and the results provided are merely the best evidence of the subject matter as a whole which is the invention; and that the empirical data, while limited in content and design, is only illustrative and representative of the scope of the invention envisioned and claimed.

### Experimental Series I

Despite considerable advances in biomedical sciences, the incidence of heart failure remains high in patients after myocardial infarction (MI). This experimental study investigated the effects of intramyocardial transplantation of embryonic stem cells (ESCs) with or without tansfection of a cDNA of vascular endothelial growth factor (phVEGF₁₆₅) on infarcted heart function.

### Background

After myocardial infarction (MI), dead myocardium is replaced by noncontractile fibrous scar which leads to ventricular dysfunction. Significant advances have been made in diagnosis and treatment of heart diseases in the last several decades, but effective therapy for heart failure remains a great challenge for clinicians. The morbidity and mortality of heart failure are significantly higher in developed countries. Limited proliferation of endogenous myocardial cells in infarcted myocardium has been reported, but the massive loss of mammalian cardiomyocytes due to ischemia is not regenerated by the remaining myocytes.

Cardiomyogenic cells derived from embryonic stem cells (ESCs) can be a viable source for donor cardiomyocytes. ESCs derived from the inner cell mass of the mouse preimplantation blastocyst are pluripotent cells and retain the ability to differentiate in vitro into numerous cell types, including spontaneously contracting cardiomyocytes. In addition, differentiation of ESCs to cardiomyogenic cells is accompanied by the expression of a number of cardiac and muscle-specific contractile proteins, including cardiac α- and β-myosin heavy chain, α-tropomyosin, phospholamban and type B natriuretic factor. It has also been shown that transplantation of cultured cardiomyocytes from differentiating murine ESCs formed stable intracardiac grafts. Thus, transplantation of ESCs might yield new cardiomyocytes to repair injured myocardium and improve cardiac function.

One of the most important growth and survival factors for endothelium is vascular endothelial growth factor (“VEGF”). VEGF induces angiogenesis and endothelial cell proliferation, and plays an important role in regulating angiogenesis. VEGF is a heparin-binding glycoprotein that is secreted as a homodimer of 45 kDa. The development of new blood vessels, or angiogenesis, begins with activation of parent vessel endothelial cells. In the case of a major artery obstruction, blood flow to the ischemic tissue is often dependent on collateral vessels. It has been demonstrated in vivo and in patients that VEGF improves collateral blood flow in ischemic regions; and, more recently, that direct injection of phVEGF₁₆₅ alone improved myocardial blood perfusion in patients with myocardial ischemia.

This Experimental Series I was therefore designed to evaluate whether transplanted ESCs could survive in injured myocardium and improve cardiac function in MI mice. In addition, the experiments determined whether transplantation of ESCs with overexpression of VEGF produced a greater effect on improvement of cardiac function in MI mice. The effects of transplantation of ESCs alone or ESCs with overexpression of VEGF on neovascularization in ischemic myocardium were also evaluated and compared.

### Materials and Methods

### Culture of Embryonic Stem Cells and Progenitor Progeny Cells

The mouse cell line, ES-D3, was obtained from the American Type Culture Collection (ATCC, Manassas, Va.) and maintained in DMEM (Gibco BRL, Grand Island, N.Y.) on mitotically inactive mouse embryonic fibroblast feeder cells (ATCC). The medium was supplemented with 15% fetal bovine serum (FBS), 0.1 mM β-mercaptoethanol, and 10³ Units/ml of leukemia inhibitory factor (LWF) (Gibco BRL, Grand Island, N.Y.). To initiate differentiation, ESCs and their progenitor progeny cells were dispersed with trypsin and resuspended in the medium without supplemental LIF; and cultured with the hanging drops (approximate 400 cells per 20 μl) method for two days using the technique of Wobus et al. [Differentiation 48: 177-182 (1991)]. The resulting embryoid bodies were transferred from the hanging drops into 100 mm-dishes and cultured for another 5 days. Beating cardiomyogenic clusters, the lineage-committed but undifferentiated descendant offspring, were dissected by use of a sterile micropipette [Maltser et al., Mech. Dev. 191: 42-50 (1993)]; and cultivated the cells into 100 mm culture dishes. Action potentials were recorded in cultured cardiac-like stem cells by the current-clamp technique [Xiao, Y. F. and J. P. Morgan, J. Pharmacol. Exp. Ther. 284: 10-18 (1998)] and cell-shortening was measured by the edge-detected method [Xiao et al., J. Physiol. (Lond) 508: 777-792 (1998)].

### Transfection of Green Fluorescent Protein Gene

Before cell transplantation, ESCs were transfected with a cDNA of green fluorescent protein (GFP) to identify the survival of implanted cells. Plasmid with hCMVIE promoter/enhancer driving green fluorescent protein gene (5.7 kb) and GenePORTER™ transfection reagent was obtained from Gene Therapy System, Inc. (GTS Inc., San Diego, Calif.). Briefly, ESCs were plated in 100-mm dishes and cultured to 60% confluent on the day of transfection. The plasmid GFP DNA (8 μg) was added to each dish with a calcium phosphate precipitation method [Xiao et al., Am. J. Physiol. 279: H35-H46 (2000)]. After 2 days of GFP transfection, cultured ESCs were trypsinized and resuspended in Joklik modified medium (Sigma, St. Louis, Mo.) with a density of 10⁷ cells/ml. Stable GFP expression was detected in cultured cells for 8 weeks.

### Transfection of Plasmid VEGF DNA (phVEGF₁₆₅)

The plasmid of VEGF cDNA (phVEGF₁₆₅) was a generous gift from Dr. Kenneth Walsh (St. Elizabeth's Medical Center, Tufts University School of Medicine, Boston, Mass.). It is a eukaryotic expression plasmid that uses the 736 bp CMV promoter/enhancer to drive VEGF expression [Leung et al., Science 244: 1306-1309 (1989)]. 60-80% confluent ESCs were transfected with 8 μg phVEGF₁₆₅ per 100-mm dish according to the manufacture's protocol (Gibco BRL, Grand Island, N.Y.). Cells were trypsinized 48 hours post transfection and were resuspended in Joklik modified medium for transplantation. Overexpression of VEGF in cultured ESCs was observed by immunoflurescent assay. In brief, after 48 hours transfection of phVEGF₁₆₅, ESCs were washed with phosphate-buffered saline (PBS) twice and then fixed in 4% paraformaldehyde. A rabbit anti-human VEGF antibody (Santa Cruz Biotechnology Inc, Santa Cruz, Calif.) was used to incubate with ESCs. A goat anti-rabbit IgG conjugated fluorescein (Pierce Chemical Company, Rockford, Ill.) was as a second antibody to test fluorescence. Western blot analysis of VEGF with with Leung et al. method also showed a significant increase in VEGF-transfected ESCs.

### Animal Model of MI and ESCs Transplantation

The experiments were performed on male FVB mice (Charles River, Wilmington, Mass.) 8-12 wk of age (24-35 g body weight). Myocardial infarction was induced by ligation of the left anterior descending coronary artery as described previously [Michael et al., Am. J. Physiol. 269: H2147-H2154 (1995)]. Briefly, animals were anesthetized by intraperitoneal injection of pentobarbital sodium, 40 μg body weight. A midline cervical skin incision was made and the endotracheal tube was placed in the trachea. A lateral incision between fourth and fifth ribs was made to open the chest. A rodent ventilator (Harvard Apparatus Inc, Holliston, Mass.) was connected to the endotracheal tube to maintain animal respiration after opening the chest. The heart was oriented to better expose the left main coronary artery system. Ligation proceeded with a 6-0 silk suture passed with a tapered needle underneath the left anterior descending branch of the left coronary artery, 2 mm down of the tip of the normally positioned left auricle. ESCs suspension (3×10⁵ in 30 μl) was separately injected into ischemic myocardium 10 to 15 minutes after MI induction. Another MI group was transplanted with the same amount of cells with overexpression of VEGF. Control MI animals received the same MI operation, but were only injected with the equivalent volume of the cell-free medium. The sham group underwent the identical surgery with neither ligation of the coronary artery nor cell transplantation. The experimental protocol was approved by the Animal Care Committee of Beth Israel Deaconess Medical Center and performed according to the Guide for the Care and Use of Laboratory Animals published by the US National Institutes of Health (NIH Publication No.85-23, revised 1996).

### Measurements of Hemodynamics and Isometric Contraction

Six weeks after MI operation and cells transplantation, hemodynamic measurements in vivo were performed with the methods described previously [Gao et al., Cardiovasc. Res. 45: 330-338 (2000)]. After the measurements, the heart was rapidly removed from the sacrificed mouse. Left ventricular papillary muscle strips were dissected and vertically connected to a train-gauge tension transducer. Developed tension (DT) of muscle strips was recorded at their maximal length. The bath solution contained the modified Krebs-Henseleit solution and different concentrations of isoproterenol (10⁻⁶, 10⁻⁵, and 10⁻⁴ M). The inotropic response of dissected ventricular papillary muscles from MI hearts to β-adrenergic stimulation was evaluated in MI control and cell-transplanted MI mice.

### Histological and Immunofluorescent Analysis

The subsets of animals were sacrificed after 6 weeks of MI induction. After quickly removing the hearts, the free wall of the left ventricle including the infarcted and peri-infarcted regions were embedded in tissue freezing medium (Fisher Scientific, Fair Lawn, N.J.). Frozen tissue was sectioned to 10-μm slides and stained with hematoxylin and eosin (HE). Survival of engrafted cells was confirmed by identification of GFP-positive spots under fluorescent microscopy.

To identify regenerated myocytes from engrafted ESCs, we used an immunofluorescent technique to detect cardiac troponin-I (cTn-I) and a-myosin heavy chain (a-MHC), two of the structural markers of myocardium. Frozen tissue sections were fixed in acetone for 10 minutes and then dried in air. Nonspecific binding was blocked by incubation in 1% bovine serum albumin in PBS. The samples were then reacted with an anti-troponin-I antibody (goat polyclonal IgG, Santa Cruz Biotechnology Inc, Santa Cruz, Calif.), or a mouse anti-α-MHC monoclonal antibody (Berkeley Antibody Company, Richmond, Calif.) for 1 hour. After washing with PBS, sections were incubated with a rabbit anti-goat conjugated rhodamine IgG (H+L) for cTn-I or a goat anti-mouse conjugated fluorescein IgG for α-MHC (Pierce Chemical Company, Rockford, Ill.). Fluorescent immunostaining for cTn-I and α-MHC was examined and photographed under fluorescent microscopy.

Double staining for GFP (ZYMED Lab, Inc, San Francisco, Calif.) and connexin43 (CX-43, Sigma, St. Louis, Mo.) was carried out with the mouse anti-GFP and rabbit anti-CX-43 antibodies in myocardial frozen section to verify the formation of gap junction in cell-transplanted myocardium. GFP labeling was detected with a goat anti-mouse antibody conjugated to FITC (Pierce Chemical Company, Rockford, Ill.). CX-43 labeling was detected with a goat anti-rabbit antibody conjugated to Texas Red (Vector Lab, Inc, Burlingame, Calif.). Confocal microscopy was applied to analyze immunofluorescence labeling of engrafted cells in injured myocardium with the antibodies recognizing GFP and CX-43.

### Assessment of Angiogenesis

The density of capillary vessels was examined in injured-HE staining myocardium with or without ESCs transplantation. The number of capillaries, less than 20 μm, was counted in the ischemic zone of all groups. Each section was randomly detected in five high-power fields (×400 magnification) and the number of capillaries in each microscopy field was presented as the mean of blood vessels per unit area (0.2 mm²). In addition, we also used immunohistochemical staining for blood vessel endothelial cells by anti-von Willebrand Factor (vWF, DAKO LSAB Kit, DAKO Corp, Carpinteria, Calif.) antibody to evaluate ESCs-induced angiogenesis in infarcted myocardium. After tissue fixation in acetone, frozen sections were treated with 3% hydrogen peroxide for 5 minutes and then incubated with a rabbit anti-human vWF IgG. Following PBS washing, the sections were linked with a biotinylated link antibody and labeled with streptavidin. A mixed substrate-chromogen solution was used to incubate with sections. Finally, sections were stained with hematoxylin for 2 minutes. Images were captured with the Spot Software (Version 2. 1, Diagnostic Instruments Inc, Sterling Heights, Mich.).

### Data Analysis

The averaged data are expressed as mean ±SE. Statistical significance between two groups was determined by paired or unpaired Student's t-test. Results for more than two experimental groups were evaluated by one way variance analysis (ANOV A) with repeated measurements to specify differences between groups. A P value less than 0.05 was considered as significant difference.

### Empirical Results

### Experiment 1: Differentiation of ESCs

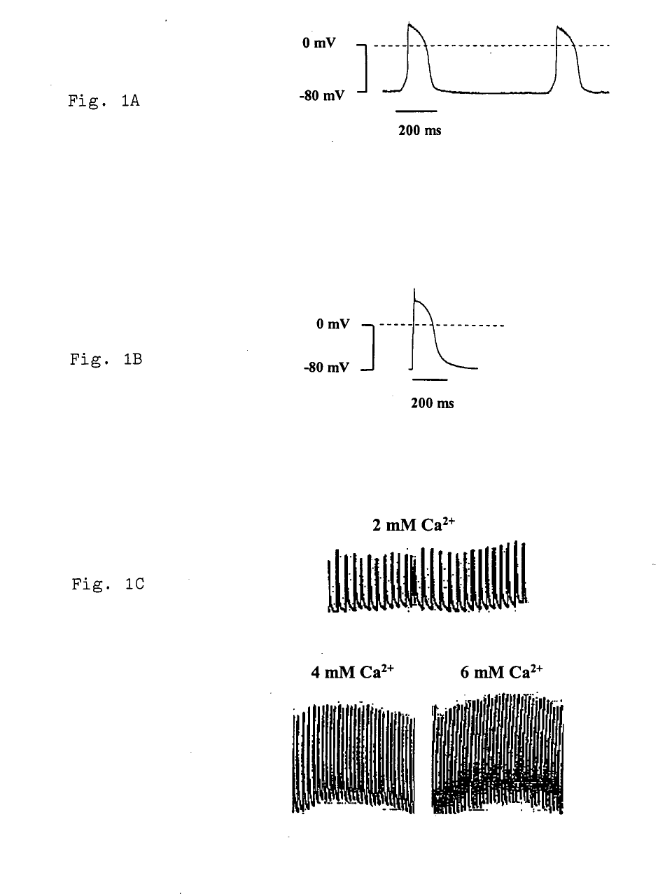

Spontaneously beating cells were observed in cultured ESCs after 9 to 14 days of withdrawal of LIF from conditioned culture medium. The action potentials recorded from ESCs-derived cardiomyocytes are shown by FIGS. 1A-1C respectively.

ESCs were cultured for ˜10 days after withdrawal of LIF from cultured medium. Spontaneous action potentials were observed with the zero-current clamp method in a representative beating cell, as shown by FIG. 1A. Electrical-stimulated action potential was elicited in an ESC cell after depolarizing the membrane potential by the current clamp method, as shown by FIG. 1B. Finally, FIG. 1C shows the response of cell shortening and spontaneously beating to changes to changes of extracellular Ca²⁺ concentration in cultured ESCs by the edge-detected method.

Thus, the data shows that some of the beating cells had spontaneous action potentials recorded by the zero current-clamp technique (FIG. 1A); while some of the others had no spontaneous action potential, but were elicited action potentials after electrical stimulation (FIG. 1B). Also the data shows that increase in extracellular Ca²⁺ concentration enhanced the rate of spontaneous contractions and the amplitude of cell shortening in differentiated cells from cultured mouse ESCs (FIG. 1C). These results demonstrate that after ˜10 days in culture, ESCs in the absence of LIF are able, at least part of them, to differentiate to spontaneously beating cardiac-like cells.

### Experiment 2: Improvement of Myocardial Contractility after ESCs Transplantation

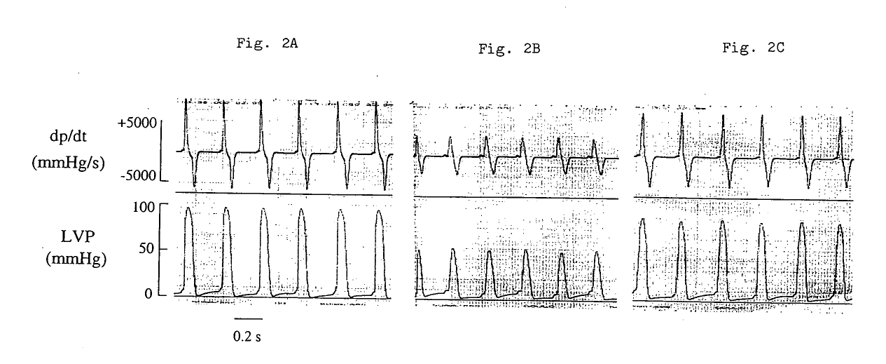

Six weeks after MI induction, hemodynamic measurements showed that the MI mice injected with the cell-free medium had a lower LV systolic pressure (LVSP, P<0.01), a higher LV end-diastolic pressure (LVEDP, P<0.01), and a slower rate to reach the peak of LV systolic pressure (+dP/dt, P<0.01) than those in sham operated mice (see Table E1). However, ESCs implantation significantly improved the left ventricular function at 6 weeks after MI induction and cell transplantation. Myocardial contractility reflected by the parameters of LVSP, LVEDP, and +dP/dt was significantly increased in cell-transplanted mice (P<0.05, versus MI control). This is shown by FIGS. 2A-2C.

FIG. 2 shows the original traces of hemodynamic measurements in MI mice. FIG. 2A represents the sham-operated control; FIG. 2B represents the MI control with injection of the cell-free medium; and FIG. 2C represents the MI with intramyocardial transplantation of ESCs. Measurements were conducted after 6 weeks of MI induction and cell transplantation. LVP, left ventricular pressure; dP/dt, rate of left ventricular pressure change.

TABLE E1
Improvement of left ventricular function
by ESCs transplantation in MI mice

| | Sham | MI + Medium | MI + ESCs |
|---|---|---|---|
| | (n = 8) | (n = 7) | (n = 8) |
| LVSP (mmHg) | 94.2 ± 2.3 | 50.5 ± 2.0** | 79.8 ± 2.1^{#} |
| LVEDP (mmHg) | 9.6 ± 0.3 | 18.2 ± 0.7** | 12.5 ± 0.4_{#} |
| +dP/dt (mmHg/s) | 8100 ± 220 | 4600 ± 310** | 7200 ± 260^{#} |

Values are presented as mean ±+SE.
Sham: sham-operated mice;
MI + Medium: myocardial infarction mice injected with cell-free medium;
MI + ESCs: myocardial infarction mice transplanted with ESCs.
LVSP: left ventricular systolic pressure;
LVEDP: left ventricular end-diastolic pressure;
+dP/dt: rate of peak left ventricular systolic pressure raise.
**P < 0.01 vs. Sham;
^{#}P < 0.05 vs. MI + Medium.

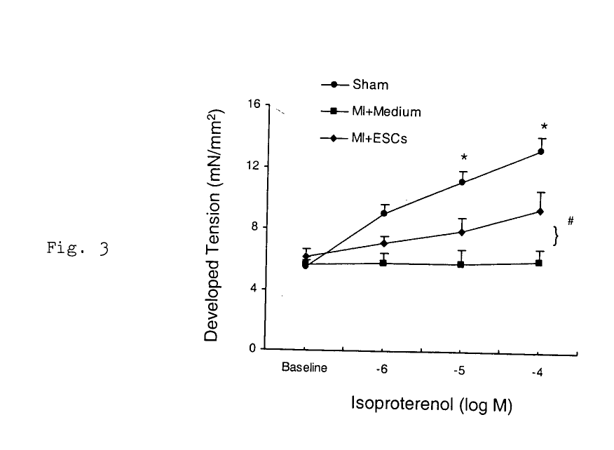

The empirical data also showed the inotropic response to the β-adrenergic stimulation in isolated papillary muscles. This is illustrated by FIG. 3. An increase in developed tension to isoproterenol stimulation was observed in papillary muscles isolated from sham-operated mice (Sham, , n=8). However, the positive inotropic response to isoproterenol stimulated was blunted in MI+Medium group ( , n=7). In contrast, developed tension to the β-adrenergic stimulation was significantly preserved in papillary muscles isolated from post-infarcted mice transplanted with ESCs (MI+ESCs, , n=8). Data are expressed as mean ±SE. *, P<0.05, vs. Sham; #, P<0.05, vs. MI+Medium.

These results demonstrated that the developed tension of isolated papillary muscles was similar at the baseline for the sham-operated mice and for the MI mice with or without cell transplantation (FIG. 3, see Baseline). Beta-adrenergic stimulation with different concentrations of isoproterenol significantly increased the developed tension of papillary muscles isolated from the sham-operated mice (FIG. 3, Sham). In contrast, papillary muscles isolated from the MI mice with intramyocardial injection of the cell-free medium did not response to the β-adrenergic stimulation well (FIG. 3, MI+Medium). However, papillary muscles isolated from MI mice transplanted with ESCs remarkably responded to the stimulation of isoproterenol (FIG. 3, MI+ESCs), especially at 10⁻⁴ M of the β-adrenergic agonist (P<0.05, versus MI+Medium). This result indicates that intramyocardial transplantation of ESCs partially preserved the contractility of the left ventricular papillary muscles.

### Experiment 3: Histologic Analysis

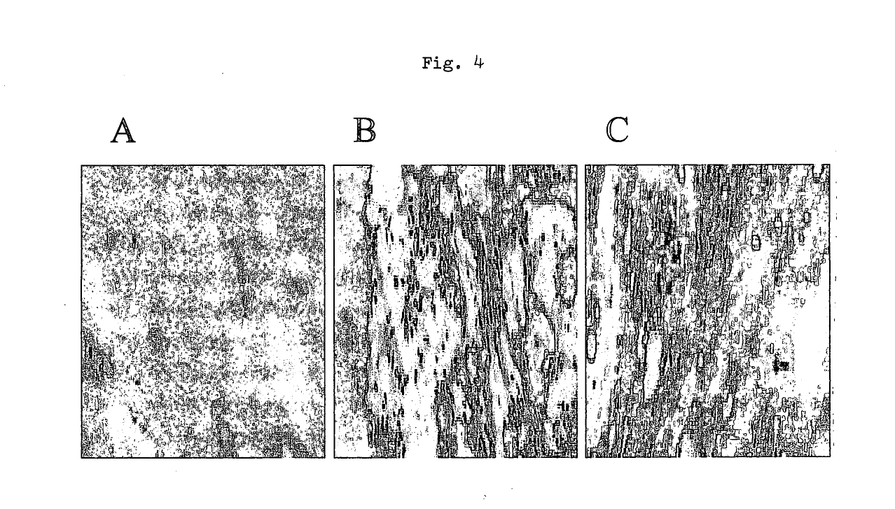

An analysis was done to show morphological changes after MI induction and cell transplantation. The results are shown by FIGS. 4A-4C respectively.

FIG. 4A illustrates HE staining in sham-operated mouse heart tissue (Sham); FIG. 4B shows the MI mouse injected with cell-free medium (MI+Medium); and FIG. 4C represents the MI mouse injected with ESCs (MI+ESCs). Original magnification ×200.

Thus, the empirical evidence shows that normal myocardium is observed in the sham-operated mice by hematoxylin and eosin staining (FIG. 4A). However, necrotic and fibrous scar tissue was clearly formed in the MI myocardium injected with the cell-free medium after 6 weeks of ligation of the left anterior descending coronary artery (FIG. 4B). In contrast, engrafted cells were found in infarcted hearts 6 weeks after MI induction and cell transplantation (FIG. 4C). This result reveals that engrafted ESCs survive in injured myocardium.

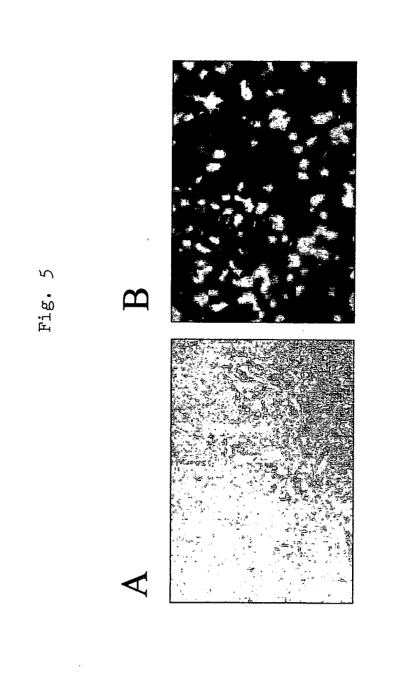

To further confirm the survival of implanted cells, the marker GFP cDNA was transfected into cultured ESCs before they were transplanted into myocardium. The results are illustrated by FIGS. 5A-5C.

FIG. 5 demonstrates GFP expression in cultured ESCs and in myocardium with cell transplantation. FIG. 5A shows cultured ESCs under light-contrast microscopy (×200); FIG. 5B illustrates GFP expression in cultured ESCs 2 days after GFP transfection under fluorescent microscopy (×200); and FIG. 5c identifies GFP positive spots detected under fluorescent microscopy (×100) in myocardium sectioned from a MI heart with transplantation of GFP-transfected ESCs.

This histological analysis thus shows the cultured ESCs under phase-contrast light microscopy (FIG. 5A). After 48 hours of transfection with the GFP gene, ESCs exhibited green fluorescence in more than 90% of transfected cells (FIG. 5B). In addition, GFP positive spots were detected under fluorescent microscopy in frozen sections prepared from MI hearts at 6 weeks after MI induction and cell transplantation (FIG. 5C). Moreover, engrafted cells were able to regenerate myocardial tissue in injured hearts.

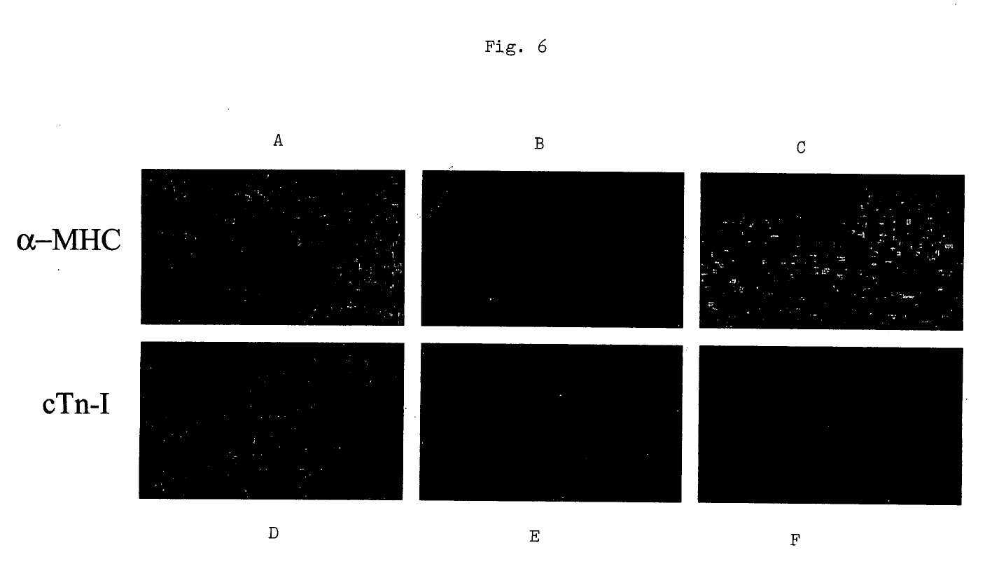

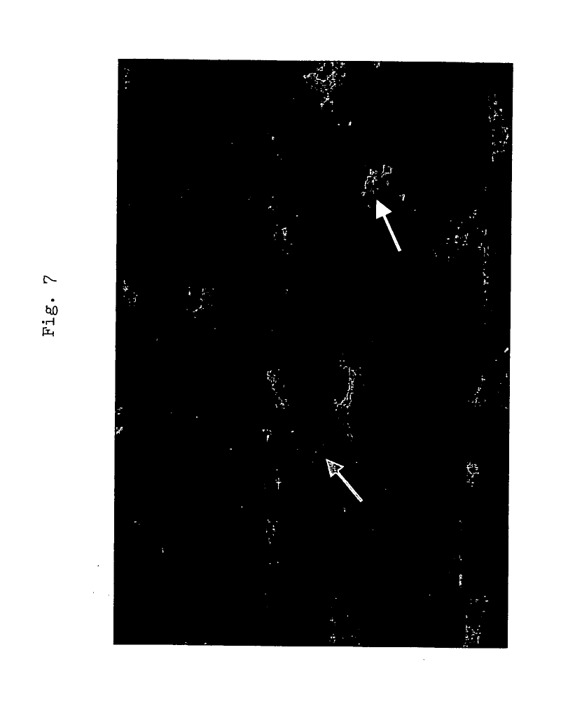

Subsequently, immunostaining for cTn-I and α-MHC in mouse hearts was conducted, as is shown by FIGS. 6A-6F. Upper panels (red): FIG. 6A shows immunofluorescent staining for α-MHC in sham-operated myocardium; FIG. 6B is the staining in infarcted myocardium with injection of the cell-free medium; and FIG. 6C shows the staining in infarcted myocadium with ESCs transplantation. Lower panels (green): FIG. 6D shows the immunofluorescent staining for cTn-I in sham-operated myocardium; FIG. 6E shows staining in infarcted myocardium with injection of the cell-free medium; and FIG. 6F shows staining in infarcted myocardium with ESCs transplantation.

Overall, FIG. 6 shows that intensive immunostaining for cTn-I and α-MHC was identified in normal myocardium with an even distribution (FIG. 6A) and in infarcted myocardium with ESCs transplantation (FIG. 6C). In contrast, immunostaining for both cTn-I and α-MHC was much lower in MI areas injected with the cell-free medium.

In addition, positive double staining for GFP and CX-43 was verified in injured myocardium with ESCs transplantation, as shown by FIG. 7. In contrast, both GFP and CX-43 were stained negatively in infarcted myocardium with medium injection (data not shown). These results demonstrate that engrafted cells not only survived in injured myocardium, but also formed new cardiac tissue.

FIG. 7 is a photograph of confocal microscopy of Immunofluorescence labeling of engrafted cells in injured myocardium (100×) with the antibodies recognizing GFP and CX-43. Double staining for GFP and CX-43 was carried out with the mouse anti-GFP and rabbit anti-CX-43 antibodies in myocardial frozen sections. GFP labeling was detected with a goat anti-mouse IgG conjugated to FITC and CX-43 labeling was detected with a goat anti-rabbit IgG conjugated to Texas Red. The arrows point the possible connection between GFP positive and negative cells (white arrow) and between GFP negative cells (yellow arrow).

### Experiment 4: Effects of Transplantation of ESCs Transfected with VEGF on Heart Function

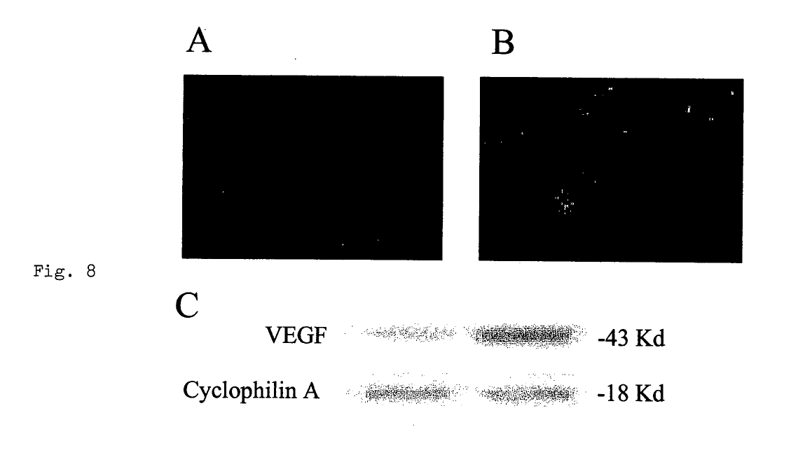

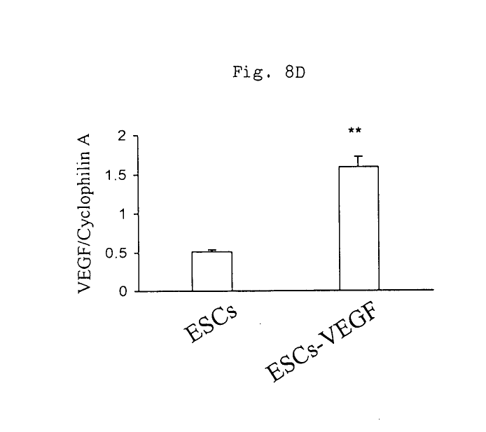

Application of VEGF is known to improve myocardial blood perfusion by an increase in collateral blood vessels in patients with myocardial ischemia. To test whether transplantation of ESCs overexpressing VEGF into injured myocardium improved cardiac function even greater, we transfected ESCs with a VEGF₁₆₅ cDNA and implanted ESCs with overexpression of VEGF to MI hearts. The results are illustrated by FIGS. 8A-8C respectively.

FIG. 8 as a whole shows the expression of VEGF in cultured ESCs with or without transfection of VEGF₁₆₅. VEGF was detected in cultured ESCs without transfection of the VEGF cDNA. FIG. 8A shows that the intensity of VEGF staining detected under fluorescent microscopy (×400) was markedly increased in cultured ESCs after 2 days of VEGF₁₆₅ transfection. FIG. 8C is a Western blot analysis of VEGF in cultured ESCs. The upper panels show the original VEGF and internal control (Cyclophilin A) blots for ESCs (left) and ESCs-VEGF (right) The lower panel is the normalized VEGF levels for ESCs (n=4) and ESCs VEGF (n=4). **, P<0.01, vs. ESCs.

The observed results thus show that cultured ESCs expressed a certain level of VEGF detected by immunofluorescent staining (FIG. 8A). After the human VEGF₁₆₅ gene was transfected into ESCs for 2 days, intensive immunofluorescence of VEGF in transfected ESCs was observed and indicated an overexpression of the growth factor (FIG. 8B). Western blot analysis further confirmed that VEGF was increased by 3-fold in cultured ESCs transfected with cDNA of VEGF (FIG. 8C).

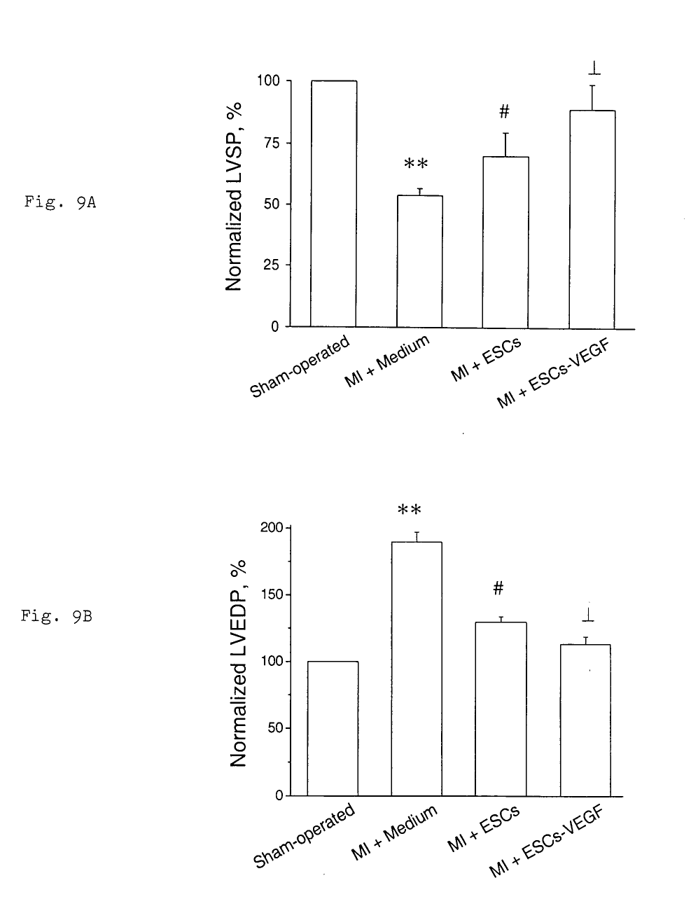

In addition, the experiments in vivo showed that the improvement of left ventricular function was significantly greater in the MI mice transplanted with ESCs plus VEGF than in the MI animals transplanted with ESCs alone. The differences of LVSP (P<0.05) and LVEDP (P<0.05) were statistically significant between the two group, as shown by FIGS. 9A and 9B.

FIG. 9 as a whole shows a comparison of left ventricular function in MI mice. Normalized left ventricular systolic pressure (LVSP, FIG. 9A) and normalized left ventricular end diastolic pressure (LVEDP,FIG. 9B) are shown in MI mice. Transplantation of ESCs alone (MI+ESCs, n=8) or ESCs-VEGF (MI+ESCs-VEGF, n=8) significantly improved left ventricular function compared the MI control animals (MI+Medium, n=7). The data was normalized to the values (100%) of the sham-operated animals (Sham, n=8). **, P<0.61, vs. Sham; #, P<0.05, vs. MI+Medium; 1., P<0.05, vs. MI+ESCs.

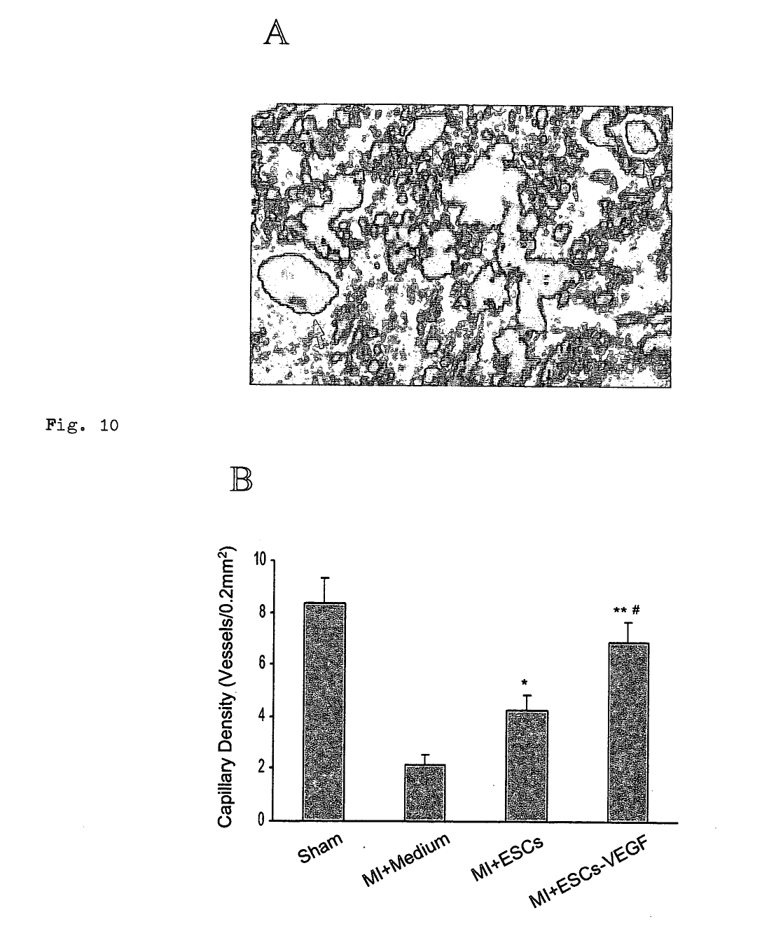

To assess the angiogenesis effect of VEGF, we calculated capillary density and compared the differences among animals with various treatments. The number of capillaries in injured myocardium with ESCs transplantation was significantly greater (P<0.01) than that in MI-untreated hearts. The capillary density in the ESCs-EVGF group was also significantly higher (P<0,01) than the control. In addition, the capillary density was significant higher in the ESCs-VEGF-transplanted mice than that in the animals transplanted With ESCs alone. This is shown by FIGS. 10A and 10B.

FIG. 10 as a whole shows the effects of ESCs transplantation on capillary density in infarcted myocardium. FIG. 10A reveals the HE staining of a myocardial section from a MI mouse heart transplanted with ESCs overexpressing VEGF. Arrows indicate the blood vessels in infarcted myocardium (magnification ×200). FIG. 10B graphically shows the averaged numbers of blood vessels for sham (n=8), MI+medium (n=7), MI+ESCs (n=8), and MI+ESCs-VEGF (n=7). **, P<0.01, vs. Sham; #, P<0.05, vs. MI+Medium; 1., P<0.05, vs. MI+ESCs.

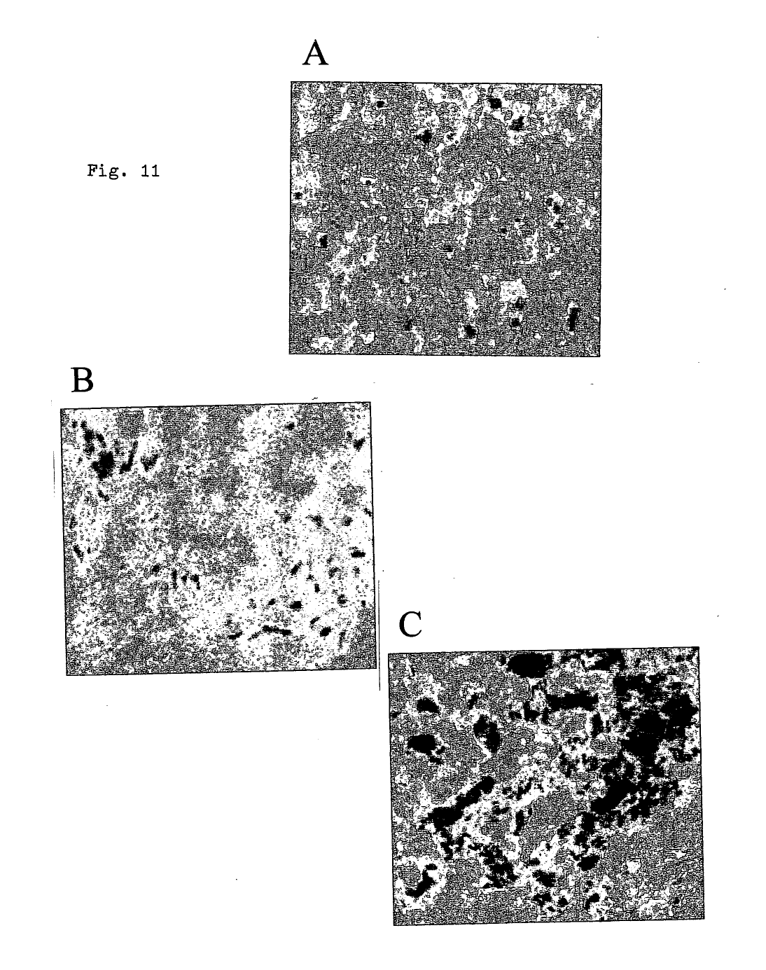

Subsequently, the effects of engrafted cells on neovascularization in the infarcted and peri-infarcted myocardium were further evaluated by immunohistochemical analysis. An anti-von Willebrand Factor (vWF, a marker of endothelial cells) antibody was applied to confirm new blood vessels in infarcted area. The results are shown by FIGS. 11A-11C respectively.

FIG. 11 as a whole reveals the positive immunostaining for blood vessel endothelial cells by anti-von Willebrand Factor (vWF) antibody in mouse myocardial sections. FIG. 11A shows Rich vWF staining (red) in a normal myocardial section demonstrates normal blood vessel distribution in a sham-operated mouse heart. FIG. 11B demonstrates that vWF staining was significantly reduced in infarcted myocardium injected with the cell-free medium. Sporadic vWF staining indicates few blood vessels in MI region; FIG. 11C shows that transplantation of ESCs with overexpression of VEGF significantly increased vWF staining in infarcted myocardium with numerous engrafted cells. Magnification ×200.

FIG. 11 clearly shows the red positive staining of vWF. Compared to normal myocardium (FIG. 11A), the density of vWF staining was dramatically decreased in infarcted myocardium sectioned from the MI heart injected with the cell-free medium (FIG. 11B). However, transplantation of ESCs with overexpression of VEGF markedly increased the density of vWF staining in infarcted myocardium with cell implantation. FIG. 11C clearly shows neovascularization in the infarcted area with engrafts of implanted cells. These results demonstrate that transplantation of ESCs with overexpression of VEGF produced a greater improvement of cardiac function in MI mice; and that the better outcome may result from a stronger angiogenesis effect.

### Conclusions Drawn from the Results of Experiment Series I

1. The empirical findings show that transplanted ESCs survived and differentiated into cardiomyocytes in injured myocardium and significantly improved left ventricular function in MI mice. The improvement of cardiac function was even greater in the MI hearts transplanted with overexpressing VEGF. In addition, ESCs themselves quantitatively expressed a sufficient amount of VEGF and were able to stimulate the growth of new blood vessels in injured myocardium. The angiogenesis effect was even stronger in infarcted myocardium when engrafted ESCs were transfected with VEGF to be expressed in-situ. Therefore, ESCs and their differentiated cells constitute and comprise important sources for cell therapy in patients with MI-induced heart failure in the future.

2. ESCs transplantation significantly improved left ventricular function and isometric contractility in post MI mice. One clinical possibility for the improvement of ventricular function is a reduction of infarcted area by regeneration of myocardium of engrafted ESCs. Reduction of infarct size could prevent over-stretching of the ventricle and preserve normal contractile function (Frank-Starling Law).

3. Myocardial regeneration by engrafted ESCs resulted in an improvement of global function in infarcted hearts, which could preserve papillary muscle function. Our morphological data confirm that the engrafted cells survived in infarcted myocardium by identification of GFP positive cells within implanted hearts 6 weeks after MI induction and cell transplantation. The intensive immunostaining for cTn-I and α-MHC in cell-transplanted MI hearts indicates differentiation and maturity of engrafted cells in injured myocardium. In contrast, both cTn-I and α-MHC were stained at a very low level in infarcted myocardium with medium injection. Furthermore, positive double staining for GFP and CX-43 in injured myocardium with ESCs transplantation indicates possible formation of morphological and functional connections among engrafted and host heart cells.

4. Another beneficial effect of ESCs transplantation is that engrafted ESCs induces angiogenesis in ischemically injured myocardium. In the present study, transplantation of ESCs alone or ESCs plus VEGF significantly increased the density of capillary blood vessels in infarcted myocardium. Thus, the improvement of left ventricular function in postinfarcted failing hearts after cell transplantation can result from regeneration of myocardium and blood vessels. Subsequently, this regeneration attenuated infarcted size and improved heart function.

5. Therapeutic angiogenesis markedly increases by combining application of ESCs and VEGF in infarcted myocardium. Our data show that ESCs-VEGF transplantation provided an even more effective approach to improve cardiac function in postinfarcted failing hearts. After intramyocardial transplantation, these cells communicate with their surrounding cells and tissues, signaling the formation of blood vessels to nourish them. The capillary density was significantly higher in the ESCs-treated MI animals than in the MI control mice. The difference between ESCs and ESCs-VEGF groups was significant. This difference enhances graft survival and account for the significantly greater improvement of ventricular function in the MI mice transplanted with ESCs-VEGF than in MI animals implanted with ESCs alone. In addition, this increase in capillary density is deemed to be significant for the life quality and life span in patients with heart failure.

6. Engrafted cells can restore damaged cardiac function. The present study demonstrates the beneficial effect of ESCs transplantation on cardiac function in MI animals. ESCs transplantation was not only able to regenerate injured myocardium, but also able to enhance neovascularization in infarcted area. As certain immunerelated cell surface proteins are not yet expressed in ESCs, another advantage of using ESCs for cell therapy is a decrease in immunorejection. Therefore, the improvement of cardiac function results from regeneration of cardiomyocytes and blood vessels in MI mice transplanted with ESCs alone or with ESCs overexpressing VEGF. This novel approach will provide the basis for future cell therapy for patients dying of MI and heart failure.

### Experimental Series II

Permanent loss of cardiomyocytes results in irreversible damage of cardiac function in patients who have suffered from myocardial infarction (MI). Replacing scar tissue of damaged heart with new functional cardiomyocytes is believed to improve myocardial contractility and prevent the progression of heart failure.

A number of recent studies demonstrated that transplantation of a range of different cultured cells into damaged myocardium was able to restore the impaired cardiac function in either cryoinjured or infarcted hearts. Some of these engrafted cells have been shown to survive, proliferate, and form gap junctions with the host myocardium. In contrast, other reported data found that fetal and neonatal pig cardiomyocytes and cardiac-derived cell line HL-1 did not survive after grafting into pig myocardium. The above discrepancies are believed to result from differences in the types of donor cells or the status of the host myocardium at the time of transplantation.

Embryonic stem (ES) cells are totipotent cells containing the capability of unlimited, in vitro proliferation useful to cardiogenesis. Our recent study showed that ES cell transplantation was feasible in infarcted rat myocardium and that improvement of heart function was observed in MI rats after 6 weeks of ES cell transplantation [Min et al., Circulation 102 (Suppl): 156, Abstract (2000)]. However, the long-term functional effects of ES cell transplantation on postinfarcted failing hearts remains to be determined. Therefore, this study was made to investigate the long-term effects of ES cell transplantation on mortality and the improvement of cardiac function in postinfarcted rats after 32 weeks of MI induction and cell implantation.

### Methods

### ES Cell Preparation for Transplantation

The mouse ES cell line, ES-D3, was obtained from the American Type Culture Collection (A TCC, Manassas, Va.) and maintained with the method as previously described (19). Briefly, ES-D3stem cells and their progenitor progeny cells were cultured in Dulbecco's modified Eagle's medium (DMEM) on mitotically inactive mouse embryonic fibroblast feeder cells (ATCC, Manassas, Va.). The medium was supplemented with 15% fetal bovine serum, 0.1 mM β-mercaptoethanol (Sigma, St. Louis, Mo.), and 10³ units/ml of leukemia inhibitory factor (LIF) conditioned medium (BRL, Gaithersburg, Md.) to suppress differentiation. To initiate lineage selection/commitment and differentiation, ES cells and their progenitor progeny cells were dispersed with trypsin and resuspended in the medium without supplemental leukemia inhibitory factor (LIF) and cultured with hanging drops (approximate 400 cells per 20 μl) for 5 days. They were then seeded into 100-mm cell culture dishes. The lineage-committed but undifferentiated beating cardiomyogenic clusters which resulted were then dissected by use of a sterile micropipette and re-cultured for another 2 days at 37° C. in a humidified atmosphere with 5% CO₂.

Before transplantation, cells were transfected with green fluorescent protein (GFP), a marker for identification of engrafted cells from host myocytes in injured myocardium. Plasmids with a hCMV IE promoter/enhancer diving GFP gene (5.7 kb) and Gene PORTER™ transfection reagent were obtained from Gene Therapy Systems Inc. (GTS Inc, San Diego, Calif.). An adequate amount of ES cells were plated in 100-mm dishes to obtain 50-60% confluence on the day of transfection. Two days after GFP transfection, cultured ES cells were trypsinized and resuspended in Joklik modified medium (Sigma, St. Louis, Mo.) with a density of 10⁷ cells/ml for cell transplantation.

### Experimental Myocardial Infarction and ES Cell Transplantation

Experiments were performed in male Wistar rats (Charles River, Wilmington, Mass.) with an initial body weight of ˜300 g. The investigation conformed to the Guide for the Care and Use of Laboratory Animals published by the US National Institutes of Health (NIH Publication No.85-23, revised 1996), and the protocol was approved by our Institutional Animal Care Committee. MI was created by ligation of the left anterior descending coronary artery as previously described (10). Shortly after induction of MI, 3×10⁵ ES cell suspension was injected into 3 sites in MI hearts with a tuberculin syringe. Two injection sites were at the border of ischemic area and one was in the middle of the infarcted region. Control animals received the same MI operation but were only injected with an equivalent volume of the cell-free medium.

The survival rate was evaluated in all groups during the whole process of experiments, i.e., 32-weeks follow up. The study was comprised of the following groups: MI rats transplanted with ES cells (MI+ES Cells, n=26); MI control rats injected with an equivalent volume of the cell-free medium (MI+Medium, n=31); and sham-operated rats with neither ligation of the coronary artery nor intramyocardial injection (Sham, n=23).

### Echocardiographic Studies and Measurement of Infarct Size

Thirty-two weeks after transplantation, the animals were anesthetized with pentobarbital. The echo cardiographic procedure was performed as previously described (8). A commercially available echocardiographic system equipped with a 12.5-MHz probe (Agilgent Sonos 5500) was used for all studies. Initially, a two-dimensional short-axis view of the left ventricle was obtained at the level of papillary muscles. After optimizing gain settings and ensuring that the image was on-axis, M-mode tracings were recorded through the anterior and posterior left ventricular (LV) walls at a paper speed of 100 mm/s. This orientation was chosen to allow delineation of wall thickness and motion in infarcted and noninfarcted territories. The data of M-mode tracings were analyzed from data recorded on an optical disk. LV mass was calculated using a standard cube formula. Relative anterior wall thickness, relative posterior wall thickness, and LV internal dimensions were measured from at least three consecutive cardiac cycles on a M-mode strip chart recording. Endocardial fractional shortening and midwall fractional shortening were also used as indices to estimate LV systolic function.

After echo cardiographic measurements, the rats were sacrificed to measure infarct size as described by Pfeffer et al. [Circ. Res. 57: 84-95 (1985)]. Infarct size was calculated by dividing the sum of the planimetered endocardial and epicardial circumferences of the infarcted area by the sum of the total epicardial and endocardial circumferences of the left ventricle.

### Measurement of Hemodynamics

In another series of experiments, rats were anesthetized again with pentobarbital after 32 weeks of MI operation and cell transplantation. Hemodynamic measurements in vivo were performed with the method as described previously [Orlic et al., Nature 410: 701-705 (2001)]. After hemodynamic measurements, the rats were sacrificed and the heart was rapidly excised. The left ventricle including the septum was weighed and normalized by body weight. The ratio was calculated as indices of hypertrophy.

### Identification of Transplanted Cells and Histological Studies

Subsets of animals were sacrificed at 32 weeks after ES cell transplantation; to evaluate the morphological characteristics and to identify the engrafted cells. The hearts were quickly removed and the free wall of the left ventricle, including the infarcted and periinfarcted regions, was embedded in tissue freezing medium for hematoxylin-eosin stain. Survival of transplanted cells was demonstrated by GFP positive engrafts with the frozen sections from infarcted rat myocardium. Regenerated cardiac-like cells from engrafted ES cells were verified by using an immunofluorescent method to identify cardiac specific proteins, cardiac myosin heavy chain (MHC) and troponin I (cTnI). Briefly, frozen tissue sections were fixed in acetone (4° C.) for 10 min, and incubated separately with a goat polyclonal IgG anti-TnI antibody (Santa Cruz Biotechnology Inc., Santa Cruz, Calif.) or a mouse anti-MHC monoclonal antibody (Berkeley Antibody Co., Richmond, Calif.) for 60 min at room temperature. After washing with PBS, sections were incubated with a rabbit anti-goat conjugated rhodamine IgG (for TnI) or a goat anti-mouse conjugated fluorescein IgG (for MHC) (Pierce Chemical Co., Rockford, Ill.). In addition, cardiomyocytes were .isolated using the Xiao et al. method [J. Physiol. (Lond) 508: 777-792 (1998)] from 32 weeks postinfarcted rat hearts to further verify the survival of engrafted cells.

### Measurement of Capillary Density

The effect of ES cell transplantation on angiogenesis was evaluated by counting the number of capillary vessels [Tomita et al., Circulation 100 (Suppl. II): II247-II256 (1999)], which is defined as the vessel diameter is less than 20 μm. The number of capillaries was counted under microscopy (×400 magnification) for five random fields in infarcted area and presented as the mean of blood vessels per unit area (0.2 mm²).

### Data Analysis

All values are presented as mean ±S.E. Data was evaluated by one-way analysis of variance (ANOV A) with repeated measurements. Difference between two individual groups was compared by using pair or unpaired Student's t-test. Survival rate during 32-week observation was analyzed by standard Kaplan-Meier analysis and a statistical comparison between survival curves was made using the log rank test. The criterion for statistical significance was set at the level of P<0.05.

### Empirical Results

### Experiment 5: Survival Rates

To evaluate the effect of ES cell transplantation on the mortality in MI animals, the survival rate during 32-week observation was calculated in three animal groups. The results are illustrated graphically by FIG. 12.

FIG. 12 shows: Kaplan-Meier survival curves for sham-operated rats, postinfarcted rats injected with the cell-free medium, and postinfarcted rats transplanted with ES cells during the 32-week trial. Sham, sham-operated rats; MI+Medium, postinfarcted rats with injection of the cell-free medium; MI+ES Cells, postinfarcted rats with transplantation of ES cells. ES cell transplantation significantly increased the survival rate compared to MI rats with injection of the cell-free medium during 32-week follow up.

FIG. 12 reveals that transplantation of ES cells significantly increased the survival rate in the MI rats. Only 3 out of 26 (11%) animals in this group died during the whole process of experiment. In contrast, the mortality was much higher in the post-MI rats injected with the cell-free medium. During the period of 32-week follow up, 7 out of 31 rats died in the MI+Medium group (23%). The Kaplan-Meier analysis showed a significant improvement (P<0.05) of survival rate in the MI rats transplanted with ES cells than the animals received intramyocardial injection of the cell-free medium.

### Experiment 6: Improvement of Left Ventricular Function

The effects of ES cell transplantation on left ventricular (LV) function and infarct area are shown in Table E2 for the MI rats after 32 weeks of MI operation and cell transplantation. LV weight and the ratio of LV weight to body weight were significantly increased in MI+Medium group compared to sham-operated rats. However, ES cell transplantation significantly reduced not only the severity of LV hypertrophy, but also the infarct size, in the MI rats with the 32-week follow up.

TABLE E2
General characteristics of sham-operated and MI rats

| | Sham | MI + Medium | MI + ESCs |
|---|---|---|---|
| | (n = 8) | (n = 7) | (n = 8) |
| BW (g) | 623.6 ± 14.2 | 619.5 ± 13.9 | 598.5 ± 16.4 |
| LVW (mg) | 978. ± 39.9 | 1224.3 ± 12.7* | 1143.6 ± 8.9^{#} |
| LVW/BW (mg/g) | 1.5 ± 0.15 | 2.1 ± 0.12* | 1.7 ± 0.11 |
| Infarct Size | | 37.2 ± 1.5 | 30.6 ± 1.1^{#} |

Values are presented as mean ± SE.
Sham, sham-operated rats 32 weeks after operation;
MI + Medium, postinfarcted rats injected with the cell-free medium 32 weeks after operation;
MI + ES Cells, postinfarcted rats with ES cell transplantation 32 weeks after operation.
BW, body weight;
LVW, left ventricular weight;
LVW/BW, ratio of left ventricular weight against body weight.
*P < 0.05, vs. Sham;
^{#}P < 0.05, vs. MI + Medium.

In addition, as shown by FIGS. 13A-13C, the LV systolic pressure (LVSP) and maximum arising rate of pressure (+dP/dtmax) significantly recovered in the postinfarcted rats transplanted with ES cells compared to the cell-free medium group. The LV end-diastolic pressure (LVEDP) was significantly smaller in cell-transplanted MI animals than in postinfarcted rats injected with the cell-free medium. These results indicate that ES cell transplantation significantly improved left ventricular function after 32 weeks of MI induction and cell implantation.

FIG. 13 as a whole demonstrates that ES cell transplantation significantly improved left ventricular function in postinfarcted rats using: Sham, sham-operated rats (n=10); MI+Medium, postinfarcted rats injected with the cell-free medium (n=9); MI+ES Cells, postinfarcted rats transplanted with ES cells (n=10). FIG. 13A presents LVSP, the left ventricular systolic pressure; FIG. 13B shows LVEDP, the left ventricular end-diastolic pressure; FIG. 13C identifies +dP/dtmax, the rate of peak left ventricular systolic pressure raise. Measurements were conducted at 32 weeks after MI operation.* P<0.05, ** P<0.01, vs. Sham; ^{#} P<0.05, vs. MI+Medium.

### Experiment 7: Echocardiographic Assessment

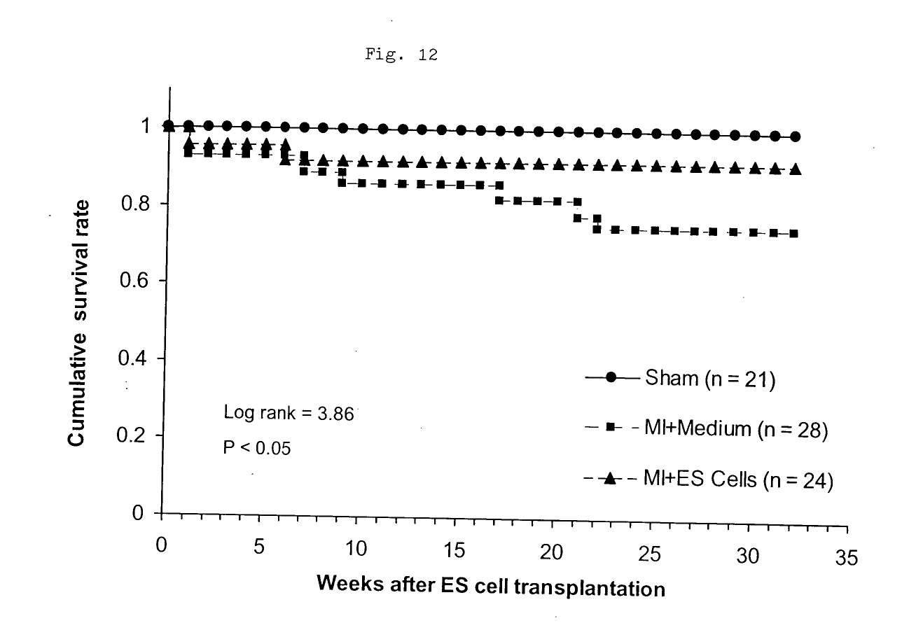

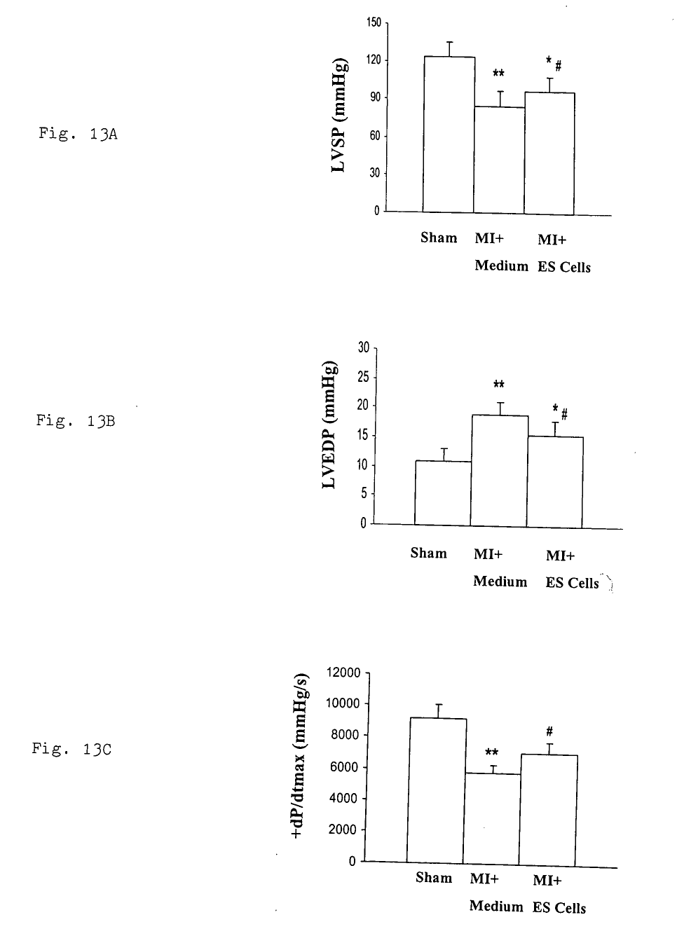

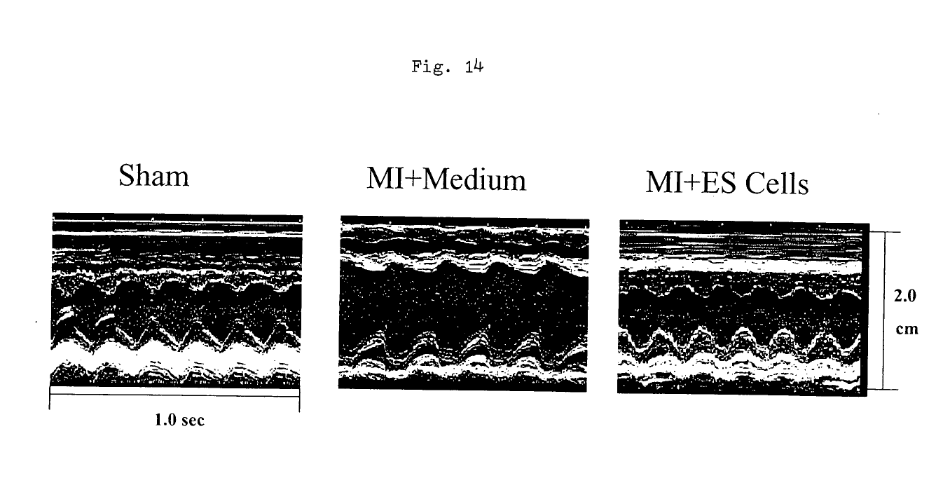

The results of echocardiographic assessment on cardiac function in MI rats after 32 weeks of ES cell implantation are shown by Table E3 and FIG. 14. The LV cavity became dilated in infarcted rat hearts with injection of the cell-free medium. Enlargement of LV cavity dimensions in infarcted hearts resulted in a significant decrease in anterior and posterior well thickness, and subsequently produced a reduction of endocardial and midwall fractional shortening. Transplantation of ES cells significantly attenuated the development of LV modeling with a lower ratio of LV mass/body weight 32 weeks after MI. LV anterior and posterior wall thicknesses were partially reserved in MI rats transplanted with ES cells. In addition, LV diastolic and systolic dimensions decreased in MI rats with cell transplantation.

FIGS. 14A-14C are representative echocardiographic recordings in a sham-operated rat (Sham), a postinfarcted rat with injection of the cell-free medium (MI+Medium), and a postinfarcted rat with transplantation of ES cells (MI+ES Cells). The anterior and posterior walls were thinned and hypokinetic, whereas the left ventricular cavity became dilated in the postinfarcted rat heart injected with the cell-free medium. In contrast, ES cell transplantation improved ventricular wall contractility and reduced the left ventricular dilation compared to MI hearts with injection of the cell-free medium.

TABLE E3
Echocardiographic measurements of left ventricular function in vivo.

| | Sham | MI + Medium | MI + ES Cells |
|---|---|---|---|
| PW th (%) | 79.2 ± 8.5 | 46.2 ± 3.5** | 59.5 ± 4*^{#} |
| AW th (%) | 64.4 ± 7.5 | 39.5 ± 4.5* | 54.6 ± 5^{#} |
| LVDd (mm) | 8.0 ± 0.2 | 10.5 ± 0.3** | 8.6 ± 0.3^{#} |
| LVDs (mm) | 5.1 ± 0.2 | 7.7 ± 0.4** | 6.2 ± 0.3*^{#} |
| En FS (%) | 36.7 ± 1.3 | 24.2 ± 1.2* | 31.0 ± 0.8^{#} |
| MW FS (%) | 21.1 ± 1.3 | 14.5 ± 1.1 | 17.6 ± 0.6 |
| LV Mass (g) | 0.92 ± 0.1 | 1.38 ± 0.2** | 1.09 ± 0.1^{#} |
| LV Mass/BW (mg/g) | 1.5 ± 0.1 | 2.3 ± 0.1** | 1.8 ± 0.3^{#} |

Values are presented as mean ± SE. Each group represents 5 rats.
PW th, relative posterior wall thickness;
AW th, relative anterior wall thichness;
LVDd, left ventricular diastolic dimension;
LVDs, left ventricular systolic dimesnion;
En FS, endocardial fractional shortening;
MW FS, midwall fractional shortening;
LV Mass/BW, ration of left ventricular mass against body weight.
*P < 0.01, vs. Sham;
^{#}P < 0.05, vs. MI + Medium.

### Experiment 8: Survival of Engrafted Cells in Damaged Myocardium

In order to identify the survival of engrafted cells in damaged myocardium, fresh frozen sections were prepared from postinfarcted hearts at 32 weeks after ES cell transplantation. These sections are illustrated by FIGS. 15A-15C respectively.

FIG. 15 as a whole reveals GFP positive spots and single cells from infarcted myocardium with cell transplantation. FIG. 15A illustrates that, under fluorescent microscopy (×200), the frozen section showed strong GFP positive tissue in cell-implanted myocardium after 32 weeks of MI induction and cell transplantation. FIG. 15B shows that single GFP positive cells were detected with fluorescent microscopy (×200) in cardiomyocytes isolated from a MI heart 32 weeks after cell transplantation. FIG. 15C corresponds to FIG. 15B, and shows GFP negative host cardiomyocytes. Both GFP negative and positive cells were rod-shaped with clear striations under light-contrast microscopy (×200). The insets of FIGS. 15B and 15C are the enlargement of the parts pointed by the arrows.

It will be noted that FIG. 15A shows that rich GFP positive spots were detected under fluorescent microscopy in one cell-engrafted myocardial slide. This result suggests that engrafted cells, at least part of them, survived well in injured myocardium after 32 weeks of MI induction and ES cell transplantation. In addition, GFP positive cells were observed in single myocytes isolated from infarcted hearts after 32 weeks of ES cell tranplantation (FIG. 15B). These GFP positive cells were rod-shaped with clear striations which are the characteristics of adult mammalian cardiomyocytes (FIG. 15C).

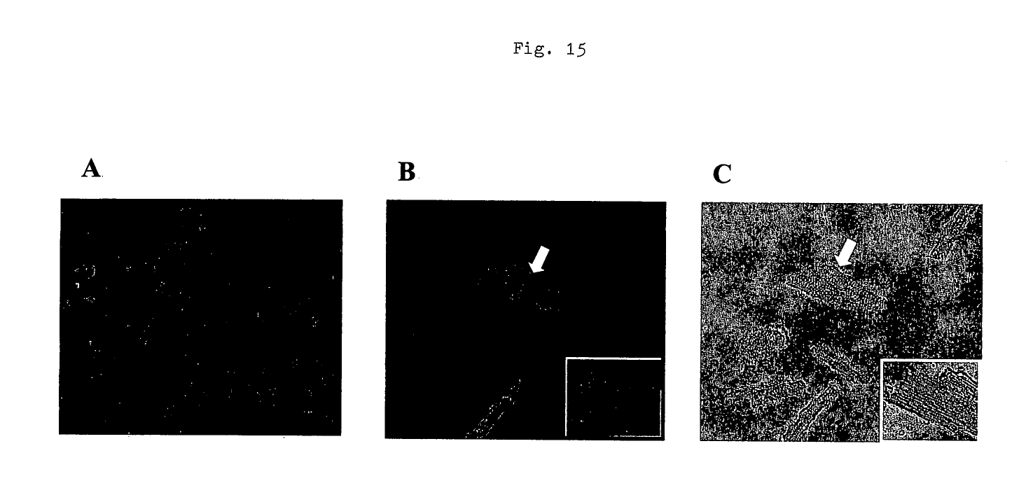

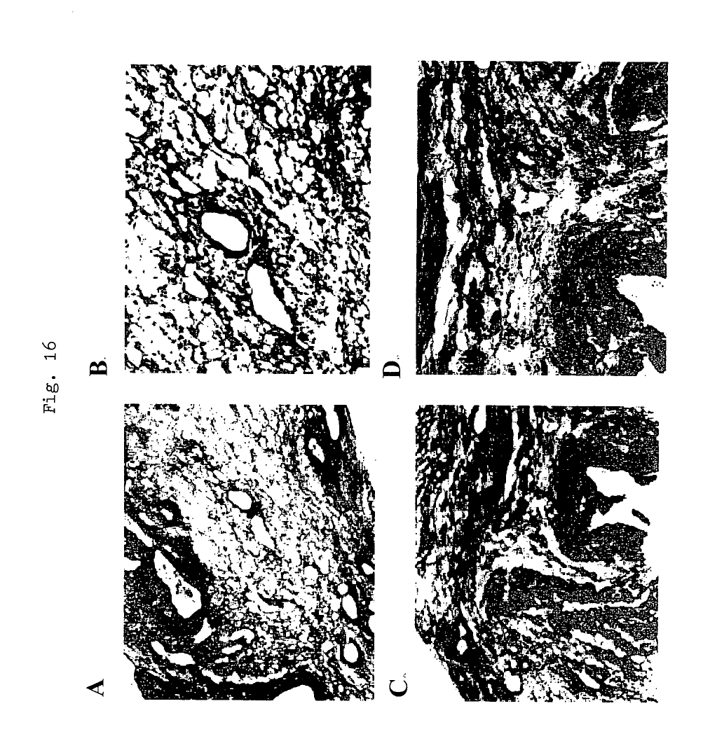

In addition, an analysis of myocardial sections was made with hematoxylin and eosin staining. These results are shown by FIGS. 16A-16H respectively.

FIG. 16 as a whole illustrates engrafted ES cells in postinfarcted rat myocardium which were identified with hematoxylin-eosin staining at 32 weeks after transplantation. FIGS. 16A and 16B show the lower (×40) and higher (×200) magnification, correspondingly, of infarcted myocardium with injection of the cell-free medium. FIGS. 16C and 16D show infarcted myocardium with transplantation of ES cells at lower (×40) and higher (×200) power, respectively. Engrafted cells are clearly seen in the infarcted area. FIGS. 16E and 16F reveal positive immunostaining for MHC (×200) and cTnI (×200) which was observed in infarcted myocardium transplanted with ES cells. In contrast, immunostaining for either MHC (FIG. 16E) or cTnI (FIG. 16F) was negative in MI myocardium with injection of the cell-free medium.

This hematoxylin and eosin staining of myocardial sections clearly showed engrafted cells in infarct areas of 32-week MI hearts with ES cell transplantation (FIGS. 16C and 16D). The engrafted cells in infarcted myocardium were stained positively to MHC and cTnI (FIGS. 16G and 16H). In contrast, scar tissue induced by MI with injection of the cell-free medium was stained negatively to MIC (FIG. 16E) and cTnI (FIG. 16F). These data demonstrate that engrafted ES cells not only survived in postinfarcted myocardium up to 32 weeks after cell transplantation, but also differentiated to mature cardiomyocytes. These new matured cardiomyocytes might play an important role in improvement of ventricular function in MI animals with ES cell transplantation.

### Experiment 9: Angiogenic Effects

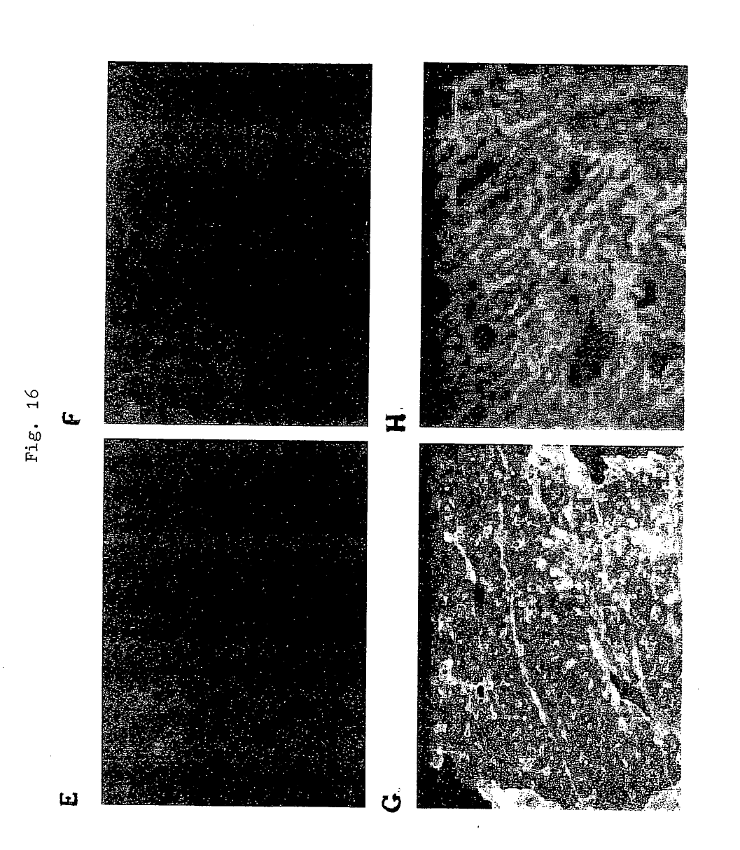

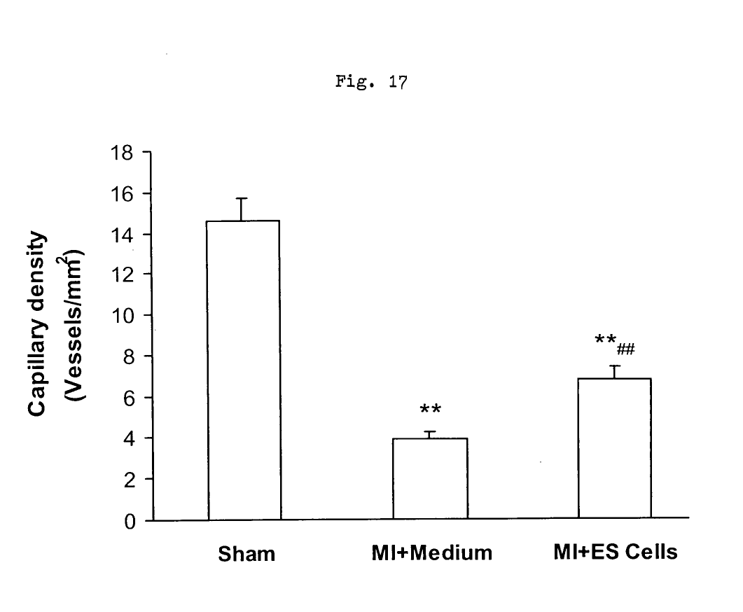

To examine whether transplanted ES cells induced an angiogenesis effects in injured myocardium, the density of blood vessels was calculated in infarcted area of MI hearts after 32 weeks of MI operation and cell transplantation. As shown by FIG. 17, the number of capillaries increased significantly (P<0.01) in damaged myocardium in MI rats with ES cell transplantation than in MI animals with injection of the cell-free medium. This result indicates that engrafted ES cells were able to cause or enhance neovascularization in injured myocardium.

FIG. 17 graphically shows the numerical density of capillaries (vessels/0.2 mm2) from sham-operated rat myocardium, infarcted rat myocardium with injection of the cell-free medium, and infarcted rat myocardium with transplantation of ES cells at 32 weeks after MI operation. ES cell transplantation significantly increased the capillary density compared to the infarcted myocardium with injection of the cell-free medium. ** P<0.01, vs. Sham;^{##} P<0/01, vs. MI+Control.

### Conclusion Drawn from the Empirical Data of Experimental Series II

1. The present results show that transplantation of ES cells improved cardiac function up to 32 weeks in postinfarcted rat hearts. These findings reveal that ES cell transplantation provides beneficial effects on heart performance for a prolonged period of time after MI. The data also demonstrate that implanted ES cells can form stable grafts and differentiate to cardiac-like cells in transplanted areas.

2. The present results show that transplantation of ES cells has the beneficial effects on cardiac function after MI for up to 32 weeks. Stable grafts in infarcted area were evidenced by strong GFP positive spots in MI rats after 32 weeks of MI induction and cell transplantation. Single GFP positive cells isolated from infarcted hearts with ES cell transplantation were striated and rod-shaped. In addition, immunostaining for the cardiac specific proteins, MIC and cTnI, was found in infarcted area of MI rats with ES cell transplantation. The data strongly support that improvement of cardiac function primarily results from cardiomyogenesis of implanted ES cells. Histologic examination did not show significant evidence of immunorejection of engrafted cells in MI hearts 32 weeks after cell transplantation. It is believed that the weak immunorejection in these experiments is due to the fact that ES cells express far fewer membrane surface antigens than do fully differentiated cardiomyocytes.

3. Angiogenesis also plays a role in improvement of heart function in rats after 32 weeks of MI induction and ES cell transplantation. Damaged myocardial regions with enriched grafted cells were accompanied with new blood vessels found in our experiments. Therefore, the angiogenesis effect is crucial to the survival of transplanted cells. New blood supply to damaged myocardium may provide nutrition to implanted cell and also rescue some host myocytes injured in ischemic area. These effects will help the heart to improve its function. The initiation of the angiogenesis effect is caused directly by ES cells.

4. The present results demonstrate the long-term beneficial effects on progressive ventricular remodeling and improvement of damaged heart function after ES cell transplantation in MI rats. Differentiated myocytes and the angiogenesis effect from engrafted cells may result in the functional improvement of MI hearts after 32 weeks of cell implantation. Therefore, this unique approach offers clinical value for the treatment of myocardial infarction and heart failure.

5.

### Experimental Series III

### Background

Stem cells have traditionally been characterized as either embryonic or organspecific. Several studies suggest that the latter can transdifferentiate into other cell types, including myocytes and neurons, carrying significant implications for the possible clinical use of these cells. After local transplantation, heart cells and myoblasts show significant potential to repair infarcted myocardium and improve heart function. It has recently been demonstrated that stem cells (bone marrow cells) have the potential to regenerate infarcted myocardium. Other studies have shown that human bone marrow-derived angioblasts accelerate the neovascularization of ishemic myocardium and improve myocardial function.

Myocarditis is a multifacted disease process involving viral infection, immune activation, and microvascular spasm. It is characterized by myocardial necrosis and inflammation in the acute stage, followed by myocardial fibrosis, calcification, cardiac dilatation, hypertrophy and heart failure in the chronic stage. Although the specific cause of myocarditis in any given patient often remains unclear, a large variety of infections, systemic diseases, drugs, and toxins have been associated with the development of this disease.

There is also a consensus that viruses may be a predominant cause of myocarditis. For example, human immunodeficiency virus (HIV) has been detected in heart tissue from patients with the acquired immunodeficiency syndrome. Hepatitis C is able to replicate in the human myocardium and may also be involved in the development of dilated cardiomyopathy. Other viral agents such as influenza A and B, polio, mumps, and rubella have been shown to cause myocarditis.

At present, there are no known effective therapies for preventing or reversing the structural and functional changes that occur and may cause death or debility of approximately one third of patients with clinical manifestations of myocarditis. Nevertheless, we believe that ES cells are capable of responding to signals from areas of inflammation and necrosis in myocarditis by proliferating, migrating into, and restoring functionality of damaged areas. To demonstrate this capability empirically, mice were injected with mouse embryonic stem cells via tail vein and immediately inoculated with encephalomyocarditis virus (EMCV). The morbidity, mortality, and histopathology of these mice were then assessed to evaluate the effect of ES cells on viral myocarditis.

### Materials and Methods

### Preparation of ES Cells

The mouse ES cell line, ES-D3, was obtained from the American Type Culture Collection (ATCC, Manassas, Va.) and maintained as stem cells and progenitor progeny cells with the methods as previously described. Briefly, ES-D3 cells were cultured in Dulbecco's Modified Eagle's medium (DMEM) on mitotically inactive mouse embryonic fibroblast feeder cells (ATCC, Manassas, Va.). The medium was supplemented with 15% fetal bovine serum, 0.1 mM β-mercaptoethanol (Sigma, St. Louis, Mo.), and 10³ units/ml of leukemia inhibitory factor (LIF) conditioned medium (BRL, Gaithersburg, Md.) to suppress differentiation.

Before cell transplantation, however, ES cells were transfected with enhanced green fluorescent protein (GFP), used to identify survival of transplanted ES cells. A plasmid with hCMV IE promoter/enhancer diving GFP gene (5.7 kb) and GenePORTER™ transfection reagent was obtained from Gene Therapy Systems Inc. (GTS Inc., San Diego, Calif.). An adequate amount of ES cells and their progeny cells were plated in 100-mm dishes to obtain 50-60% confluent on the day of transfection. Plasmid GFP DNA (8 μg) was added to each dish with a calcium phosphate precipitation method. After 2 days of GFP transfection, cultured ES cells were trypsinized and resuspended in Joklik modified medium (Sigma, St. Louis, Mo.) with a density of 10⁷ cells/ml for the use of infusion.

### Virus Preparation and Inoculation of Mice

The M variant of encephalomyocarditis virus (EMCV) (ATCC, Manassas, Va.) was used in this study. Viral stock was prepared as described. Briefly, human amnion (FL) cell monolayers were infected with EMCV and harvested when cytopathic effects were completed. The viral titers were determined by plaque formation on FL cell monolayer. The viral stock was stored at −80° C. until use. Mice were inoculated intraperitoneally (ip) with 140 plaque-forming units (0.1 ml) of EMCV diluted in Eagle's minimum essential medium.

### Experimental Protocol

A total of 100 4-week-old BALB/c male mice were obtained from the Charles River Laboratories (Wilmington, Mass.). ES cells were administered via tail vein (1×10⁷ cell per mouse). The mice were immediately inoculated with EMCV after the administration of ES cells. Animals were randomly divided into five groups: medium control, ES cells, EMCV, EMCV+medium, and EMCV+ES. The day of virus inoculation was defined as day 0. Animals were sacrificed at 1, 3, 7, 14, 21, and 30 days following virus inoculation to determine pathological changes at each stage of infection. These times were chosen to coincide with the acute phase (day 3), initial myocardial inflammation (day 7), peak inflammatory cell infiltration (day 14), and the beginning of fibrosis and dilated cardiomyopathy (day 21 and 30).

All animal experiments were performed in accordance with National Institute of Health guidelines. Protocols were approved by the Animal Care and Use Committee of Beth Israel Deaconess Medical Center and Harvard Medical School.

### Histopathology and GFP Fluorescence

At each designed day, 4 mice were randomly chosen for sacrifice. Heart, spleen, liver, lung, and kidney were excised and embedded in tissue-freezing medium (Triangle Biomedical Sciences, Durham, N.C.) and then frozen in liquid nitrogen. The tissues were then cryostat-sectioned to 5 μm thickness. Those sections were kept at −80° C. until GFP detection or pathology score was measured by hematoxylin and eosin staining. Several sections of each organ were scored blindly. For each myocardial sample (dead mice did not undergo necropsy), histological evidence of myocarditis and inflammation was classified in terms of the degree of cellular infiltration and myocardial cell necrosis and graded on a five-point scale ranging from 0 to 4+. A zero score indicated no or questionable presence of lesions in each category. A 1+ score described a limited focal distribution of myocardial lesions. A score of 2+ to 3+ described intermediate severity with multiple lesions, whereas a 4+ score described the presence of coalescent and extensive lesions over the entire examined heart tissue.

### Statistical Analysis

Results are presented as mean ±SD. Survival of mice was analyzed by the Kaplan-Meier methods. Comparison within and between groups was performed using one-way ANOVA. Post hoc comparison of individual groups was done using Newman-Keul subgroup analysis. A P value of<0.05 was considered significant.

### Empirical Results

### Experiment No. 10: Morbidity and Mortality

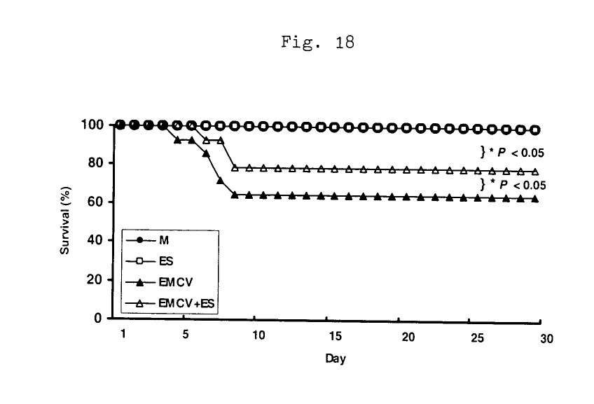

Infection with EMCV produced similar pathological changes in all mice under test. In brief, 3 days after virus inoculation, the mice appeared ill, and some developed coat ruffling, weakness, and irritability. Some mice developed a paralysis of the hind legs. Mortality among the virus control mice was 36%. However, in the ES cell group, the mortality of myocarditis mice was 20% (P<0.05 compared to the untreated group). These results are graphically illustrated by FIG. 18.

FIG. 18 shows the survival of myocarditis mice. Animals were treated with ES cells via tail vein. The percent survival in the ES cells-treated groups was significantly higher than that in the V alone group. * P<0.05. M: medium; EMCV: virus; ES: mouse embryonic stem cell; EMCV+ES: virus plus stem cell.

### Experiment No. 11: Histological Examination

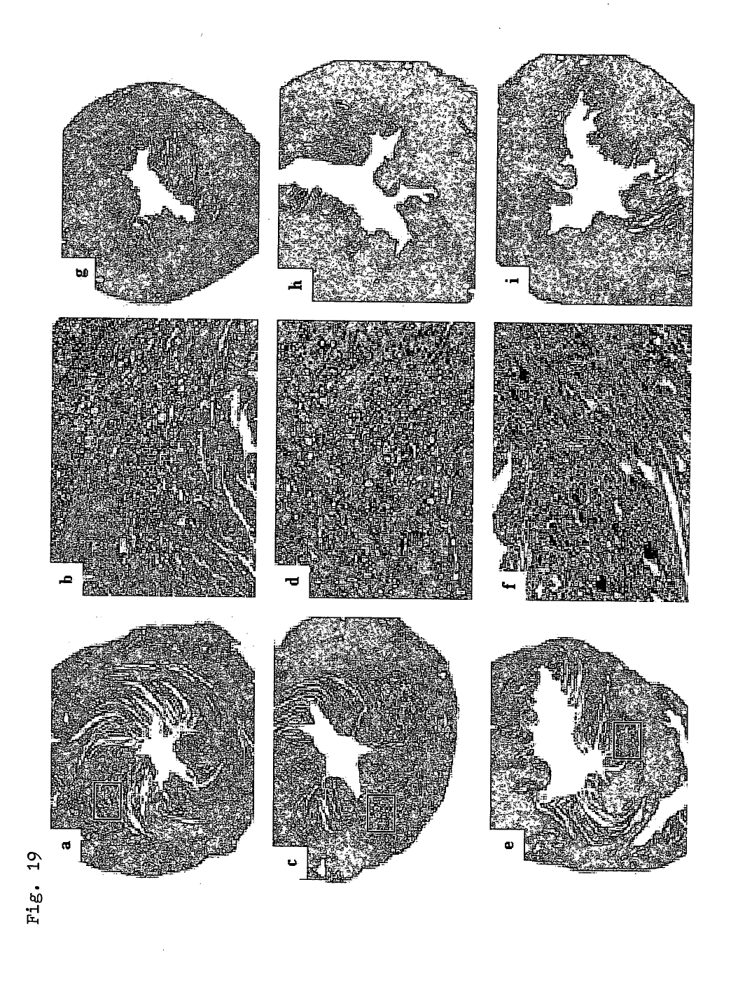

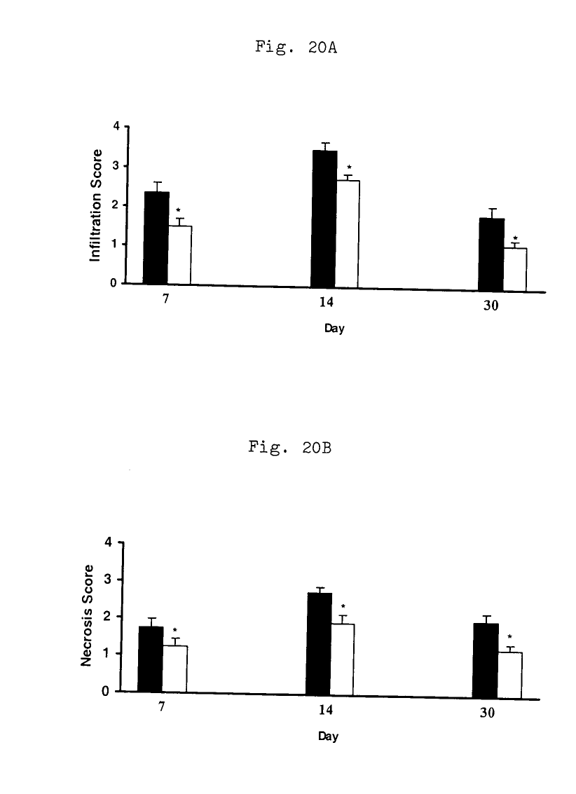

On day 3, a few scattered foci of myocyte necrosis associated with inflammatory cells were noted in myocardium from viral infected mice. On day 7, 14, and 30, myocyte necrosis and accompanying inflammation had become extensive, confluent in some areas and multifocal in others, as shown by FIGS. 19A-19I. In the ES cell group, pathological changes were ameliorated among myocarditic animals. The histopathological scores of necrosis and inflammatory cell infiltration were significantly lower in the ES cell groups in comparison with those of untreated animals, as shown by FIGS. 20A and 20B.

FIG. 19 as a whole provides photomicrographs (light microscopy) of hematoxylin and eosin-stained myocardial sections from ES cells-treated myocarditis mice. In virus control hearts there is inflammatory cell infiltration and necrosis at day 7 (FIGS. 19A and 19B; low- and high-power magnification, respectively); at day 14 (FIGS. 19C and 19D) and at day 30 (FIGS. 19E and 19F), extensive infiltration and necrosis, as well as fibrosis and calcification; photomicrographs of stem cells treated myocarditis hearts at day 7 (FIG. 19G), at day 14 (FIG. 119H), and at day 30 (FIG. 19I). The arrows point to infiltration and necrosis. Low magnification: original ×40; high magnification: original ×200.

FIG. 20 as a whole graphically illustrates the histological grading of infected mouse hearts. Histological scoring of hearts ranged from 0 to 4⁺ in each of the categories of inflammation and necrosis. * P<0.05 compared to virus group. Open bar: EMCV group, Solid bar: EMCV plus ES cells group. FIG. 20A illustrates infiltration and FIG. 20B illustrates necrosis.

### Experiment 12: ES Cell Migration into the Heart

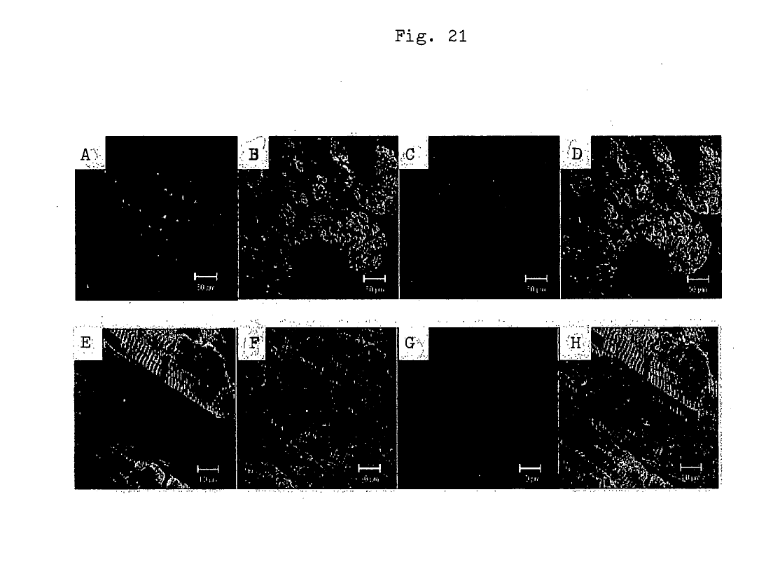

ES cells are demonstrated to migrate into the myocardium directly. FIG. 21 as a whole provides myocardial sections (confocal microscopy) from viral myocarditic myocardium treated with embryonic stem cells at day 14 after infection. FIG. 21A shows minimal green fluorescence in myocardium from uninfected mice treated with ES cells; FIG. 21B shows GFP (green) in necrosis area of myocarditic myocardium; FIG. 21C shows DAPI staining (blue); FIG. 21D shows the overlap of FIGS. 21B and 21C; FIGS. 21E and 21H are higher magnification of GFP (green) in myocarditis heart; α-actinin (red); DAPI (blue); and an overlap of FIGS. 21E, 21F and 21G.

In particular, it is noted that FIG. 21A shows there is minimal green fluorescence in myocardium from uninfected mice treated with ES cells transfected with cDNA for GFP. In contrast, FIG. 21B indicates that there is significant green fluorescence 14 days after administration of ES cells in myocardium from viral infected mice, indicating that ES cells homed to the hearts of animals with myocarditis. FIGS. 21E-21H respectively indicate that ES cells can differentiate into myocytes.

### Conclusions Drawn from the Empirical Results

1. Administration of mouse ES cells significantly decreased the mortality of viral myocarditis. This is the first demonstration of a protective effect against viral myocarditis by ES cell transplantation. The results clearly demonstrate that: (a) ES cells significantly increased the survival of mice with myocarditis; (b) invasion of the virus stimulated the migration of ES cells into the myocardium; and (c) ES cells differentiate into myocytes in the myocarditic heart.

2. Regeneration of organ-specific new cells may be one of the major mechanisms used by stem cells to repair damaged tissue and improve function. The present study shows that ES cells, which were administered via tail vein, significantly decreased the mortality, necrosis and infiltration of inflammatory cells. The GFP and α-actinin fluorescence studies indicated that ES cells migrated into the myocarditic heart and differentiated into new myocytes. Thus, the differentiation of myocytes is deemed to be one of the major reasons that ES cells decrease the necrosis, inflammation and mortality of viral myocarditis.

3. Current theories about myocarditis suggest that a three-step process is involved. The process begins with an acute phase 0-3 days after viral infection. There is an initial viral infection of genetically susceptible host myocytes that can lead to early pathological evidence of myocardial damage. After viral invasion of the myocardium, cell-mediated immunity activates the subacute phase of myocardial and endothelial damage. Finally, a chronic phase is characterized by slow myocyte loss and myocardial fibrosis, which progresses to dilated cardiomyopathy, terminal failure and death. The principal pathogenic mechanisms probably include direct viral tissue damage and immunocyte-mediated cellular damage critical for the progression of myocarditis to dilated cardiomyopathy and heart failure.

During this pathological period, positive GFP staining was empirically detectable in the myocardium, but there was no significant pathological difference between the treated and untreated groups. Accordingly, it is concluded that ES cells do not affect this phase. The immune T-cell infiltration peaked on day 7 to 14 after virus inoculation, coinciding with the most severe acute pathological damage in the myocardium. In the period from day 7 to 14, mortality reached its peak and subsequently declined.

Furthermore, the scores of myocardial necrosis and inflammatory cell infiltration at day 7 and 14 were higher than at other time points, and GFP and α-actinin were detectable from days 7 to 30 after ES cell administration and virus inoculation. Based on these results, it is also concluded that ES cells migrate from an extra-cardiac source to the heart in order to protect it against myocarditis.

4. ES cells are believed to have to ways to rescue inflammatory or damaged tissue: (a) differentiation into new myocytes to replace dead or damaged cells and (b) delivery of pharmacologically active substances to affected areas of the heart. It is also possible that when myocardial injury or inflammation occurs, the affected tissue may release a signal, which stimulates stem cells to migrate toward the myocardium. However, at this time the determination of the detailed mechanism of cellular homing remains unknown.

### Experimental Series IV

### Background

As a result of limited myocyte regenerative capabilities, injured myocardium cannot prevent the onset and evolution of ventricular dysfunction. Although limited regeneration of cardiomyocytes has recently been reported in human infarcted hearts, it is generally acknowledged that dead myocardium is replaced by nonfunctional fibrous tissue. High morbidity of ischemic cardiac dysfunction and shortage of donor hearts demand a constant search for new approaches to treat heart failure. Cell transplantation, including use of AT1, fetal or neonatal cardiomyocytes, satellite cells and bone marrow cells each have been demonstrated to be of some therapeutic value for the repair of damaged myocardium in animal models.

The bone marrow is known to contain a population of rare progenitor cells known as mesenchymal stem cells (MSCs), which have the capability to colonize different tissues, replicate, and differentiate into multilineage cells, including cardiac muscles. Human MSCs have been demonstrated to have the ability to proliferate extensively, and maintain the ability to differentiate into multiple cell types in vitro. A recent study showed that the implanted bone marrow cells can differentiate into myocytes and coronary vessels to ameliorate the function of the injured heart.

In order to provide important clinical insights for cell therapy, the present study investigated the feasibility of transplantation of hMSCs alone or co-transplantation (1:1) of hMSCs plus human fetal cardiomyocytes (hFCs) in MI pigs. Hemodynamic evaluation of ventricular function and histologic evidence of engrafted cells in injured myocardium were evaluated and compared in MI pigs with or without cell transplantation.

### Methods

### Cell Culture and Preparation

The hMSCs and hFCs (19 weeks) were obtained from BioWhittaker Inc. (Walkersville, Md., USA) and maintained with the method as previously described [Smith, A. G., J. Tissue Culture Methods 13: 89-94 (1991)]. Briefly, a Bullet-kit containing mesenchymal growth supplements was used to culture cells. Before cell transplantation, hMSCs were transfected with enhanced GFP cDNA for identification of the survival of engrafted cells. Approximately 3×10⁵ of hMSCs were plated in 100-mm dishes and cultured to obtain ˜90% confluence on the day of transfection. Two days after GFP transfection, cultured hMSCs were trypsinized and centrifuged. Collected hMSCs and hFCs were resuspended in the cultured medium for cell transplantation with a concentration of 10⁷ cells/ml.

### Experimental Animals and Surgical Preparations

MI was performed in male Yorkshire pigs with a body weight of ˜15 kg. The animals were sedated with ketamine (15 mg/kg, i.m.) and thiopentathal (5 mg/kg, i.v.). After tracheal intubation, animals were ventilated with ˜2% isoflurane at a rate of 12 breaths/min (Hallowell EMC Model 2000, Veterinary Anesthesia Ventilator, Pittsfield, Mass., USA). Electrocardiogram and respiration were monitored by a multiple-channel recorder (Portal Systems Inc., Beaverton, Oreg., USA). The right femoral artery was isolated and cannulated with an introduction sheath. Through the catheter sheath, a 7 Fr. pig-tailed catheter was retrogradely advanced into the left ventricle. The catheter was connected to a pressure transducer and intraventricular pressure was recorded by a chart-strip recorder. The LVSP, LVEDP, +dP/dt, and −dP/dt were measured to evaluate ventricular function.

The heart was exposed by means of a left thoracotomy through the 4th and 5th intercostal space. The distal end, just below the 3rd diagonal, of the left anterior descending coronary artery was ligated. Five minutes after ligation, 7×10⁶ cells of either hMSCs plus hFCs (1:1,10⁷ cells/ml) or hMSCs alone (10⁷ cells/ml) was injected into the border area of ischemic myocardium. Control MI hearts received the same MI operation, but were injected with the same volume of the cell-free medium. Antibiotics (Cefazolin, 35 mg/kg, i.m.) and analgesics (Buprenex, 0.03 mg/kg, i.m.) were administered shortly after surgery. Cefazolin (35 mg/kg) was maintained daily for 5 days after operation. Cyclosporine A, 15 mg/kg, was given orally every other day until the animals were sacrificed.

### Assessment of Cardiac Function and Myocardial Blood Flow

Ventricular function was assessed by echocardiography as described previously [Litwin et al., J. Am. Coll. Cardiol. 28: 773-781 (1996)]. Briefly, a commercially available echocardiographic system equipped with a 12.5-MHz probe (Agilgent Sonos 5500) was used and the results were analyzed from data recorded on an optical disk. Direct measurement of hemodynamics was conducted by intraventricular catheterization in MI pigs before ligation (baseline values), one hour, and 6 weeks after MI induction and cell implantation.

Six weeks after cell transplantation, stable isotope-labeled microspheres (BioPAL Inc., Worcester, Mass., USA) were use to determine coronary blood flow [as per Reinhardt et al., Am. J. Physiol. 280: H108-H1 16 (2001)] in anesthetized MI pigs under resting condition or with pacing stress by electric stimulation, 180 beats/min. In brief, a set of microspheres (2×10⁶) were diluted in 3 ml of sanSaLine™ saline (BioPAL Inc., Worcester, Mass. USA) and injected into the left atrium over 30 seconds. Reference blood samples were withdrawn by using a syringe pump at a constant rate of 5 ml/min through the femoral artery to calculate absolute myocardial blood flow. Finally, the heart was excised and regional myocardial blood flow was determined by BioPAL Inc., where collected heart tissues and blood samples were exposed to a field of neutrons.

### Morphology and Histology of Infarcted Myocardium

Subsets of animals were sacrificed after assessment of hemodynamics and blood flow 6 weeks after MI. The hearts were quickly removed, and selected tissues from the free wall of the left ventricle, including infarct and peri-infarct regions, were embedded in tissue freezing medium (Fisher Scientific, Fair Lawn, N.J., USA). Frozen sections (8 μm in thickness) of left ventricular tissue were made for identification of implanted cells and for immunofluorescent staining. Other hearts were fixed in 10% formalin overnight. The cardiac tissues were paraffin-embedded and sectioned at 5 μm thickness for hematoxylin and eosin staining. GFP positive spots under fluorescent microscopy represented the presence of engrafted cells in injured myocardium. Immunostaining for a-myosin heavy chain (α-MHC) and cardiac troponin I (cTnI) identified the survival and differentiation of engrafted hMSCs and hFCs. Frozen sections were washed three times in PBS and incubated with Cy3conjugated goat anti-mouse IgG antibody (Sigma, St. Louis, Mo., USA) for 45 min. Nonspecific binding was blocked by incubation with 1% bovine serum in remaining sections. Different frozen sections were stained immunohistochemically with a mouse monoclonal anti-GFP antibody (Zymed, San Francisco, Calif., USA), a goat polyclonal IgG anti-cTnI antibody (Santa Cruz Biotechnology Inc., Santa Cruz, Calif., USA), or a mouse anti-α-MHC monoclonal antibody (Berkeley Antibody Co., Richmond, Calif., USA) for 60 min. After washing with PBS, sections were incubated with a rabbit anti-goat conjugated rhodamine IgG (for cTnI) or a goat anti-mouse conjugated fluorescein IgG (for A-MHC and GFP) antibody (Pierce Chemical Co., Rockford, Ill., USA). Finally, fluorescent staining for α-MHC and cTnI were detected and photographed under fluorescent microscopy.

### Data Analysis

All values are presented as mean ±SE. The data collected before and after cell transplantation were compared by the paired Student's t test in each group. ANOVA was used for the comparison of the differences among the data delivered from groups more than two. P<0.05 was considered as significant difference.

### Empirical Results

### Experiment No. 13: Improvement of Cardiac Function

Experimentally, twenty-five animals received permanent ligation of the left coronary artery just below the third diagonal branch. Five animals died of lethal ventricular arrhythmias within 24 hours after MI operation. The study was comprised of the following groups: MI pigs transplanted with hMSCs alone (MI-hMSCs, n=6); MI pigs co-transplanted with hMSCs and hFCs (MI-hMSCs+hFCs, n=7); and MI control pigs injected with an equivalent volume of the cell-free culture medium (MI-Control, n=7). Baseline ventricular function before induction of MI assessed by hemodynamic measurements was not significantly different among the three groups. Myocardial infarction decreased cardiac function reflected by reduction of the left ventricular systolic pressure (LVSP), peak rate of the left ventricular systolic pressure rise (+dP/dt), and peak rate of the left ventricular systolic pressure fall (−dP/dt). Additionally, the left ventricular end-diastolic pressure (LVEDP) was elevated in all infarcted animals compared to their pre-MI values.

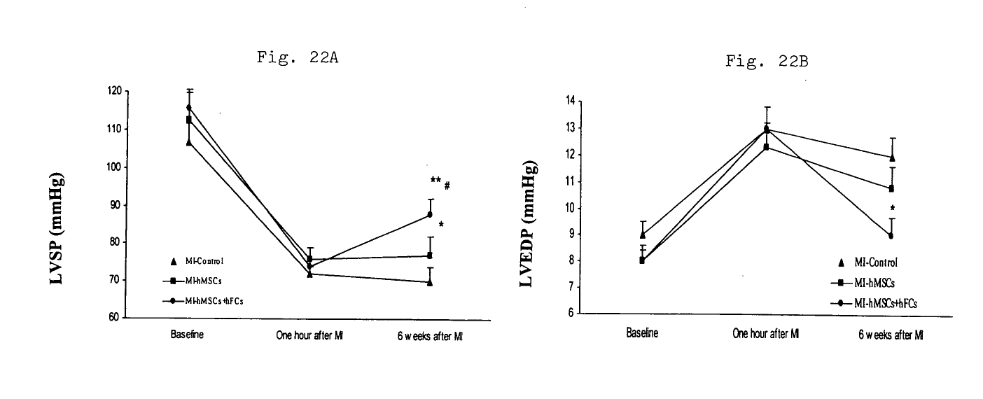

Six weeks after MI operation, cell transplantation significantly improved the ventricular function by reducing the LVEDP and increasing LVSP, +dP/dt and −dP/dt, as illustrated by Tables E4, E5 and FIG. 22. Moreover, the beneficial effects of cell transplantation on cardiac function were even greater in MI pigs co-transplanted with hMSCs plus hFCs. The increased improvement in cardiac function in co-transplanted animals was persistent when the MI hearts were paced at a rate of 180 beats/min—as shown by Tables E6 and E7.

TABLE E4
Hemodynamic study in porcine hearts before cell transplantation
(Baseline values)

| | | |
|---|---|---|
| LVSP | LVEDP | +dP/dtmax |

Cell transplantation was performed shortly
after MI and followed 6 weeks

| | | | |
|---|---|---|---|
| Control-1 | 108.6 ± 2.1 | 4.5 ± 0.3 | 2248 ± 109 |
| (n = 7) | | | |
| hMSCs + hFCs | 103.7 ± 1.7 | 4.5 ± 0.3 | 2146 ± 50 |
| (n = 6) | | | |
| hMSCs alone | 119.4 ± 1.8 | 4.8 ± 0.3 | 2160 ± 33 |
| (n = 7) | | | |
| hMSCs + hFCs | 119.4 ± 1.8 | 4.8 ± 0.3 | 2382 ± 74 |
| without cyclosporine | | | |
| (n = 6) | | | |

Cell transplantation was performed 2 weeks
after MI and followed 6 weeks

| | | | |
|---|---|---|---|
| Control-2 | 118.8 ± 0.7 | 6.8 ± 0.9 | 2100 ± 82 |
| (n = 5) | | | |
| hMSCs + hFSc | 116.3 ± 1.9 | 5.7 ± 0.7 | 2172 ± 69 |
| (n = 6) | | | |

Values are mean ± SE.
Control-1, post-MI pig hearts transplanted with medium performed shortly after MI;
hMSCs + hFCs, post-MI pig hearts co-transplanted with hMSCs plus hFCs and administered with cyclosporin A;
Control-2, post-MI pig hearts transplanted with medium performed 2 weeks after MI.

TABLE E5
Hemodynamic study in post-MI porcine hearts after cell transplantation
(6 weeks after cell transplantation)

| | | |
|---|---|---|
| LVSP | LVEDP | +dP/dtmax |

Cell transplantation was performed shortly
after MI and followed 6 weeks

| | | | |
|---|---|---|---|
| Control-1 | 69.9 ± 1.7 | 11.5 ± 0.6 | 1671 ± 79 |
| (n = 7) | | | |
| hMSCs + hFCs | 89.7 ± 2.8**^{#} | 11.6 ± 1.0 | 1895 ± 51** |
| (n = 6) | | | |
| hMSCs alone | 75.3 ± 3.2 | 10.4 ± 0.7 | 1717 ± 94 |
| (n = 7) | | | |
| hMSCs + hFCs | 77.5 ± 3.2* | 10.2 ± 1.2 | 1775 ± 74* |
| without cyclosporine | | | |
| (n = 6) | | | |

Cell transplantation was performed 2 weeks
after MI and followed 6 weeks

| | | | |
|---|---|---|---|
| Control-2 | 53.2 ± 3.1 | 8.8 ± 1.0 | 1158 ± 80 |
| (n = 5) | | | |
| hMSCs + hFSc | 66.3 ± 5.3* | 8.3 ± 0.6 | 1594 ± 104* |
| (n = 6) | | | |

Values are mean ± SE.
Control-1, post-MI pig hearts transplanted with medium performed shortly after MI;
hMSCs + hFCs, post-MI pig hearts co-transplanted with hMSCs plus hFCs and administered with cyclosporin A;
Control-2, post-MI pig hearts transplanted with medium performed 2 weeks after MI.
*P < 0.05 vs. Control-1;
**P < 0.01 vs. Control-1;
^{#}P < 0.05 vs. hMSCs alone and hMSCs + hFCs without cyclosporine A.

TABLE E6
Hemodynamics and left ventricular blood flow measurements
in postinfarcted pig hearts at 6 weeks after cell transplantation

| | MI Control | MI-hMSC | MI-hMSCs + hFCs |
|---|---|---|---|
| Resting state | | | |
| LVSP | 70 ± 2 | 75 ± 3^{a} | 89 ± 3^{a,b} |
| LVEDP | 11.5 ± 0.6 | 10.4 ± 0.7 | 9.1 ± 0.7^{a} |
| +dP/dtmax | 1671 ± 79 | 1717 ± 94 | 1893 ± 51^{a} |
| Pacing stress | | | |
| LVSP | 28 ± 4 | 36 ± 4 | 40 ± 7^{a} |
| LVEDP | 18 ± 3 | 15 ± 4 | 14 ± 3^{a} |
| +dP/dtmax | 674 ± 90 | 713 ± 64 | 778 ± 84^{a} |

Data are presented as mean ± SE. LVSP, left ventricular systolic pressure (mmHg); LVEDP, left ventricular end-diastolic pressure (mmHg); +dP/dtmax, the maximum arising rate of peak left ventricular systolic pressure (mmHg/s); Blood flow, measured by microsphere technique (ml/min/g). Rest, the values at basal condition; Pacing stress, the values at pacing stress (180 beats/min).
^{a}P < 0.05 vs. MI Control;
^{b}P < 0.05 vs. MI-hMSCs.

TABLE E7
Echocardiographic measurements of ventricular function in vivo 6
weeks after cell transplantation in porcine hearts.

| | MI Control | MI-hMSC | MI-hMSCs + hFCs |
|---|---|---|---|
| PW th (%) | 0.68 ± 0.05 | 0.72 ± 0.06 | 0.76 ± 0.08 |
| AW th (%) | 0.54 ± 0.07 | 0.70 ± 0.18 | 0.65 ± 0.09 |
| En FS (%) | 0.31 ± 0.03 | 0.38 ± 0.01^{a} | 0.42 ± 0.01^{a,b} |
| MW FS (%) | 0.17 ± 0.01 | 0.19 ± 0.01 | 0.24 ± 0.01^{a,b} |
| SV (ml/beat) | 45.7 ± 4.2 | 52.9 ± 5.0 | 73.1 ± 18.4 |
| CI (ml/min/gm) | 194.7 ± 16.0 | 234.8 ± 27.0 | 280.6 ± 28.9^{a} |

Values are mean ± SE. MI-Control, postinfarcted pigs with transplantation of the cell-free medium; MI-hMSCs, postinfarcted pigs with transplantation of hMSCs alone; MI-hMSCs + hFCs, postinfarcted pigs with co-transplantation of hMSCs plus hFCs. PW th, posterior relative wall thickness; AW th, anterior relative wall thickness; En FS, endocardial fractional shortening; MW FS, mid-wall fractional shortening; SV, stroke volume; CI, cardiac index.
^{a}P < 0.05 vs. MI Control;
^{b}P < 0.05 vs. MI-hMSCs.

FIG. 22 as a whole graphically provides hemodynamic measurements in postinfarcted porcine hearts before ligation (Baseline); at one hour; and at 6 weeks after MI. Cell transplantation of hMSCs alone, or of hMSCs plus hFC, improved the ventricular function compared to the MI control animals. There is a trend of greater beneficial effects on ventricular function with co-transplantation of hMSCs plus hFCs compared to transplantation of hMSCs alone. MI-Control, postinfarcted pigs with transplantation of the cell-free medium (n=7); MI-hMSCs, postinfarcted pigs with transplantation of hMSCs alone (n=6); MI-hMSCs+hFCs, postinfarcted pigs with co-transplantation of hMSCs plus hFCs (n=7). FIG. 22A shows LVSP, the left ventricular systolic pressure; FIG. 22B illustrates LVEDP, the left end-diastolic pressure; FIGS. 22C shows +dP/dt, the peak rate of pressure rise; and FIG. 22D shows −dP/dt, the peak rate of pressure fall. * P<0.05, ** P<0.01 vs. MI-Control at 6 weeks after MI; # P<0.05 vs. MI-hMSCs 6 weeks after MI.

### Experiment No. 14: Echocardiographic Parameters

As clinically measured, the baseline echocardiographic parameters were similar among the animals with or without cell transplantation (data not shown). Six weeks after MI operation, left ventricular contractility was decreased in MI pigs injected with the cell-free medium. This is revealed by the data presented by Table E8.

TABLE E8
Echocardiographic measurements of ventricular function in vivo 6
weeks after MI induction and cell transplantation in porcine hearts.

| | Control | hMSCs | hMSCs + hFCs |
|---|---|---|---|
| | (n = 7) | (n = 6) | (n = 7) |
| PW th (%) | 0.68 ± 0.05 | 0.72 ± 0.06 | 0.76 ± 0.08 |
| AW th (%) | 0.54 ± 0.07 | 0.70 ± 0.18 | 0.65 ± 0.09 |
| En FS (%) | 0.31 ± 0.03 | 0.38 ± 0.01* | 0.42 ± 0.01**# |
| MW FS (%) | 0.17 ± 0.01 | 0.19 ± 0.01 | 0.24 ± 0.01**# |
| SV (ml/beat) | 45.7 ± 4.2 | 52.9 ± 5.0 | 73.1 ± 18.4 |
| CI (ml/min/gm) | 194.7 ± 16.0 | 234.8 ± 27.0 | 280.6 ± 28.9* |
| Values are mean ± SE. Control, postinfarcted pigs with injection of the cell-free medium; hMSCs, postinfarcted pigs with transplantation of hMSCs alone; hMSCs + hFCs, postinfarcted pigs with co-transplantation of hMSCs plus hFCs. PW th, posterior relative wall thickness; AW th, anterior relative wall thickness; En FS, endocardial fractional shortening; MW FS, mid-wall fractional shortening; SV, stroke | | | |
| # volume; CI, cardiac index. | | | |

*P < 0.05; **P < 0.01 vs; Control; #P < 0.05; vs. hMSCs.

As shown by Table E8, cell transplantation significantly improved ventricular function reflected by an increase of endocardial fractional shortening, mid-wall fractional shortening, stroke volume and cardiac index. The beneficial effects were more profound in MI pigs that received co-transplantation of hMSCs plus hFCs than in animals transplanted with hMSCs alone.

### Experiment No. 15: Histological Assessments

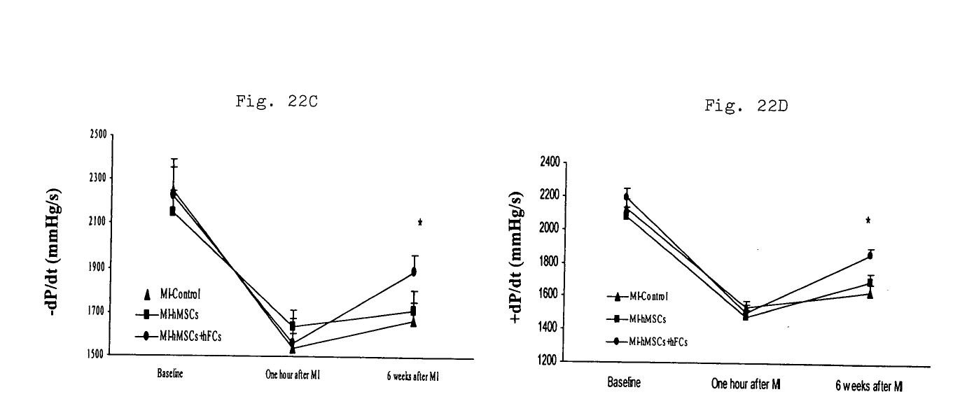

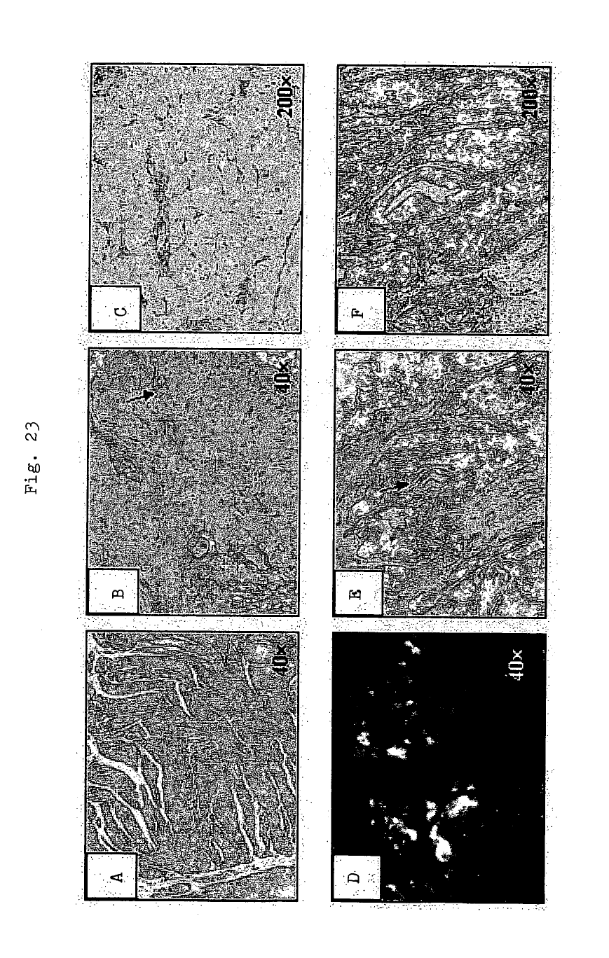

Histologic staining of myocardial sections with hematoxylin and eosin confirmed fibrous scar tissue in infarcted areas at 6 weeks after MI operation and cell transplantation. This is revealed by the stained sections illustrated by FIGS. 23A-23F respectively.

FIG. 23 as a whole illustrates the morphology of H & E staining of: normal porcine myocardium (FIG. 23A); and infarcted myocardium with medium injection (FIGS. 23B and 23C). In comparison, FIG. 23D shows GFP positive clusters sectioned from a MI pig heart with co-transplantation of hMSCs plus hFCs. The H & E staining of infarcted porcine myocardium with co-transplantation of hMSCs plus hFCs is shown by FIGS. 23E and 23F. The arrows in FIGS. 23B and 23E point to the areas corresponding with the magnification seen in FIGS. 23C and 23F.

In particular, it will be noted and appreciated that engrafted cells were diversely distributed in infarcted areas transplanted with hMSCs plus hFCs (FIGS. 23E and 23F) and hMSCs alone (data not shown). Fibrosis without regenerated cell islets was found in infarcted myocardium injected with the cell-free medium (FIGS. 23B and 23C). Positive GFP spots observed under fluorescent microscopy demonstrated the survival of engrafted cells in myocardium transplanted with hMSCs plus hFCs (FIG. 23D) or with hMSCs alone (data not shown).

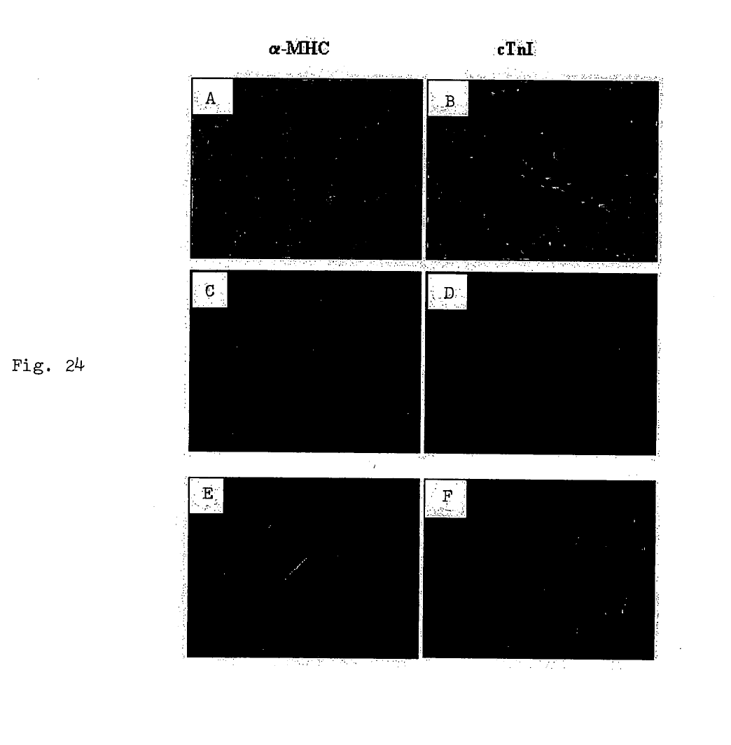

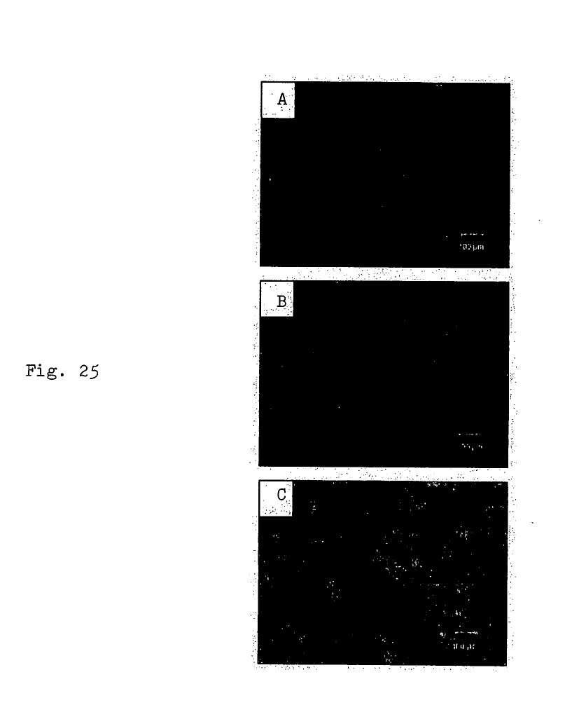

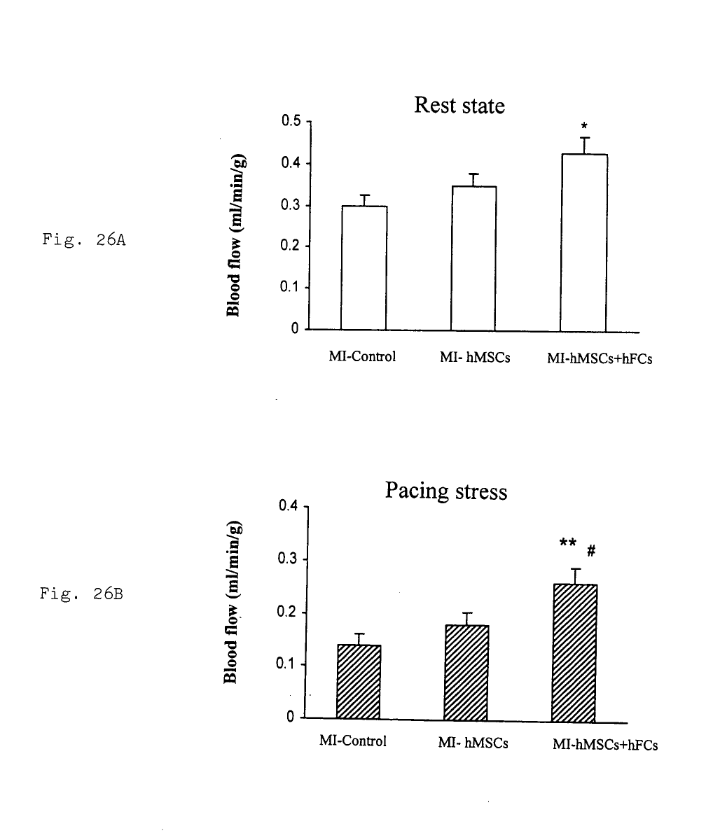

Furthermore, the intensity of immunostaining for a-cardiac myosin heavy chain (α-MHC, FIG. 24, left column) and cardiac troponin I (cTnI, FIG. 24, right column) was much higher in infarcted myocardium with co-transplantation of hMSCs plus hFCs than with injection of the cell-free medium. Double staining for GFP and cTnI of injured myocardium co-transplanted with hMSCs plus hFCs was shown in FIG. 25, which further confirms that implanted stem cells could differentiate into cardio-like cells.

FIGS. 24A-24F are photographic illustrations showing positive immunofluorescent stains to a-MHC and cTnI. These were found in normal myocardium (FIGS. 24A and 24B respectively), and in postinfarcted myocardium transplanted with hMSCs plus hFCs (FIGS. 24E and 24F respectively); but not in injured porcine myocardium with medium injection (FIGS. 24C and 24D respectively). The results were obtained from different animals for fluorescent labeling of αMHC and cTnI. Magnification: ×200.

FIGS. 25A-25C are photogenic illustrations showing double staining for GFP and cTnI of injured myocardium co-transplanted with hMSCs plus hFCs. FIGS. 25A and 25B show the staining of GFP by a monoclonal anti-GFP antibody and of cTnI by a polyclonal anti-cTnI antibody, respectively. FIG. 25C shows the merger of GFP and cTnI staining and demonstrates that engrafted GFP positive cells differentiated into cardiac myocytes. Magnification: ×200.

### Experiment No. 16: Improvements in Regional Blood Flow

In addition, to evaluate cardiogenesis in infarcted myocardium with cell transplantation, regional blood flow of the myocardium was measured by neutron microspheres. The results are graphically illustrated by FIGS. 26A-26B and by Table E9.

TABLE E9
Blood flow measurement with microsphere technique in post-MI
porcine hearts.

| | |
|---|---|
| Rest | Pacing stress |

Cell transplantation was performed shortly after MI and followed 6
weeks

| | | |
|---|---|---|
| Control (n = 6) | 0.26 ± 0.05 | 0.12 ± 0.04 |
| hMSCs + hFCs (n = 7) | 0.47 ± 0.08** | 0.23 ± 0.01** |
| hMSCs alone (n = 6) | 0.33 ± 0.06 | 0.21 ± 0.01* |
| hMSCs + hFCs (n = 5) | 0.41 ± 0.04** | 0.17 ± 0.05 |
| (no cyclosporine) | | |

Cell transplantation was performed 2 weeks after MI and followed 6
weeks

| | | |
|---|---|---|
| Control (n = 5) | 0.32 ± 0.05 | 0.13 ± 0.04 |
| hMSCs + hFCs (n = 3) | 0.38 ± 0.04 | 0.28 ± 0.04** |

Values are mean ± SE. Control-1, post-MI pig hearts transplanted with medium performed shortly after MI; hMSCs + hFCs, post-MI pig hearts co-transplanted with hMSCs plus hFCs and administered with cyclosporine A; Control-2, post-MI pig hearts transplanted with medium performed 2 weeks after MI.
*P < 0.05, **P < 0.01; vs. Control.

FIGS. 26A and 26B are graphs showing the recorded blood flow measurements with the neutron microsphere technique in postinfarcted porcine hearts at resting condition (FIG. 26A) and with pacing stress (FIG. 26B). MI-Control, postinfarcted pigs with transplantation of the cell-free medium (n=7); MI-hMSCs, postinfarcted pigs with transplanation of MI-hMSCs (n=6); MI-hMSCs+hFCs, postinfarcted pigs with co-transplantation of hMSCs plus hFCs (n=7). * P<0.05, ** P<0.01 vs. MI-Control; # P<0.05 vs. MI-hMSCs.

As the empirical data shows graphically, six weeks after MI induction, resting blood flow was significantly decreased in control MI myocardium injected with the cell-free medium. Compared to control MI hearts, cell transplantation increased blood flow in infarcted myocardium. The increase was even greater in pigs co-transplanted with hMSCs plus hFCs than in animals transplanted with hMSCs alone. In addition, the beneficial effects on blood flow were also observed under the pacing-induced stress condition in MI hearts with cell transplantation. Again, co-transplantation of hMSCs plus hFCs produced a greater increase in blood flow during pacing.

### Conclusions Based on Empirical Results

1. Human adult stem cells are alternative cell sources for clinical application. The data of this study demonstrates that intramyocardial transplantation of hMSCs improved infarcted heart function and the improvement was even greater in MI pigs co-transfected with hMSCs and hFCs.

2. In the present study, transplantation of hMSC alone improved cardiac function in infarcted pigs 6 weeks after MI induction and cell implantation, but co-transplantation of hMSCs plus hFCs produced much greater functional recovery. Engrafted hMSCs or hMSCs plus hFCs not only survived in injured porcine myocardium, but also generated new myocardium evidenced by positive staining for α-MHC and cTnI.

3. The present data suggest that the improvement of ventricular function, at least partially, results from new cardiomyocytes differentiated from engrafted hMSC cells. One explanation for the lesser improvement of cardiac function in MI hearts transplanted with hMSCs alone is that engrafted hMSCs may not yield sufficient myocyte numbers to repair the injured myocardium. In contrast, implanted hFCs survive in injured myocardium and form more new myocardium to further improve heart function in MI pigs with co-transplantation of hMSCs plus hFCs.

4. In the present study, the blood supply to the ischemic territory, as assessed by neutron microsphere method, was improved by the transplantation of hMSCs alone and hMSCs plus hFCs. Engrafted cells thus provide the cell source for formation of new blood vessels and release vascular endothelial growth factor to induce new capillary formation and growth in injured myocardium.

5. Transplantation of hMSCs alone or hMSCs plus hFCs can regenerate injured myocardium and improve cardiac function in our MI porcine model. However, the improvement is much greater in MI hearts co-transplanted with hMSCs plus hFCS. The better heart function in co-transplanted MI animals apparently results from the regeneration of more myocardium and a stronger effect on angiogenesis caused by engrafted hFCs.

The present invention is not to be limited in scope nor restricted in form except by the claims appended hereto.

What we claim is:

1. A therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising not less than two different types of cells in combination, said two different types being selected from the group consisting of at least one identifiable type of stem cells, at least one identifiable type of progenitor cells, at least one identifiable type of lineage-committed cells, at least one identifiable type of partially-differentiated cells, and at least one identifiable type of completely differentiated cells; and
allowing said introduced inoculum of cells to develop in-situ as integrated cells within and about the heart, whereby the severity of cardiac dysfunction becomes decreased in the afflicted living subject.

2. A therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising not less than two different types of cells in combination, said two different types being selected from the group consisting of one or more identifiable types of stem cells, one or more identifiable types of progenitor cells, one or more identifiable types of lineage-committed cells, and one or more identifiable types of partially-differentiated cells; and
allowing said introduced inoculum of cells to develop in-situ as integrated cells within and about the heart, whereby the severity of cardiac dysfunction becomes decreased in the afflicted living subject.

3. A therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising not less than three different types of cells in combination, said three different types being selected from the group consisting of one or more identifiable types of stem cells, one or more identifiable types of progenitor cells, one or more identifiable types of lineage-committed cells, one or more identifiable types of partially-differentiated cells, and one or more identifiable types of completely differentiated cells; and
allowing said introduced inoculum of cells to develop in-situ as integrated cells within and about the heart, whereby the severity of cardiac dysfunction becomes decreased in the afflicted living subject.

4. The therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising at least one identifiable type of stem cell and at least one identifiable type of progenitor cell in combination; and
allowing said introduced inoculum of cells to develop in-situ as integrated cells within and about the heart, whereby the severity of cardiac dysfunction becomes decreased in the afflicted living subject.

5. A therapeutic method for decreasing the severity of cardiac dysfunction in a living mammalian afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising at least one identifiable type of progenitor cell, and at least one identifiable type of lineage-committed cell in combination; and
allowing said introduced inoculum of cells to develop in-situ as integrated cells within and about the heart, whereby the severity of cardiac dysfunction becomes decreased in the afflicted living subject.

6. A therapeutic method for decreasing the severity of cardiac dysfunction in a living mammalian afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising at least one identifiable type of lineage-committed cell and at least one identifiable type of partially-differentiated cell in combination; and
allowing said introduced inoculum of cells to develop in-situ as integrated cells within and about the heart, whereby the severity of cardiac dysfunction becomes decreased in the afflicted living subject.

7. A therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising at least one identifiable type of stem cell and at least one identifiable type of partially-differentiated cell in combination; and
allowing said introduced inoculum of cells to commit to develop in-situ as integrated cells within and about the heart, whereby the severity of cardiac dysfunction becomes decreased in the afflicted living subject.

8. A therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising at least one identifiable type of progenitor cell and at least one identifiable type of partially-differentiated cell in combination; and
allowing said introduced inoculum of cells to develop in-situ as integrated cells within and about the heart, whereby the severity of cardiac dysfunction becomes decreased in the afflicted living subject.

9. The therapeutic method for decreasing the severity of cardiac dysfunction as recited in claim 2, 4, 5, 6, 7 or 8 wherein said prepared inoculum further comprises at least one type of completely differentiated cell.

10. The therapeutic method for decreasing the severity of cardiac dysfunction as recited in claim 1, 2, 3, 4, 5, 6, 7 or 8 wherein said identifiable types of cells comprising said mixture of said prepared inoculum are of the same derivation.

11. The therapeutic method for decreasing the severity of cardiac dysfunction as recited in claim 1, 2, 3, 4, 5, 6, 7 or 8 wherein said identifiable types of cells comprising said mixture of said prepared inoculum are of different derivations.

12. The therapeutic method for decreasing the severity of cardiac dysfunction as recited in claim 4 or 7 wherein said identifiable type of stem cell is selected from the group consisting of embryonic stem cells, mesenchymal stem cells, hematopoietic stem cells, neural stem cells and neural crest stem cells.

13. The therapeutic method for decreasing the severity of cardiac dysfunction as recited in claim 4 or 8 wherein said identifiable type of progenitor cell is selected from the group consisting of embryonic progenitor cells, mesenchymal progenitor cells, hematopoietic progenitor cells, neural progenitor cells and neural crest progenitor cells.

14. The therapeutic method for decreasing the severity of cardiac dysfunction as recited in claim 5 or 6 wherein said identifiable type of lineage-committed cell is selected from the group consisting of lineage-committed cells of embryonic stem cell origin, lineage-committed cells of mesenchymal stem cell origin, lineage-committed cells of hematopoietic stem cell origin, lineage-committed cells of neural stem cell origin and lineage-committed cells of neural crest stem cell origin.

15. The therapeutic method for decreasing the severity of cardiac dysfunction as recited in claim 6, 7 or 8 wherein said identifiable type of partially-differentiated cell is selected from the group consisting of partially-differentiated cells of embryonic stem cell origin, partially-differentiated cells of mesenchymal stem cell origin, partially differentiated cells of hematopoietic stem cell origin, partially differentiated cells of neural stem cell origin and partially-differentiated cells of neural crest stem cell origin

16. A therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising not less than two identiable types of stem cells in combination; and
allowing said introduced inoculum of cells to develop in-situ as integrated cells within and about the heart, whereby the severity of cardiac dysfunction becomes decreased in the afflicted living subject.

17. A therapeutic method for decreasing the severity of cardiac dysfunction in a living mammalian afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising not less than two identiable types of progenitor cells in combination; and
allowing said introduced inoculum of cells to develop in-situ as integrated cells within and about the heart, whereby the severity of cardiac dysfunction becomes decreased in the afflicted living subject.

18. A therapeutic method for decreasing the severity of cardiac dysfunction in a living mammalian afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising not less than two identiable types of lineage-committed cells of differing derivations in combination;
allowing said introduced inoculum of cells to develop in-situ as integrated cells within and about the heart, whereby the severity of cardiac dysfunction becomes decreased in the afflicted living subject.

19. A therapeutic method for decreasing the severity of cardiac dysfunction in a living mammalian subject afflicted with a clinically recognized form of cardiopathology, said method comprising the steps of:
introducing a prepared inoculum of viable mammalian cells to at least a portion of the heart in the afflicted living subject, said prepared inoculum being a mixture of mammalian cells comprising not less than two identifiable types of partially-differentiated cells of differing derivations in combination; and
allowing said introduced inoculum of cells to develop in-situ as integrated cells within and about the heart, whereby the severity of cardiac dysfunction becomes decreased in the afflicted living subject.

20. The therapeutic method for decreasing the severity of cardiac dysfunction as recited in claim 16, 17, 18 or 19 wherein said prepared inoculum further comprises at least one identifiable type of differentiated cell.

21. The therapeutic method for decreasing the severity of cardiac dysfunction as recited in claim 1, 2, 3, 4, 5, 6, 7, 8, 16, 17, 18 or 19 wherein the recognized form of cardiopathology for the afflicted living subject is a myocardial infarction.

22. The therapeutic method for decreasing the severity of cardiac dysfunction as recited in claim 1, 2, 3, 4, 5, 6, 7, 8, 16, 17, 18 or 19 wherein the recognized form of cardiopathology for the afflicted living subject is heart failure.

23. The therapeutic method for decreasing the severity of cardiac dysfunction as recited in claim 1, 2, 3, 4, 5, 6, 7, 8, 16, 17, 18 or 19 wherein the recognized form of cardiopathology for the afflicted living subject is myocarditis.

24. The therapeutic method for decreasing the severity of cardiac dysfunction as recited in claim 1, 2, 3, 4, 5, 6, 7, 8, 16, 17, 18 or 19 wherein the recognized form of cardiopathology for the afflicted living subject is cardiac dysrhythmia.

25. The therapeutic method for decreasing the severity of cardiac dysfunction as recited in claim 1, 2, 5, 6, 7, 8, 16, 17, 18 or 19 wherein said cells comprising said prepared inoculum are derived from an organ of a mammal.

26. The therapeutic method for decreasing the severity of cardiac dysfunction as recited in claim 1, 2, 5, 6, 7, 8, 16, 17, 18 or 19 wherein said cells comprising said prepared inoculum are derived from an organ of a human donor.

27. The therapeutic method for decreasing the severity of cardiac dysfunction as recited in claim 25 or 26 wherein said organ is selected from the group consisting of bone marrow, peripheral blood, placenta, or umbilical cord blood.
